(19) United States
(12) Patent Application Publication
(10) Pub. No.: US 2024/0425501 A1
(43) Pub. Date: Dec. 26, 2024

(54) NOVEL BICYCLIC HETEROARYL DERIVATIVES AS SOS1:KRAS PROTEINPROTEIN INTERACTION INHIBITORS

(71) Applicants: Sanjita SASMAL, Hyderabad (IN); Venkatesham BOORGU, Hyderabad (IN); Ashok ETTAM, Hyderabad (IN); SATYARX PHARMA INNOVATIONS PVT LTD, Hyderabad (IN)

(72) Inventors: Sanjita SASMAL, Hyderabad (IN); Venkatesham BOORGU, Hyderabad (IN); Ashok ETTAM, Hyderabad (IN)

(21) Appl. No.: 18/702,429

(22) PCT Filed: Oct. 20, 2022
(86) PCT No.: PCT/IB2022/060096

§ 371 Date: Apr. 18, 2024

(30) Foreign Application Priority Data
Oct. 21, 2021 (IN) 202141047843

(51) Int. Cl. C07D 471/04; A61K 31/5025; A61K 31/517; A61K 31/519; A61K 31/5377; C07D 403/04; C07D 413/04

(52) U.S. Cl. CPC C07D 471/04; A61K 31/5025; A61K 31/517; A61K 31/519; A61K 31/5377; C07D 403/04; C07D 413/04

(57) ABSTRACT

The present invention relates to novel compounds of formula (I) which are inhibitors of SOS1:KRAS interaction, their pharmaceutically acceptable esters, salts, solvates, isomers thereof. The present invention specifically relates to novel compounds of formula (I)

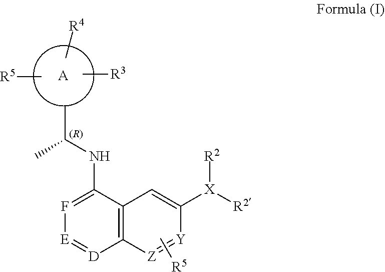

or a pharmaceutically acceptable salt or a pharmaceutically acceptable regioisomer thereof and processes for their preparation.

### FIELD OF INVENTION

The present invention relates to novel compounds of formula (I), their pharmaceutically acceptable salts, solvates, polymorphs, tautomers, optical and geometric isomers thereof, which are inhibitors of SOS1:KRAS protein-protein interaction.

The present invention also relates to a process for their manufacture derivatives of formula (I), pharmaceutical compositions containing them, and their use for the treatment or use in the treatment of cancer.

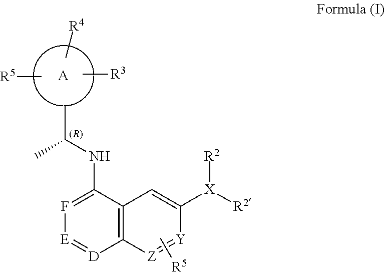

### BACKGROUND OF THE INVENTION

Son of Sevenless 1 (SOS1) is a human homologue of the originally identified Drosophila protein Son of Sevenless (Pierre et al., Biochem. Pharmacol., 201 1, 82(9): 1049-56; Chardin et al., Cytogenet. Cell. Genet., 1994, 66(1):68-9). The SOS1 protein consists of 1333 amino acids (150 kDa). SOS1 is a multi-domain protein with two tandem N-terminal histone domains (HD) followed by the Dbl homology domain (DH), a Pleckstrin homology domain (PH), a helical linker (HL), RAS exchanger motif (REM), CDC25 homology domain and a C-terminal proline rich domain (PR). In the native state, the GEF activity of SOS family of proteins is autoinhibited by its own N terminal autoinhibitory domain, and is activated only upon membrane recruitment (Science, 2019, 363, 1098-1103)

SOS1 and SOS2 are the most widely expressed guanidine exchange factors (GEFs) that are responsible for activation of RAS and RAC proteins (BBA Reviews on Cancer, Volume 1874, Issue 2, December 2020, 188445). SOS1 and SOS2 appear to be ubiquitously expressed, as the presence of specific RNAs or proteins for those genes is detectable in practically all human cells, organs and tissues tested. Constitutive KO SOS1 gene in mouse showed that SOS1 is essential for intrauterine and placental development, with constitutive-null-animals dying during mid-gestation due to defects of the embryos (EMBO J 19, 2000, 642-654; Genes Dev, 1997, 11, 309-320). In contrast, adult SOS2-constitutive-KO mice were perfectly viable and fertile and did not show any obvious phenotypic abnormalities [Mol. Cell. Biol, 20, 2000, 6410-6413]. SOS1 is the most crucial player in this nucleotide exchange reaction It promotes the exchange of Ras-bound GDP by GTP (PubMed:8493579). The mechanism is probably by promoting Ras activation, regulates phosphorylation of MAP kinase MAPK3 in response to EGF (PubMed:17339331). Catalytic component of a trimeric complex that participates in transduction of signals from Ras to Rac by promoting the Rac-specific guanine nucleotide exchange factor (GEF) activity. SOS-mediated activation of WT RAS is critical for the development of RAS-mutant tumors (Nat. Communications 2012, 3, 1168; Enzym, 2013, 25-39; Cancer Discovery 2013, 3, 113-123)

The initial proof of concept for evaluating SOS1 as a pharmacological target to treat cancer was obtained based on some genetic ablation studies. SOS1 KO/siRNA in mouse resulted in reduction in leukemogenesis (Leukemia, 2018, 32, 820-827; Blood, 2018, 132, 2575-2579), skin cancers (Mol. Cell. Biol. 2018, 38, e00048-18) and gastroesophageal cancers (Nat Med 2018, 24, 968-977). SOS1 is the second most common mutation in Noonan Syndrome (˜16.5%), an autosomal dominant disorder and the most predominant RASopathic condition (Mol Syndromol, 2010, 1, 2-26; Eur. J. of Med. Genetics, 2010 53, 322-324).

SOS1 is significantly mutated in lung adenocarcinoma (LUAD) patients who do not present canonical gain of function mutations in receptor tyrosine kinase pathway players like RAS/RAF, etc. (Genes, chromosomes & cancer. 2008; 47(3):253-9; Mol Cancer Res. 2019 April; 17(4): 1002-1012). Somatic SOS1 mutations are reported in rare and sporadic cancers like lung squamous cell carcinoma (Nat. Genetics, 2016, 48, 607-616), uterine corpus endometrial carcinoma (Cell. 2018 April 05; 173(2): 321-337), urothelial bladder cancer, liver hepatocellular carcinoma, acute myeloid leukemia (Int J Hematol, 88, 460-462, lower grade glioma and cutaneous melanoma (Ophthalmic Genet. 40 (2019) 22-28. ; Genes Chromosoma and Cancer 2008, 47, 253-259). SOS1 mutations are also reported in melanoma patients devoid of NRAS or BRAF mutations (Plos Genetics 2016, 12, e1006081). In vitro studies demonstrated SOS1 overexpression in cell lines derived from renal, bladder or prostate carcinomas (Oncogene, 1996, 12, 1097, 1107; IUBMB Life 2000, 49, 317-320; J. Urol, 1997, 158, 908-911; Int J of Oncol, 2009, 35, 751-760). In contrast, SOS1 deficiency co-relates with loss of metastatic capacity in ovarian cancer (Cancer Res, 2010, 70, 9979-9990). Thus SOS1 can be considered as a promising therapeutic target for anticancer treatment.

Recently, several patent applications are published which disclose/claim different heterocyclic compounds some of which are given below:

WO 1995/019774 A1 of Warner-Lambert Company discloses bicyclic compounds capable of inhibiting tyrosine kinases of the epidermal growth factor receptor family having the following formula:

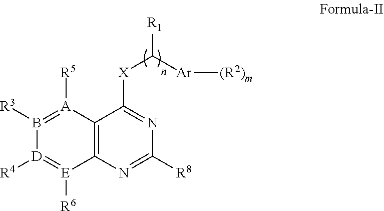

wherein all the groups have the meanings given therein, R³ is cycloalkyl (3-8 carbon atoms), R⁸ is alkyl of from 1-4 carbon atoms or amino or mono or dilower alkyl (1-4 carbon atoms) amino. However, this specification neither teaches nor enables specific compounds of the present invention.

WO 2020/020939 A1 of Faes Farma, S.A disclose compound of formula (I′)×

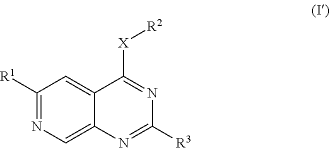

wherein all the groups are as defined therein. Even, the disclosure of this application does not specifically teach nor enable compounds of the present invention.

Treatment of cancer continues to be a challenge though several drugs that have been approved with different mechanism. In view of this there is unmet need for new drugs that can treat such diseases more effectively. We herein describe novel compounds which are inhibitors of SOS1:KRAS protein-protein interaction, which bind to the SOS1 catalytic site and simultaneously prevent interactions with and activation of RAS-family proteins. The novel compounds of formula (I) presented herein have inhibitory effect on the interaction of SOS1 and KRAS protein.

Accordingly, it is an object of the present invention to provide compounds of Formula (I) their pharmaceutically acceptable salts, solvates, polymorphs, tautomers, optical and geometric isomers thereof, as inhibitors of SOS1:KRAS protein-protein interaction.

### SUMMARY OF THE INVENTION

The present invention relates to novel compounds of formula (I) which are inhibitors of SOS1:KRAS protein-protein interaction, their pharmaceutically acceptable esters, salts, solvates, isomers thereof.

In one aspect of the present invention relates to novel compounds of formula (I)

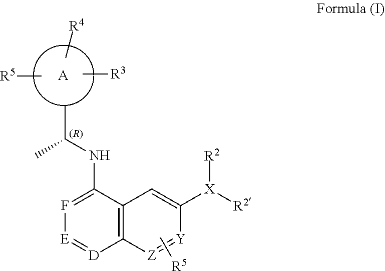

or a pharmaceutically acceptable salt or a pharmaceutically acceptable regioisomer thereof; wherein,
A is selected from 6-10 membered aryl, 5-10 membered heteroaryl and 3-10 membered heterocyclyl;
D, E, F is either CR¹ or N, with the option that either one or two of D, E, F are N and the rest being CR¹;
R¹ is selected from the group consisting of hydrogen, halogen, alkyl, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituents, with the exclusion that R1 is not alkyl when E is CR¹;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituents;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituents;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituents;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituents;
R^{1d} is selected from the group consisting of hydrogen, halogen, nitro, cyano, hydroxy, hydroxyl alkyl, amino, alkyl, alkoxy, alkenyl, alkynyl, wherein any of the group is optionally substituted by one or more, identical or different substituents;
R^{1e} is selected from the group consisting of hydrogen, halogen, nitro, cyano, hydroxy, hydroxyl alkyl, amino, alkyl, alkoxy, alkenyl, alkynyl, keto wherein any of the group is optionally substituted by one or more, identical or different substituents;
X is N or C(R^{1a}) or OR^{1c}, wherein R^{1a} and R^{1c} are as defined above;
Y is N or C(R^{1d}), wherein R^{1d} is as defined above;
Z is N or —C(O)— or C(R^{1e}), wherein R^{1e} is as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the atom to which they are attached form an optionally substituted 3-6 membered cycloalkyl, an optionally substituted 4-6 membered heterocyclyl containing 0-2 additional heteroatoms independently selected from O and N which is optionally substituted by one or more identical or different substituents;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituents;
R⁴ and R⁵ are same or different and each individually is selected from the group consisting of hydrogen, halogen, amino, nitro, alkyl or taken together when present on adjacent carbon atoms form an optionally substituted 4-6 membered aryl ring system containing optionally 1-2 heteroatoms independently selected from O and N.

In another aspect of the present invention, it relates to process for the preparation of novel compounds of formula (I).

In yet another aspect of the present invention, it relates to pharmaceutical composition comprising novel compounds of the formula (I) and processes for preparing thereof.

In yet further another aspect of the present invention, the invention relates to use of compounds of formula (I) and pharmaceutically acceptable derivatives, salts and regioisomers thereof, including mixtures thereof in all ratios as a medicament, by inhibiting SOS1 in treating diseases such as cancer.

### DETAILED DESCRIPTION OF THE INVENTION

According to another embodiment, specifically provided are compounds of formula (I), in which the group
is specifically represented by

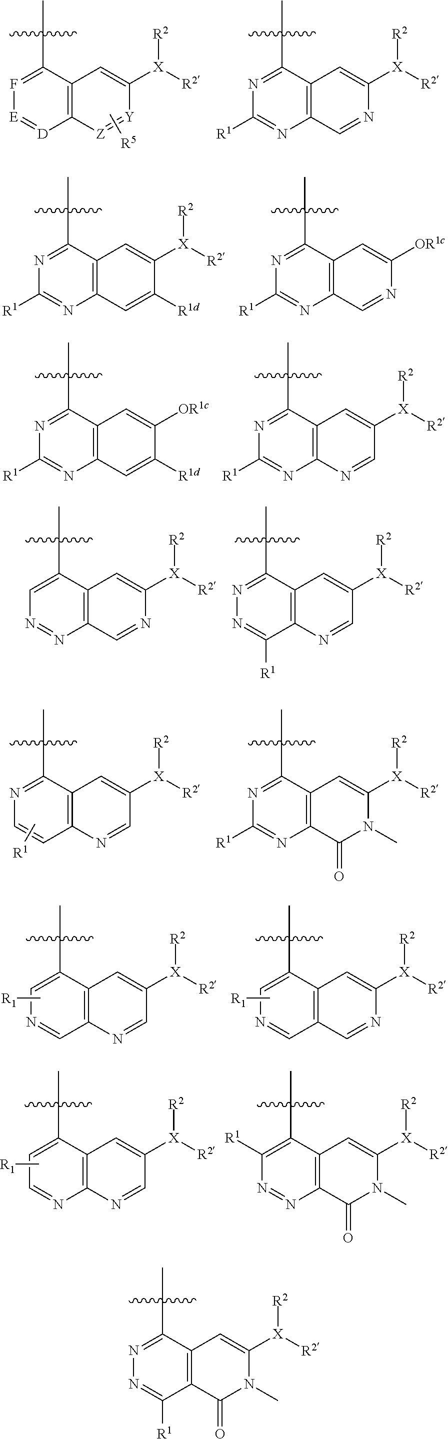

In another aspect the present invention relates to novel compounds of formula (Ia)

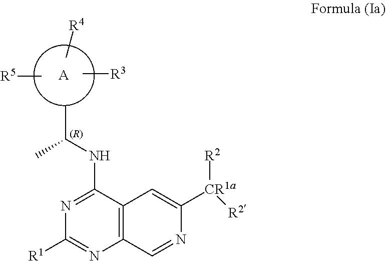

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆ₐlkyl), ═N—OH, alkyl, alkoxy, alkenyl, alkynyl, aryl, cycloalkyl, heteroaryl, heterocyclyl.

In yet another aspect the present invention relates to novel compounds of formula (Ib)

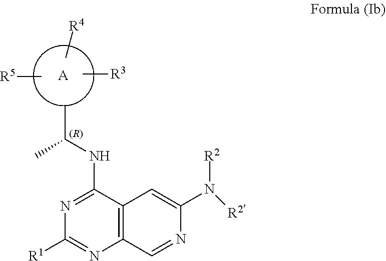

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} when present on nitrogen atom taken together with the nitrogen atom to which they are attached form an optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Ic)

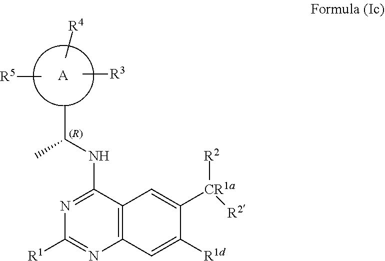

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1d} is selected from the group consisting of hydrogen, halogen, nitro, cyano, hydroxy, hydroxyl alkyl, amino, alkyl, alkoxy, alkenyl, alkynyl, wherein any of the group is optionally substituted by one or more, identical or different substituents selected from R⁶;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆ₐlkyl), ═N—OH, alkyl, alkoxy, alkenyl, alkynyl, aryl, cycloalkyl, heteroaryl, heterocyclyl.

In another aspect the present invention relates to novel compounds of formula (Id)

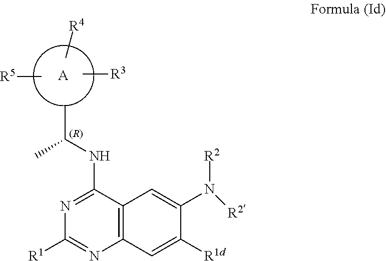

or a pharmaceutically acceptable salt or a pharmaceutically acceptable regioisomer thereof; wherein,
A is selected from 6 membered aryl, 4-6 membered heteroaryl and 4-6 membered heterocyclyl; R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1d} is selected from the group consisting of hydrogen, halogen, nitro, cyano, hydroxy, hydroxyl alkyl, amino, alkyl, alkoxy, alkenyl, alkynyl, wherein any of the group is optionally substituted by one or more, identical or different substituents selected from R⁶;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} when present on nitrogen atom taken together with the nitrogen atom to which they are attached form an optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Ie)

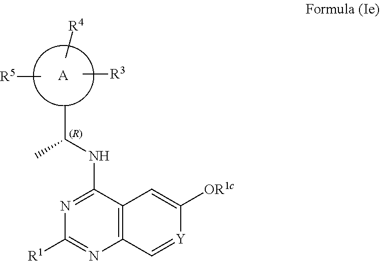

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituents selected from R⁶;
R^{1d} is selected from the group consisting of hydrogen, halogen, nitro, cyano, hydroxy, hydroxyl alkyl, amino, alkyl, alkoxy, alkenyl, alkynyl, wherein any of the group is optionally substituted by one or more, identical or different substituents selected from R⁶;
Y is N or C(R^{1d}), wherein R^{1d} is as defined above;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (If)

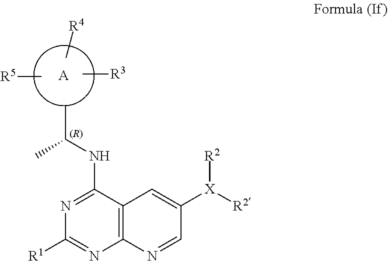

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶;
X is N or C(R^{1a}) or OR^{1c}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶; R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Ig)

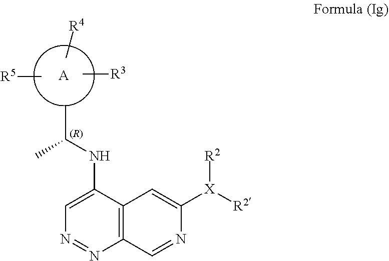

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶;
X is N or C(R^{1a}) or OR^{1c}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆ₐlkyl), ═N—OH, alkyl, alkoxy, alkenyl, alkynyl, aryl, cycloalkyl, heteroaryl, heterocyclyl.

In another aspect the present invention relates to novel compounds of formula (Ih)

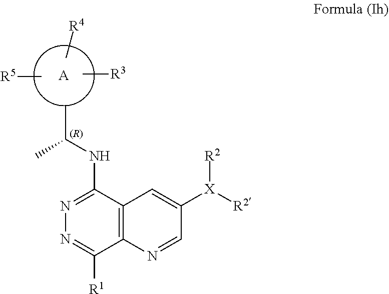

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶.

X is N or C(R^{1a}) or OR¹c, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Ii)

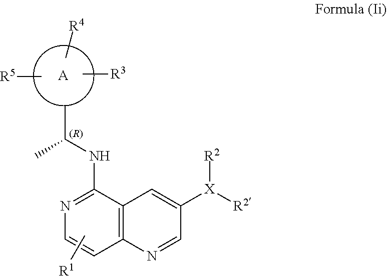

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶.

X is N or C(R^{1a}) or OR^{1C}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Ij)

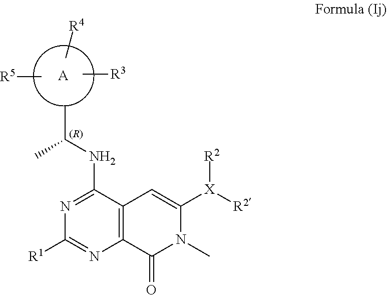

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶.

X is N or C(R^{1a}) or OR^{1c}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Ik)

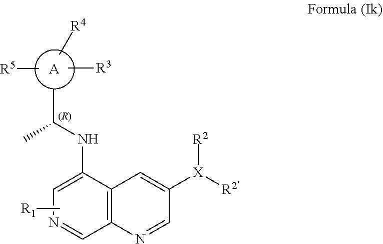

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶; or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1C} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶.

X is N or C(R^{1a}) or OR^{1c}, wherein R^{1a} and R^{1c} are as defined above; R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶; R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶; R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N; R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Il)

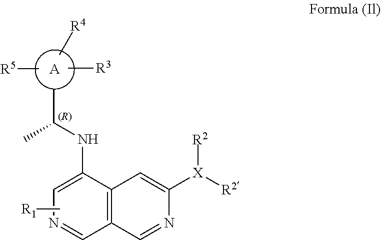

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶.
X is N or C(R^{1a}) or OR^{1c}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆ₐlkyl), ═N—OH, alkyl, alkoxy, alkenyl, alkynyl, aryl, cycloalkyl, heteroaryl, heterocyclyl.

In another aspect the present invention relates to novel compounds of formula (Im)

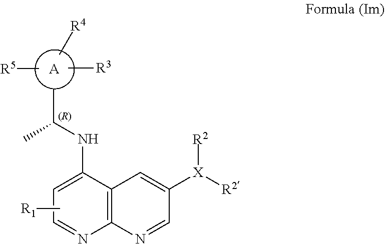

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶; R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶;
X is N or C(R^{1a}) or OR^{1c}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N; R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (In)

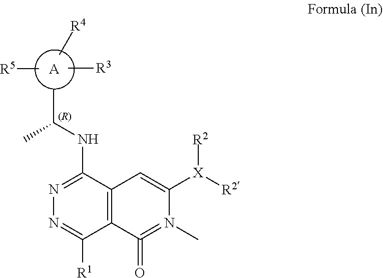

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶.
X is N or C(R^{1a}) or OR^{1c}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

In another aspect the present invention relates to novel compounds of formula (Jo)

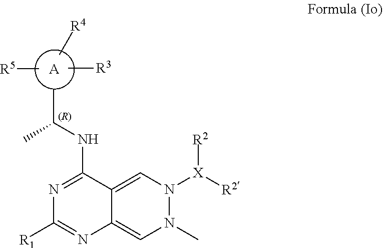

or a pharmaceutically acceptable salt or a pharmaceutically acceptable regioisomer thereof; wherein,
A is selected from 6 membered aryl, 4-6 membered heteroaryl and 4-6 membered heterocyclyl;
R¹ is selected from the group consisting of hydrogen, halogen, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituent selected from R⁶;
R^{1a} is selected from the group consisting of hydrogen, alkyl, —C(O)alkyl, —S(O)ₙalkyl, —S(O)ₙNH₂, alkenyl, alkynyl, cycloalkyl, heterocyclyl, aryl and heteroaryl, wherein any of the group is optionally substituted by one or more, identical or different substituent selected from R⁶;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituent selected from R⁶;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituent selected from R⁶;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituent selected from R⁶;
X is N or C(R^{1a}) or OR^{1c}, wherein R^{1a} and R^{1c} are as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a}, NR^{1a}R^{1b} wherein any of the group is optionally substituted by one or more, identical or different substituents; or R² and R^{2′} taken together with the carbon atom to which they are attached form optionally substituted 3-6 membered cycloalkyl, optionally substituted 4-6 membered heterocyclyl containing 0-2 additional heteroatoms independently selected from O and N which is optionally substituted by one or more identical or different substituent selected from R⁶;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituent selected from R⁶;
R⁴ and R⁵ are same or different and each individually is selected from the group consisting of hydrogen, halogen, amino, nitro, alkyl, or taken together when present on adjacent carbon atoms form an optionally substituted 4-6 membered aryl ring system containing optionally 1-2 heteroatoms independently selected from O and N;
R⁶ is selected from the group consisting of hydrogen, halogen, nitro, cyano, hydroxy, hydroxyl alkyl, amino, alkylamino, dialkylamino, oxo (═O), thiooxo (═S), carboxy, —C(O)O(C₁-C₆alkyl), —C(O)(C₁-C₆alkyl), —NH(C₁-C₆alkyl), ═N—OH, alkyl, alkoxy, alkenyl, alkynyl, aryl, cycloalkyl, heteroaryl, heterocyclyl.

According to another embodiment, specifically provided are compounds of formula (I), in which R³ is hydrogen; R⁴ and R⁵ are hydrogen, halogen, amino or alkyl.

According to another embodiment, specifically provided are compounds of formula (I), in which the group

is specifically represented by

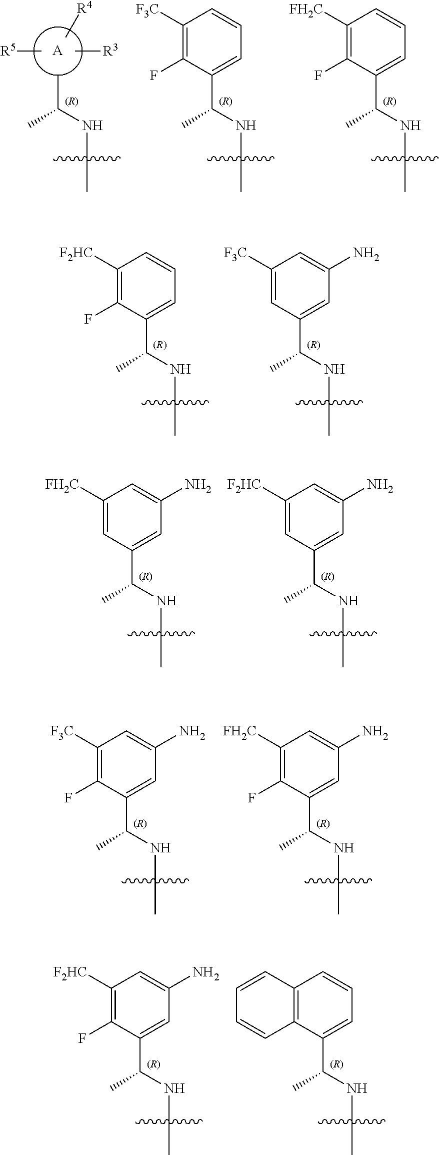

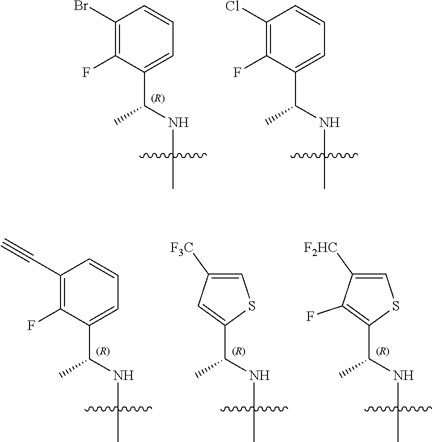

Without limiting the scope of present invention, the following definitions are provided in order to understand the detailed description of the present invention.

The term “Alkyl” as used herein refers and is not limited to a hydrocarbon chain that may be a linear or branched chain, containing the indicated number of carbon atoms, for example, a C₁-C₁₂ alkyl group may have from 1 to 12 (inclusive) carbon atoms in it. Examples of C₁-C₁₂ alkyl groups include, but are not limited to, methyl, ethyl, propyl, isopropyl, butyl, isobutyl, sec-butyl, tert-butyl, pentyl, isopentyl, neopentyl, hexyl, and isohexyl. An alkyl group can be unsubstituted or substituted with one or more suitable groups.

The term alkenyl as used herein refers and is not limited to a linear, branched unsaturated C₁-C₆ hydrocarbyl group containing a double bond, but are not limited to ethenyl, propenyl, butenyl. An alkenyl group can be unsubstituted or substituted with one or more suitable groups.

The term alkynyl as used herein refers and is not limited to a linear, branched unsaturated C₁-C₆ hydrocarbyl group containing a triple bond, but are not limited to acetylenyl, propynyl, butynyl. An alkynyl group can be unsubstituted or substituted with one or more suitable groups.

The term “Amino” as used herein refers and is not limited to an —N— group, the nitrogen atom of said group being attached to a hydrogen, alkyl, cycloalkyl, aryl, heterocyclyl or any suitable groups. An amino group can be unsubstituted or substituted with one or more of the suitable groups.

The term “Aryl” as used herein refers and is not limited to an optionally substituted monocylic, bicyclic or polycyclic aromatic carbocyclic ring system of about 6 to 14 carbon atoms. Examples of a C₆-C₁₄ aryl group include, but are not limited to phenyl, naphthyl, biphenyl, anthryl, tetrahydronaphthyl, fluorenyl, indanyl, biphenylenyl, and acenaphthyl. Aryl group which can be unsubstituted or substituted with one or more suitable groups.

The term “Halogen” or “halo” includes fluorine, chlorine, bromine or iodine.

“Hydroxy” refers to —OH group.

The term “cycloalkyl” as used herein refers and is not limited to a non-aromatic, saturated or partially saturated, monocyclic or polycyclic 3 to 10 member ring system. Exemplary cycloalkyl groups include cyclopropyl, cyclobutyl, cyclopentyl, cyclohexyl, and the like. A cycloalkyl group can be unsubstituted or substituted with one or more suitable groups.

The term “Heterocyclyl” as used herein refers and is not limited to a non-aromatic, saturated or partially saturated, monocyclic or polycyclic ring system of 3 to 10 member having at least one heteroatom or heterogroup selected from O, N, S, S(O), S(O)₂, NH and C(O). Exemplary heterocycloalkyl groups include tetrahydrofuranyl, tetrahydropyranyl, pyrrolidinyl, piperdinyl, piperazinyl, morpholinyl, thiomorpholinyl, 1,3-dioxolanyl, 1,4-dioxanyl, azetadine, oxetane, thietane and the like. A heterocycloalkyl group can be unsubstituted or substituted with one or more suitable groups.

The term “Heteroaryl” as used herein refers and is not limited to an unsaturated, monocyclic, bicyclic, or polycyclic aromatic ring system containing at least one heteroatom selected from oxygen, sulphur and nitrogen. Examples of C₅-C₁₀ heteroaryl groups include furan, thiophene, indole, azaindole, oxazole, thiazole, thiadiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic heteroaryl groups include those where a phenyl, pyridine, pyrimidine or pyridazine ring is fused to a 5 or 6-membered monocyclic heterocyclyl ring having one or two nitrogen atoms in the ring, one nitrogen atom together with either one oxygen or one sulfur atom in the ring, or one O or S ring atom. A heteroaryl group can be unsubstituted or substituted with one or more suitable groups.

“Optionally substituted or substituted” as used herein means that at least one hydrogen atom of the optionally substituted group has been substituted with suitable substitutions as exemplified but not limited to halogen, nitro, cyano, hydroxy, hydroxyl alkyl, alkoxy, amino, oxo (═O), thiooxo (═S), NH(C₁-C₆alkyl), —N(C₁-C₃alkyl)(C₁—C₆alkyl), —N(C₁-C₃alkyl)C(O)(C₁-C₆alkyl), —NHC(O)(C₁-C₆alkyl), —NHC(O)(cycloalkyl), —NHC(O)(aryl), —NHC(O)(heterocyclyl), —NHC(O)(heteroaryl), —NHC(O)H, —C(O)NH₂, —C(O)NH(C₁-C₆alkyl), —C(O)NH(cycloalkyl), —C(O)NH(heterocyclyl), —C(O)NH(heteroaryl), —C(O)N(C₁-C₆alkyl)(C₁-C₆alkyl), —S(O)NH(C₁-C₆alkyl), —S(O)₂NH(C₁-C₆alkyl), —S(O)NH(cycloalkyl), —S(O)₂NH(cycloalkyl), carboxy, —C(O)O(C₁—C₆alkyl), —C(O)(C₁-C₆alkyl), ═N—OH, substituted or unsubstituted alkyl, substituted or unsubstituted haloalkyl, substituted or unsubstituted alkoxy, substituted or unsubstituted haloalkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenylalkyl, substituted or unsubstituted cycloalkenyl, substituted or unsubstituted amino, substituted or unsubstituted heteroaryl, substituted or unsubstituted heterocyclyl.

The particular compounds of the present invention without departing from the scope of the definitions given under compounds of formula (I) and particular compounds emanated from formula (I) are summarized herein below encompassing the entirety of the scope of compounds within compound of formula (I).
1. (R)—N-(1-(3-amino-5-(trifluoromethyl)phenyl)ethyl)-2-chloro-6-(pyrrolidin-1-yl)pyrido[3,4-d]pyrimidin-4-amine
2. N4—(R)-(1-(3-amino-5-(trifluoromethyl)phenyl)ethyl)-N2,N2-dimethyl-6-(pyrrolidin-1-yl)pyrido[3,4-d]pyrimidine-2,4-diamine
3. (R)—N-(1-(3-amino-5-(trifluoromethyl)phenyl)ethyl)-2-methoxy-6-(pyrrolidin-1-yl)pyrido[3,4-d]pyrimidin-4-amine
4. (R)—N-(1-(3-amino-5-(trifluoromethyl)phenyl)ethyl)-2-(2-methoxyethoxy)-6-(pyrrolidin-1-yl)pyrido[3,4-d]pyrimidin-4-amine
5. (R)—N-(1-(3-amino-5-(trifluoromethyl)phenyl)ethyl)-2-morpholino-6-(pyrrolidin-1-yl)pyrido[3,4-d]pyrimidin-4-amine
6. (R)—N-(1-(3-amino-5-(trifluoromethyl)phenyl)ethyl)-2-(4-methylpiperazin-1-yl)-6-(pyrrolidin-1-yl)pyrido[3,4-d]pyrimidin-4-amine
7. (R)—N4-(1-(3-amino-5-(trifluoromethyl)phenyl)ethyl)-N2-methyl-6-(pyrrolidin-1-yl)pyrido[3,4-d]pyrimidine-2,4-diamine
8. (R)—N4-(1-(3-amino-5-(trifluoromethyl)phenyl)ethyl)-N2,N2-dimethyl-6-morpholinopyrido[3,4-d]pyrimidine-2,4-diamine
9. (R)—N4-(1-(3-amino-5-(trifluoromethyl)phenyl)ethyl)-N2-(2-(dimethylamino)ethyl)-6-(pyrrolidin-1-yl)pyrido[3,4-d]pyrimidine-2,4-diamine
10. (R)—N4-(1-(3-amino-5-(trifluoromethyl)phenyl)ethyl)-N2-(2-(dimethylamino)ethyl)-N2-methyl-6-(pyrrolidin-1-yl)pyrido[3,4-d]pyrimidine-2,4-diamine
11. (R)-2-((4-((1-(3-amino-5-(trifluoromethyl)phenyl)ethyl)amino)-6-(pyrrolidin-1-yl)pyrido[3,4-d]pyrimidin-2-yl)oxy)ethan-1-ol
12. (R)-2-((4-((1-(3-amino-5-(trifluoromethyl)phenyl)ethyl)amino)-6-(pyrrolidin-1-yl)pyrido[3,4-d]pyrimidin-2-yl)amino)ethan-1-ol
13. (R)-2-((4-((1-(3-amino-5-(trifluoromethyl)phenyl)ethyl)amino)-6-(pyrrolidin-1-yl)pyrido[3,4-d]pyrimidin-2-yl)(methyl)amino)ethan-1-ol
14. (R)—N4-(1-(3-amino-5-(trifluoromethyl)phenyl)ethyl)-N2-(2-methoxyethyl)-6-(pyrrolidin-1-yl)pyrido[3,4-d]pyrimidine-2,4-diamine
15. (R)—N4-(1-(3-amino-5-(trifluoromethyl)phenyl)ethyl)-N2-(2-methoxyethyl)-N2-methyl-6-(pyrrolidin-1-yl)pyrido[3,4-d]pyrimidine-2,4-diamine
16. (R)—N-(1-(3-amino-5-(trifluoromethyl)phenyl)ethyl)-2-(azetidin-1-yl)-6-(pyrrolidin-1-yl)pyrido[3,4-d]pyrimidin-4-amine
17. (R)—N-(1-(3-amino-5-(trifluoromethyl)phenyl)ethyl)-2,6-di(pyrrolidin-1-yl)pyrido[3,4-d]pyrimidin-4-amine
18. (R)—N4-(1-(3-amino-5-(trifluoromethyl)phenyl)ethyl)-N2-methyl-6-morpholinopyrido[3,4-d]pyrimidine-2,4-diamine
19. (R)—N-(1-(3-amino-5-(trifluoromethyl)phenyl)ethyl)-2-chloro-6-morpholinopyrido[3,4-d]pyrimidin-4-amine
20. (R)—N-(1-(3-amino-5-(trifluoromethyl)phenyl)ethyl)-2-chloro-6-(4-methoxypiperidin-1-yl)pyrido[3,4-d]pyrimidin-4-amine
21. (R)—N-(1-(3-amino-5-(trifluoromethyl)phenyl)ethyl)-2-chloro-6-(4,4-difluoropiperidin-1-yl)pyrido[3,4-d]pyrimidin-4-amine
22. (R)—N-(1-(3-amino-5-(trifluoromethyl)phenyl)ethyl)-2-chloro-6-(4-fluoropiperidin-1-yl)pyrido[3,4-d]pyrimidin-4-amine
23. (R)—N-(1-(3-amino-5-(trifluoromethyl)phenyl)ethyl)-2-chloro-6-(4-methylpiperazin-1-yl)pyrido[3,4-d]pyrimidin-4-amine
24. (R)—N-(1-(3-amino-5-(trifluoromethyl)phenyl)ethyl)-2-chloro-6-(4-ethylpiperazin-1-yl)pyrido[3,4-d]pyrimidin-4-amine
25. (R)—N-(1-(3-amino-5-(trifluoromethyl)phenyl)ethyl)-2-chloro-6-(4-isopropylpiperazin-1-yl)pyrido[3,4-d]pyrimidin-4-amine
26. (R)—N-(1-(3-amino-5-(trifluoromethyl)phenyl)ethyl)-2-chloro-6-(4-cyclopropylpiperazin-1-yl)pyrido[3,4-d]pyrimidin-4-amine
27. (R)—N-(1-(3-amino-5-(trifluoromethyl)phenyl)ethyl)-2-chloro-6-(4-methylpiperidin-1-yl)pyrido[3,4-d]pyrimidin-4-amine
28. (R)—N-(1-(3-amino-5-(trifluoromethyl)phenyl)ethyl)-2-chloro-6-(4-cyclobutylpiperazin-1-yl)pyrido[3,4-d]pyrimidin-4-amine
29. (R)-1-(4-((1-(3-amino-5-(trifluoromethyl)phenyl)ethyl)amino)-2-chloropyrido[3,4-d]pyrimidin-6-yl)piperidin-4-ol
30. (R)—N4-(1-(3-amino-5-(trifluoromethyl)phenyl)ethyl)-6-(4-methoxypiperidin-1-yl)-N2-methylpyrido[3,4-d]pyrimidine-2,4-diamine
31. (R)—N4-(1-(3-amino-5-(trifluoromethyl)phenyl)ethyl)-N2-methyl-6-(4-methylpiperazin-1-yl)pyrido[3,4-d]pyrimidine-2,4-diamine
32. (R)—N4-(1-(3-amino-5-(trifluoromethyl)phenyl)ethyl)-6-(4,4-difluoropiperidin-1-yl)-N2-methylpyrido[3,4-d]pyrimidine-2,4-diamine
33. (R)—N4-(1-(3-amino-5-(trifluoromethyl)phenyl)ethyl)-6-(4-fluoropiperidin-1-yl)-N2-methylpyrido[3,4-d]pyrimidine-2,4-diamine
34. (R)-1-(4-((1-(3-amino-5-(trifluoromethyl)phenyl)ethyl)amino)-6-(pyrrolidin-1-yl)pyrido[3,4-d]pyrimidin-2-yl)pyrrolidin-2-one
35. (R)—N4-(1-(3-amino-5-(trifluoromethyl)phenyl)ethyl)-N2-(2-(methylamino)ethyl)-6-morpholinopyrido[3,4-d]pyrimidine-2,4-diamine
36. (R)—N4-(1-(3-amino-5-(trifluoromethyl)phenyl)ethyl)-N2-(2-methoxyethyl)-6-morpholinopyrido[3,4-d]pyrimidine-2,4-diamine
37. (R)—N-(1-(3-amino-5-(trifluoromethyl)phenyl)ethyl)-2-(azetidin-1-yl)-6-morpholinopyrido[3,4-d]pyrimidin-4-amine
38. (R)—N4-(1-(3-amino-5-(trifluoromethyl)phenyl)ethyl)-N2-isopropyl-6-morpholinopyrido[3,4-d]pyrimidine-2,4-diamine
39. (R)—N4-(1-(3-amino-5-(trifluoromethyl)phenyl)ethyl)-N2-(2-(methylamino)ethyl)-6-(pyrrolidin-1-yl)pyrido[3,4-d]pyrimidine-2,4-diamine
40. (R)—N-(1-(3-amino-5-(trifluoromethyl)phenyl)ethyl)-2-(azetidin-1-yl)-6-(4-methoxypiperidin-1-yl)pyrido[3,4-d]pyrimidin-4-amine
41. (R)—N4-(1-(3-amino-5-(trifluoromethyl)phenyl)ethyl)-6-(4-methoxypiperidin-1-yl)-N2,N2-dimethylpyrido[3,4-d]pyrimidine-2,4-diamine
42. (R)—N4-(1-(3-amino-5-(trifluoromethyl)phenyl)ethyl)-N2-isopropyl-6-(4-methoxypiperidin-1-yl)pyrido[3,4-d]pyrimidine-2,4-diamine
43. (R)—N4-(1-(3-amino-5-(trifluoromethyl)phenyl)ethyl)-6-(4-fluoropiperidin-1-yl)-N2,N2-dimethylpyrido[3,4-d]pyrimidine-2,4-diamine
44. (R)—N-(1-(3-amino-5-(trifluoromethyl)phenyl)ethyl)-2-(azetidin-1-yl)-6-(4-fluoropiperidin-1-yl)pyrido[3,4-d]pyrimidin-4-amine
45. (R)—N4-(1-(3-amino-5-(trifluoromethyl)phenyl)ethyl)-N2,N2-dimethyl-6-(4-methylpiperazin-1-yl)pyrido[3,4-d]pyrimidine-2,4-diamine
46. (R)—N-(1-(3-amino-5-(trifluoromethyl)phenyl)ethyl)-2-(azetidin-1-yl)-6-(4-methylpiperazin-1-yl)pyrido[3,4-d]pyrimidin-4-amine
47. (R)—N-(1-(3-amino-5-(trifluoromethyl)phenyl)ethyl)-2-(azetidin-1-yl)-6-(4-methylpiperidin-1-yl)pyrido[3,4-d]pyrimidin-4-amine
48. (R)—N-(1-(3-amino-5-(trifluoromethyl)phenyl)ethyl)-2-(azetidin-1-yl)-6-(4-ethylpiperazin-1-yl)pyrido[3,4-d]pyrimidin-4-amine
49. (R)—N4-(1-(3-amino-5-(trifluoromethyl)phenyl)ethyl)-6-(4-ethylpiperazin-1-yl)-N2,N2-dimethylpyrido[3,4-d]pyrimidine-2,4-diamine
50. (R)—N4-(1-(3-amino-5-(trifluoromethyl)phenyl)ethyl)-6-(4-isopropylpiperazin-1-yl)-N2,N2-dimethylpyrido[3,4-d]pyrimidine-2,4-diamine
51. (R)—N-(1-(3-amino-5-(trifluoromethyl)phenyl)ethyl)-2-(azetidin-1-yl)-6-(4,4-difluoropiperidin-1-yl)pyrido[3,4-d]pyrimidin-4-amine
52. (R)—N-(1-(3-amino-5-(trifluoromethyl)phenyl)ethyl)-2-(azetidin-1-yl)-6-(4-cyclobutylpiperazin-1-yl)pyrido[3,4-d]pyrimidin-4-amine
53. (R)—N4-(1-(3-amino-5-(trifluoromethyl)phenyl)ethyl)-2-(azetidin-1-yl)-N6,N6-dimethylpyrido[3,4-d]pyrimidine-4,6-diamine
54. (R)—N4-(1-(3-amino-5-(trifluoromethyl)phenyl)ethyl)-2-(azetidin-1-yl)-N6-isopropylpyrido[3,4-d]pyrimidine-4,6-diamine
55. (R)—N4-(1-(3-amino-5-(trifluoromethyl)phenyl)ethyl)-N6-isopropyl-2-(pyrrolidin-1-yl)pyrido[3,4-d]pyrimidine-4,6-diamine
56. (R)—N4-(1-(3-amino-5-(trifluoromethyl)phenyl)ethyl)-2-(azetidin-1-yl)-N6-methylpyrido[3,4-d]pyrimidine-4,6-diamine
57. (R)—N-(1-(3-amino-5-(trifluoromethyl)phenyl)ethyl)-2-methoxy-6-morpholinopyrido[3,4-d]pyrimidin-4-amine
58. (R)—N-(1-(3-amino-5-(trifluoromethyl)phenyl)ethyl)-2-methoxy-6-(4-methoxypiperidin-1-yl)pyrido[3,4-d]pyrimidin-4-amine
59. (R)-2-chloro-N-(1-(3-(difluoromethyl)-2-fluorophenyl)ethyl)-6-morpholinopyrido[3,4-d]pyrimidin-4-amine
60. (R)-2-chloro-N-(1-(3-(difluoromethyl)-2-fluorophenyl)ethyl)-6-(4-methoxypiperidin-1-yl)pyrido[3,4-d]pyrimidin-4-amine
61. (R)-2-chloro-N-(1-(3-(difluoromethyl)-2-fluorophenyl)ethyl)-6-(4-methylpiperazin-1-yl)pyrido[3,4-d]pyrimidin-4-amine
62. (R)-2-chloro-N-(1-(3-(difluoromethyl)-2-fluorophenyl)ethyl)-6-(4-ethylpiperazin-1-yl)pyrido[3,4-d]pyrimidin-4-amine
63. (R)—N-(1-(3-amino-5-(trifluoromethyl)phenyl)ethyl)-2-chloro-6-(tetrahydro-2H-pyran-4-yl)pyrido[3,4-d]pyrimidin-4-amine
64. (R)-2-chloro-N-(1-(3-(difluoromethyl)-2-fluorophenyl)ethyl)-6-(tetrahydro-2H-pyran-4-yl)pyrido[3,4-d]pyrimidin-4-amine
65. (R)—N4-(1-(3-amino-5-(trifluoromethyl)phenyl)ethyl)-N2,N2-dimethyl-6-(tetrahydro-2H-pyran-4-yl)pyrido[3,4-d]pyrimidine-2,4-diamine
66. (R)—N4-(1-(3-amino-5-(trifluoromethyl)phenyl)ethyl)-N2-methyl-6-(tetrahydro-2H-pyran-4-yl)pyrido[3,4-d]pyrimidine-2,4-diamine
67. (R)—N-(1-(3-amino-5-(trifluoromethyl)phenyl)ethyl)-2-(azetidin-1-yl)-6-(tetrahydro-2H-pyran-4-yl)pyrido[3,4-d]pyrimidin-4-amine
68. (R)—N-(1-(3-amino-5-(trifluoromethyl)phenyl)ethyl)-2-(azetidin-1-yl)-6-(1-methylpiperidin-4-yl)pyrido[3,4-d]pyrimidin-4-amine
69. (R)—N4-(1-(3-amino-5-(trifluoromethyl)phenyl)ethyl)-N2,N2-dimethyl-6-(1-methylpiperidin-4-yl)pyrido[3,4-d]pyrimidine-2,4-diamine
70. (R)—N-(1-(3-amino-5-(trifluoromethyl)phenyl)ethyl)-2-chloro-6-(4-ethylpiperazin-1-yl)-7-fluoroquinazolin-4-amine
71. (R)—N-(1-(3-amino-5-(trifluoromethyl)phenyl)ethyl)-2-chloro-6-(4-ethylpiperazin-1-yl)-7-methylquinazolin-4-amine
72. (R)—N-(1-(3-amino-5-(trifluoromethyl)phenyl)ethyl)-2-chloro-6-(4-ethylpiperazin-1-yl)-7-methoxyquinazolin-4-amine
73. (R)—N-(1-(3-amino-5-(trifluoromethyl)phenyl)ethyl)-2-chloro-6-(4-ethylpiperazin-1-yl)quinazolin-4-amine
74. (R)—N4-(1-(3-amino-5-(trifluoromethyl)phenyl)ethyl)-N2-methyl-6-morpholinoquinazoline-2,4-diamine
75. (R)—N-(4-((1-(3-amino-5-(trifluoromethyl)phenyl)ethyl)amino)-6-(pyrrolidin-1-yl)pyrido[3,4-d]pyrimidin-2-yl)propionamide
76. (R)—N-(4-((1-(3-amino-5-(trifluoromethyl)phenyl)ethyl)amino)-6-(pyrrolidin-1-yl)pyrido[3,4-d]pyrimidin-2-yl)-2-methoxyacetamide
77. (R)-2-amino-N-(4-((1-(3-amino-5-(trifluoromethyl)phenyl)ethyl)amino)-6-(pyrrolidin-1-yl)pyrido[3,4-d]pyrimidin-2-yl)acetamide hydro chloride
78. (R)—N-(4-((1-(3-amino-5-(trifluoromethyl)phenyl)ethyl)amino)-6-(pyrrolidin-1-yl)pyrido[3,4-d]pyrimidin-2-yl)-3-(methylamino)propenamide
79. (R)—N-(4-((1-(3-amino-5-(trifluoromethyl)phenyl)ethyl)amino)-6-morpholinopyrido[3,4-d]pyrimidin-2-yl)-3-(methylamino)propanamide
80. (R)—N-(1-(3-amino-5-(trifluoromethyl)phenyl)ethyl)-2-chloro-6-(4-ethylpiperazin-1-yl)pyrido[2,3-d]pyrimidin-4-amine
81. (R)—N-(1-(3-amino-5-(trifluoromethyl)phenyl)ethyl)-6-(4-ethylpiperazin-1-yl)-2-methoxypyrido[2,3-d]pyrimidin-4-amine
82. (R)—N-(1-(3-amino-5-(trifluoromethyl)phenyl)ethyl)-2-chloro-6-(4-methylpiperazin-1-yl)pyrido[2,3-d]pyrimidin-4-amine
83. (R)—N-(1-(3-amino-5-(trifluoromethyl)phenyl)ethyl)-2-chloro-6-(piperidin-1-yl)pyrido[2,3-d]pyrimidin-4-amine
84. (R)—N-(1-(3-amino-5-(trifluoromethyl)phenyl)ethyl)-2-chloro-6-morpholinopyrido[2,3-d]pyrimidin-4-amine
85. (R)—N-(1-(3-amino-5-(trifluoromethyl)phenyl)ethyl)-6-(4-ethylpiperazin-1-yl)pyrido[3,4-c]pyridazin-4-amine
86. (R)—N-(1-(3-amino-5-(trifluoromethyl)phenyl)ethyl)-3-(4-ethylpiperazin-1-yl)-8-methylpyrido[2,3-d]pyridazin-5-amine
87. (R)—N-(1-(3-amino-5-(trifluoromethyl)phenyl)ethyl)-8-methyl-3-morpholinopyrido[2,3-d]pyridazin-5-amine
88. (R)—N-(1-(3-amino-5-(trifluoromethyl)phenyl)ethyl)-7-chloro-3-(4-ethylpiperazin-1-yl)-1,6-naphthyridin-5-amine
89. (R)—N-(1-(3-amino-5-(trifluoromethyl)phenyl)ethyl)-7-chloro-3-morpholino-1,6-naphthyridin-5-amine
90. N4-((R)-1-(3-amino-5-(trifluoromethyl)phenyl)ethyl)-N2-methylsulfonyl(pyrrolidin-1-yl)pyrido[3,4-d]pyrimidine-2,4-diamine
91. N4-((R)-1-(3-amino-5-(trifluoromethyl)phenyl)ethyl)-N2-aminosulfonyl(pyrrolidin-1-yl)pyrido[3,4-d]pyrimidine-2,4-diamine
92. N4-((R)-1-(3-amino-5-(trifluoromethyl)phenyl)ethyl)-N6-methyl-2-(pyrrolidin-1-yl)pyrido[3,4-d]pyrimidine-4,6-diamine
93. N4-((R)-1-(3-amino-5-(trifluoromethyl)phenyl)ethyl)-6-cyclopropyl-N2-methylpyrido[3,4-d]pyrimidine-2,4-diamine
94. N4-((R)-1-(3-amino-5-(trifluoromethyl)phenyl)ethyl)-6-cyclobutyl-N2-methylpyrido[3,4-d]pyrimidine-2,4-diamine
95. N-(4-((R)-1-(3-amino-5-(trifluoromethyl)phenyl)ethylamino)-6-(pyrrolidin-1-yl)pyrido[3,4-d]pyrimidin-2-yl)-2-(methylamino) 2-aminoacetamide
96. N-(4-((R)-1-(3-amino-5-(trifluoromethyl)phenyl)ethylamino)-6-(pyrrolidin-1-yl)pyrido[3,4-d]pyrimidin-2-yl)-3-(methylamino)propanamide
97. 4-(4-((R)-1-(3-amino-5-(trifluoromethyl)phenyl)ethylamino)-2-(methylamino)pyrido[3,4-d]pyrimidin-6-yl)morpholin-3-one
98. 1-(4-((R)-1-(3-amino-5-(trifluoromethyl)phenyl)ethylamino)-2-(methylamino)pyrido[3,4-d]pyrimidin-6-yl)pyrrolidin-2-one
99. 4-(4-((R)-1-(3-amino-5-(trifluoromethyl)phenyl)ethylamino)-2-(methylamino)pyrido[3,4-d]pyrimidin-6-yl)morpholin-2-one
100. 1-(4-((R)-1-(3-amino-5-(trifluoromethyl)phenyl)ethylamino)-2-(methylamino)pyrido[3,4-d]pyrimidin-6-yl)piperidin-4-ol
101. N4-((R)-1-(4-(trifluoromethyl)thiophen-2-yl)ethyl)-N2-methyl-6-morpholinopyrido[3,4-d]pyrimidine-2,4-diamine
102. N4-((R)-1-(3-fluoro-4-(difluoromethyl)thiophen-2-yl)ethyl)-N2-methyl-6-morpholinopyrido[3,4-d]pyrimidine-2,4-diamine
103. 1-(4-((R)-1-(3-amino-5-(trifluoromethyl)phenyl)ethylamino)-6-(methylamino)pyrido[3,4-d]pyrimidin-2-yl)pyrrolidin-2-one
104. N—((R)-1-(3-amino-5-(trifluoromethyl)phenyl)ethyl)-6-methoxy-2-(pyrrolidin-1-yl)pyrido[3,4-d]pyrimidin-4-amine
105. (R)-1-(4-(4-((1-(3-amino-5-(trifluoromethyl)phenyl)ethyl)amino)-2-chloropyrido[2,3-d]pyrimidin-6-yl)piperazin-1-yl)ethan-1-one
106. (R)—N-(1-(3-amino-5-(trifluoromethyl)phenyl)ethyl)-2-chloro-6-(4-ethylpiperazin-1-yl)-7-methylpyrido[2,3-d]pyrimidin-4-amine
107. (R)-2-(4-(4-((1-(3-amino-5-(trifluoromethyl)phenyl)ethyl)amino)-2-chloropyrido[2,3-d]pyrimidin-6-yl)piperazin-1-yl)ethan-1-ol
108. (R)—N-(1-(3-amino-5-(trifluoromethyl)phenyl)ethyl)-2-chloro-6-(4-(2-methoxyethyl)piperazin-1-yl)pyrido[2,3-d]pyrimidin-4-amine
109. (R)—N-(1-(3-amino-5-(trifluoromethyl)phenyl)ethyl)-2-chloro-6-(tetrahydro-2H-pyran-4-yl)pyrido[2,3-d]pyrimidin-4-amine
110. (R)—N-(1-(3-amino-5-(trifluoromethyl)phenyl)ethyl)-3-(4-ethylpiperazin-1-yl)-8-fluoropyrido[2,3-d]pyridazin-5-amine
111. (R)-4-((1-(3-amino-5-(trifluoromethyl)phenyl)ethyl)amino)-2-chloro-6-(4-ethylpiperazin-1-yl)-7-methylpyrido[3,4-d]pyrimidin-8(7H)-one
112. (R)-4-((1-(3-amino-5-(trifluoromethyl)phenyl)ethyl)amino)-6-(4-ethylpiperazin-1-yl)-2,7-dimethylpyrido[3,4-d]pyrimidin-8(7H)-one
113. (R)—N-(1-(3-amino-5-(trifluoromethyl)phenyl)ethyl)-3-(4-ethylpiperazin-1-yl)-1,7-naphthyridin-5-amine
114. (R)—N-(1-(3-amino-5-(trifluoromethyl)phenyl)ethyl)-6-(4-ethylpiperazin-1-yl)-2,7-naphthyridin-4-amine
115. (R)—N-(1-(3-amino-5-(trifluoromethyl)phenyl)ethyl)-2-chloro-6-morpholino-1,8-naphthyridin-4-amine
116. (R)—N-(1-(3-amino-5-(trifluoromethyl)phenyl)ethyl)-2-methyl-6-morpholino-1,8-naphthyridin-4-amine
117. (R)—N-(1-(3-amino-5-(trifluoromethyl)phenyl)ethyl)-3-(4-ethylpiperazin-1-yl)-8-methyl-1,7-naphthyridin-5-amine
118. (R)—N-(1-(3-amino-5-(trifluoromethyl)phenyl)ethyl)-3-(4-ethylpiperazin-1-yl)-8-methyl-1,6-naphthyridin-5-amine
119. (R)-1-((1-(3-amino-5-(trifluoromethyl)phenyl)ethyl)amino)-7-(4-ethylpiperazin-1-yl)-6-methylpyrido[3,4-d]pyridazin-5(6H)-one
120. (R)-1-((1-(3-amino-5-(trifluoromethyl)phenyl)ethyl)amino)-7-(4-ethylpiperazin-1-yl)-4,6-dimethylpyrido[3,4-d]pyridazin-5(6H)-one
121. (R)-1-((1-(3-amino-5-(trifluoromethyl)phenyl)ethyl)amino)-7-(4-ethylpiperazin-1-yl)-4-fluoro-6-methylpyrido[3,4-d]pyridazin-5(6H)-one
122. (R)—N-(1-(3-amino-5-(trifluoromethyl)phenyl)ethyl)-2-chloro-6-(1-ethylpiperidin-4-yl)-7-methyl-6,7-dihydropyrimido[4,5-d]pyridazin-4-amine
   or a pharmaceutically acceptable salt thereof or a pharmaceutically acceptable regioisomer thereof.

Further embodiments of the invention includes use of compounds of formula (I) or pharmaceutically acceptable derivatives, salts and regio-isomers thereof, including mixtures thereof in all ratios as a medicament.

Use of compounds as above and pharmaceutically usable derivatives, salts and regioisomers thereof, including mixtures thereof in all ratios, for the preparation of a medicament for the treatment of cancer and other associated disorders.

Targeting SOS1 appears promising for treating cancer and various other non-cancerous indications like RASopathies including neurofibromatosis (NF1), Costello syndrome (CS), Legius (LGSS), Leopard (LPRD), cardiofaciocutaneous syndrome (CFC), Noonan syndrome (NS) and other NS-like disorders (Mol. Syndromology, 2010, 1, 2-26; Annu. Rev. Genomics. Human. Genetics, 2013, 14, 355-369; Human Mol. Genetics, 2016, 25, R¹²³—R¹²⁵; Curr. Gen. Med. Rep, 2016, 4, 57-64; BBA—Reviews on Cancer 1874 (2020) 188445). Noonan syndrome is an autosomal dominant disease bearing SOS1 gain of function mutations. SOS1 involvement in hereditary gingival fibromatosis is also reported. HGF is a rare, autosomal dominant condition characterized by benign, uncontrolled gingival overgrowth. The causal mutation in SOS1 gene results in lack of autoinhibitory domain of the protein leading to constitutive activation and uncontrolled growth in the gingival region (J Dent Res, 2007, 86, 25-34; Am. J. Human Genetics, 2002, 70, 943-954).

Unless specifically indicated, the general formula of compound (I) shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

The present invention further provides a pharmaceutical composition comprising at least one compound according to formula (I) and/or pharmaceutically usable derivatives, salts, tautomers and regioisomers thereof, including mixtures thereof in all ratios, optional additional second active ingredient, and excipients.

The term “pharmaceutically acceptable salt” or “pharmaceutically acceptable derivatives” is taken to mean an active ingredient, which comprises a compound of the formula (I) in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The term “regioisomer” or “regioisomers” refers to the positional isomers, which is a category of structural isomers, wherein the position or the substituent changes position on the parent structure. Herein the term regioisomer without departing from the scope of compound of formula (I) inherently includes all regioisomers either as a pure regioisomer or mixture of two or more regioisomers thereof. Since the pharmaceutical activity of the regioisomers of the compounds of the present invention may differ, it may be desirable to use the regioisomers. In these cases the regioisomers can be separated at any of the possible stage either as an intermediate or as an end product by the process well known to the person skilled in the art or even employed as such in the synthesis.

The term “tautomer” or “tautomers” refers to the compound of formula (I) of the present invention wherein any hydrogen atom is replaced by a hydroxyl group on a carbon with a double bond. The present invention includes all possible tautomeric forms.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

For example, in the case of oral administration as tablet or capsule, the active-ingredient component can be combined with an oral, non-toxic and pharmaceutically acceptable inert excipient, such as, for example, ethanol, glycerol, water and the like.

Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form can be added to the powder mixture before the filling operation. A disintegrant or solubiliser, such as, for example, agar-agar, calcium carbonate or sodium carbonate, may likewise be added in order to improve the availability of the medica-ment after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The active ingredients can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compounds. Syrups can be prepared by dissolving the compounds in an aqueous solution with a suitable flavour, while elixirs are prepared using a non-toxic alcoholic vehicle. Suspensions can be for-mulated by dispersion of the compounds in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The formulations can be in the form of liposome delivery systems, such as, for example, small unilamellar vesicles, large unilamellar vesicles and multilamellar vesicles. Liposomes can be formed from suitable lipids or phospholipids or both, such as, for example, cholesterol, stearylamine or phosphatidylcholines or the like.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurized dispensers with aerosols, nebulisers or inhalers.

Pharmaceutical formulations adapted for vaginal administration can be administered as pessaries, tampons, creams, gels, pastes, foams or spray formulations. Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula (I) and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

In a further aspect, the present invention relates to a process for preparing novel compounds of formula (I).

The novel compounds of formula (I) may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures. Moreover, by utilizing the procedures described in detail, one of ordinary skill in the art can prepare additional compounds of the present invention claimed herein. All temperatures are in degrees Celsius (° C.) unless otherwise noted.

In another embodiment of the present invention provides methods useful for preparing the compounds of formula (I) depicted generically in the Schemes given hereunder. One skilled-in-the-art will recognize that any of the Schemes can be adapted to produce the compounds of formula (I) and pharmaceutically accepted salts of compounds of formula (I) according to the present invention. All symbols/variables are as defined hereunder unless otherwise stated.

Compounds of this invention can be isolated in association with solvent molecules by crystallization from evaporation of an appropriate solvent. The pharmaceutically acceptable acid addition salts of the compounds of formula (I), which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of formula (I) with a suitable base. Both types of salts may be formed or interconverted using ion-exchange resin techniques.

### EXAMPLES

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

### Synthesis of (R)-2-chloro-N-(1-(3-nitro-5-(trifluoromethyl)phenyl)ethyl)-6-(pyrrolidin-1-yl)pyrido[3,4-d]pyrimidin-4-amine (intermediate 1f)

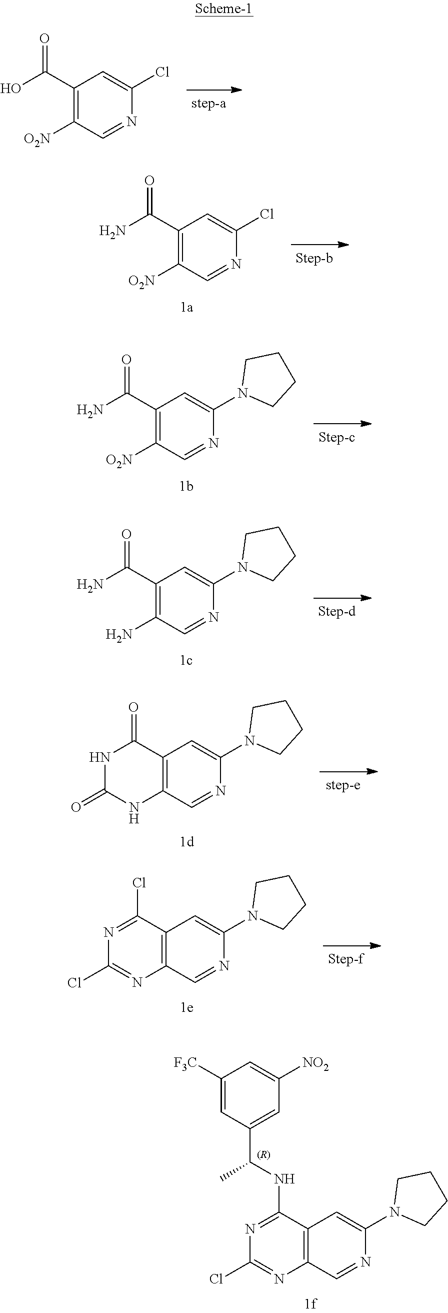

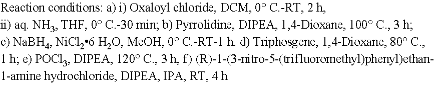

Step—a: 2-Chloro-5-nitroisonicotinamide (1a): To a solution 2-chloro-5-nitroisonicotinic acid (9.0 g, 44.43 mmol) in dry dichloromethane (450 mL) was added N,N-dimethylformamide (5 drops) under nitrogen atmosphere. The reaction mixture was cooled to 0° C. and oxalyl chloride (17.0 g, 133.94 mmol) was added drop wise. The solution was warmed to room temperature and stirred for 2 h. The reaction mixture was distilled under reduced pressure and residue was co-distilled with dichloromethane (2×20 mL). The residue was dissolved in dry tetrahydrofuran (450 mL) and ammonia gas was purged into it for 30 minutes. Reaction progress was monitored by TLC. The solid obtained was filtered and washed with ethyl acetate and the combined filtrate was evaporated under reduced pressure. The solid formed was triturated with 25% ethyl acetate in hexane to afford the title compound as pale-yellow solid. Yield: 7.0 g (77%); ¹H-NMR (300 MHz, DMSO-d₆): δ 9.12 (s, 1H), 8.33 (bs, 1H), 8.08 (bs, 1H), 7.90 (s, 1H); LCMS: m/z 201.1 [M−H].

Step—b: 5-Nitro-2-(pyrrolidin-1-yl)isonicotinamide (1b): To a solution of 2-chloro-5-nitroisonicotinamide (3.5 g, 17.36 mmol) in 1,4-dioxane (140 mL) were added diisopropylethylamine (10.5 mL, 52.09 mmol) and pyrrolidine (7 mL, 86.81 mmol) under nitrogen atmosphere at room temperature. The reaction mixture was heated at 90° C. and stirred for 16 h. Reaction progress was monitored by TLC. After completion of reaction, 1,4-dioxane was distilled off under reduced pressure. The solid obtained was filtered, washed with water and dried under vacuum to afford the title compound as a yellow solid. Yield: 3.3 g (80%); ¹H-NMR (300 MHz, DMSO-d₆): δ 8.88 (s, 1H), 7.94 (bs, 1H), 7.67 (bs, 1H), 6.43 (s, 1H), 3.47 (bs, 4H), 1.97 (bs, 4H); LCMS: m/z 237.2 [M+H]⁺.

Step—c: 5-Amino-2-(pyrrolidin-1-yl)isonicotinamide (1c): To a stirred solution of 5-nitro-2-(pyrrolidin-1-yl)isonicotinamide (2.0 g, 8.47 mmol) in MeOH was added NiCl₂.6H₂0 (0.80 g, 3.38 mmol) at RT and stirred for 5 min at same temperature. NaBH₄ (1.28 g, 33.83 mmol) was added portion wise at 0° C. and stirred for 1 h at RT. Reaction progress was monitored by TLC. The reaction mixture was quenched with sat. NaHCO₃ solution and filtered under vacuum. The filtrate was extracted with DCM (4×100 ml). Combined organic layers were washed with brine solution, dried over Na₂SO₄, concentrated to give the crude product which was purified by flash column chromatography using 2-3% MeOH in DCM as an eluent to afford the title compound as a brown solid (Yield: 1.50 g, 86%); ¹H-NMR (300 MHz, DMSO-d₆): δ 7.99 (bs, 1H), 7.74 (s, 1H), 7.40 (bs, 1H), 7.03 (bs, 1H), 6.53 (s, 1H), 6.41 (bs, 1H), 3.32-3.26 (m, 4H), 1.92-1.88 (m, 4H); LCMS: m/z 207.1 [M+H]⁺.

Step—d: 6-(Pyrrolidin-1-yl)pyrido[3,4-d]pyrimidine-2,4(1H,3H)-dione (ld): 5-Amino-2-(pyrrolidin-1-yl)isonicotinamide (1.50 g, 7.28 mmol) was dissolved in 1,4-dioxane (15 mL) by sonication. To the above solution at room temperature was added triphosgene (4.32 g, 14.56 mmol) in portions. The reaction mixture was stirred at 80° C. for 1 h and the reaction progress was monitored by TLC. Upon consumption of starting material, 1,4-dioxane was distilled off under reduced pressure and the residue was triturated with ethyl acetate. The resulting solid was filtered, washed with ethyl acetate and dried under vacuum to afford the title compound as a yellow solid. Yield: 1.40 g (82%); ¹H-NMR (300 MHz, DMSO-d₆): δ 11.40 (s, 1H), 10.92 (s, 1H), 8.10 (s, 1H), 6.84 (s, 1H), 3.41 (bs, 4H), 1.96 (bs, 4H); LCMS: m/z 233.1 [M+H]⁺.

Step—e: 2,4-Dichloro-6-(pyrrolidin-1-yl)pyrido[3,4-d]pyrimidine (le): To a solution of 6-(pyrrolidin-1-yl)pyrido[3,4-d]pyrimidine-2,4(1H,3H)-dione (1.4 g, 6.03 mmol) in phosphorus(V) oxychloride (14 mL) was added n,n-diisopropylethylamine (3.21 mL, 18.09 mmol) at RT and the reaction mixture was stirred at 120° C. for 3 h. The progress of the reaction was monitored by TLC. Upon completion of the reaction, phosphorus(V)oxychloride was distilled off under reduced pressure. Obtained residue was washed with diethyl ether (4×30 mL), the combined organic layers were washed with saturated sodium bicarbonate solution (50 mL), brine solution (50 mL), dried over anhydrous sodium sulfate and concentrated under reduced pressure to afford the title compound as orange solid. Yield: 1.0 g (61%); ¹H-NMR (300 MHz, CDCl₃): δ 9.06 (s, 1H), 6.51 (s, 1H), 3.58 (t, J=6.3 Hz, 4H), 2.13-2.08 (m, 4H); LCMS: m/z 269.2 [M+H]⁺.

Step—f: (R)-2-Chloro-N-(1-(3-nitro-5-(trifluoromethyl)phenyl)ethyl)-6-(pyrrolidin-1-yl)pyrido[3,4-d]pyrimidin-4-amine (if): To a solution of 2,4-dichloro-6-(pyrrolidin-1-yl)pyrido[3,4-d]pyrimidine (1.0 g, 3.73 mmol) in IPA (20 mL) were added DIPEA (2.0 mL, 11.19 mmol) and (R)-1-(3-nitro-5-(trifluoromethyl)phenyl)ethan-1-amine hydrochloride (1.10 g, 4.10 mmol). The reaction mixture was stirred at room temperature for 4 h. Reaction progress was monitored by TLC. After completion of reaction, it was quenched with water (30 mL), extracted with EtOAc (3×50 mL). Separated organic layers were washed with brine (50 mL), dried over anhydrous sodium sulfate and concentrated under reduced pressure to provide the crude product which was purified by flash column chromatography and eluted with 20-25% ethyl acetate in hexane to afford the title compound as a brown solid. Yield 1.50 g (86%); ¹H-NMR (300 MHz, CDCl₃): δ 8.84 (s, 1H), 8.50 (s, 1H), 8.41 (s, 1H), 8.10 (s, 1H), 6.12 (s, 1H), 5.99 (d, J=5.7 Hz, 1H), 5.64 (quin, J=6.9 Hz, 1H), 3.55 (t, J=6.6 Hz, 4H), 2.11-2.06 (m, 4H), 1.80 (d, J=7.2 Hz, 3H); LCMS: m/z 467 [M+H]⁺.

### Example—1: (R)—N-(1-(3-amino-5-(trifluoromethyl)phenyl)ethyl)-2-chloro-6-(pyrrolidin-1-yl)pyrido[3,4-d]pyrimidin-4-amine

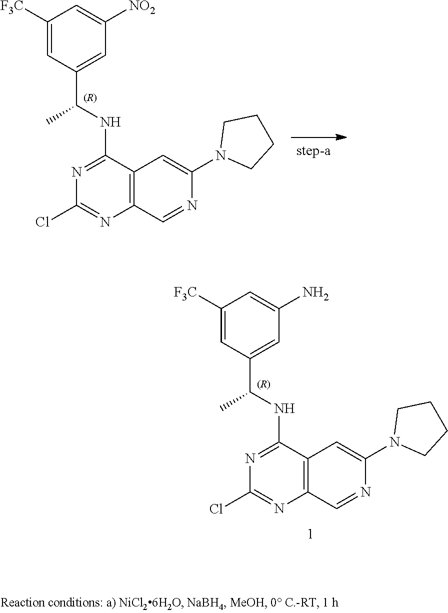

To a stirred solution of (R)-2-chloro-N-(1-(3-nitro-5-(trifluoromethyl)phenyl)ethyl)-6-(pyrrolidin-1-yl)pyrido[3,4-d]pyrimidin-4-amine (50 mg, 0.11 mmol) in MeOH was added NiCl₂.6H₂0 (5.2 mg, 0.022 mmol) at RT and stirred for 5 min at same temperature. NaBH₄ (17 mg, 0.44 mmol) was added portion wise at 0° C. and stirred for 1 h at RT. Reaction progress was monitored by TLC. The reaction mixture was quenched with saturated NaHCO₃ solution and filtered under vacuum. The filtrate was extracted with DCM (4×20 ml) and combined organic layers were washed with brine solution, dried over Na₂SO₄, concentrated to get crude product which was purified by flash column chromatography using 2-3% MeOH in DCM as an eluent to afford the title compound as a brown solid (Yield: 20 mg, 43%); ¹H-NMR (400 MHz, DMSO-d₆): δ 8.85 (d, J=7.7 Hz, 1H), 8.63 (s, 1H), 6 7.36 (s, 1H), 7.10 (s, 1H), 6.85 (s, 1H), 6.72 (s, 1H), 5.59 (bs, 2H), 5.44 (t, J=7 Hz, 1H), 3.47 (bs, 4H), 2.0 (bs, 4H), 1.58 (d, J=6.8 Hz, 3H); LCMS: m/z 437.10 [M+H]⁺.

### Example—2: N4-(R)-(1-(3-amino-5-(trifluoromethyl)phenyl)ethyl)-N2,N2-dimethyl-6-(pyrrolidin-1-yl)pyrido[3,4-d]pyrimidine-2,4-diamine

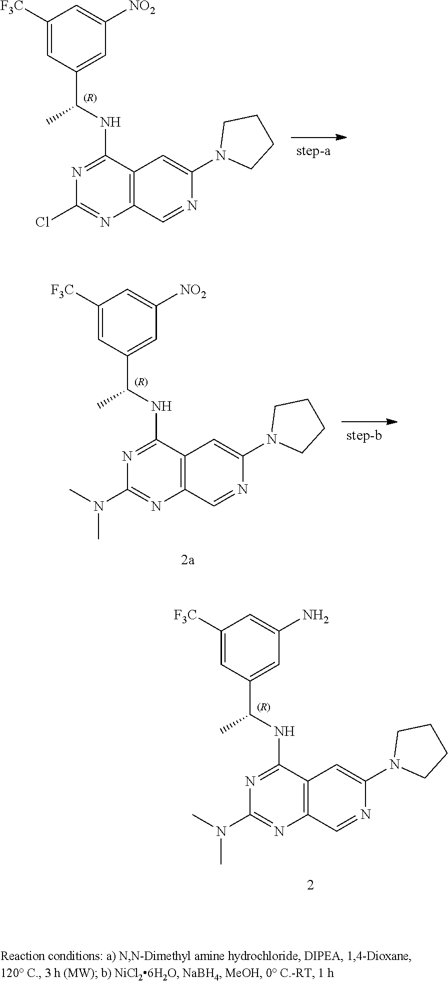

### Step—a: (R)—N2,N2-dimethyl-N4-(1-(3-nitro-5-(trifluoromethyl)phenyl)ethyl)-6-(pyrrolidin-1-yl)pyrido[3,4-d]pyrimidine-2,4-diamine (2a)

To a stirred solution of (R)-2-chloro-N-(1-(3-nitro-5-(trifluoromethyl)phenyl)ethyl)-6-(pyrrolidin-1-yl)pyrido[3,4-d]pyrimidin-4-amine (100 mg, 0.21 mmol) in 1,4-Dioxane (5 mL) in microwave vial were added N,N-diisopropylethylamine (0.19 mL) and N,N dimethylamine hydrochloride (0.034 g. 0.42 mmol) at RT. The reaction vial was placed in Microwave reactor and irradiated at 120° C. for 3 hr. The reaction mixture was quenched with water (20 mL), extracted with DCM (3×20 ml). The combined organic layers were washed with brine (20 mL), dried over Na₂SO₄, concentrated to get crude product which was purified by flash column chromatography using 1-2% MeOH in DCM as an eluent to afford the title compound as a yellow solid (yield: 50 mg, 50%); ¹H-NMR (400 MHz, DMSO-d₆): δ 8.56 (s, 1H), 8.40 (d, J=6.8 Hz, 1H), 8.35 (s, 1H), 8.34 (d, J=8.3 Hz, 2H), 6.94 (s, 1H), 5.4 (t, J=6.8 Hz, 1H), 3.44 (d, J=4.8 Hz, 4H), 2.95 (s, 6H), 1.99 (d, J=6.2 Hz, 4H), 1.66 (d, J=7.0 Hz, 3H); LCMS: m/z 476.30 [M+H]⁺.

### Step—b: Synthesis of (R)—N4-(1-(3-amino-5-(trifluoromethyl)phenyl)ethyl)-N2,N2-dimethyl-6-(pyrrolidin-1-yl)pyrido[3,4-d]pyrimidine-2,4-diamine (2)

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 50%; ¹H-NMR (400 MHz, DMSO-d₆): δ 8.4 (bs, 1H), 7.05 (bs, 1H), 6.85 (s, 1H), 6.80 (s, 1H), 6.68 (s, 1H), 5.54 (bs, 2H), 5.30 (t, J=6.8 Hz, 1H), 3.43 (d, J=3.6 Hz, 4H), 3.03 (s, 6H), 1.98 (s, 4H), 1.57 (d, J=6.8 Hz, 3H), One exchangeable proton not observed in NMR; LCMS: m/z 446.20 [M+H]⁺.

### Example—3: (R)—N-(1-(3-amino-5-(trifluoromethyl)phenyl)ethyl)-2-methoxy-6-(pyrrolidin-1-yl)pyrido[3,4-d]pyrimidin-4-amine

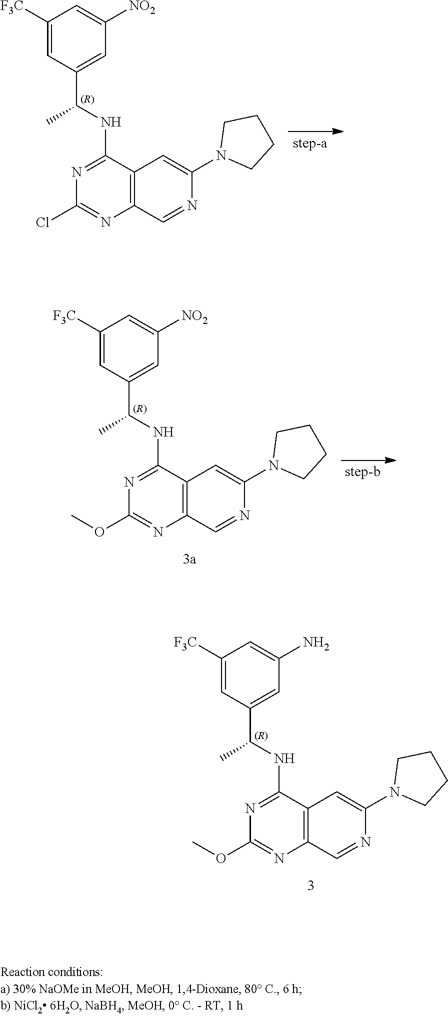

### Step—a: Synthesis of (R)-2-methoxy-N-(1-(3-nitro-5-(trifluoromethyl)phenyl)ethyl)-6-(pyrrolidin-1-yl)pyrido[3,4-d]pyrimidin-4-amine (3a)

To a stirred solution of (R)-2-chloro-N-(1-(3-nitro-5-(trifluoromethyl)phenyl)ethyl)-6-(pyrrolidin-1-yl)pyrido[3,4-d]pyrimidin-4-amine (90 mg, 0.19 mmol) in dry MeOH (2 mL) in sealed tube was added 30% of sodium methoxide in methanol solution (1 mL). The reaction mixture was capped and stirred at 80° C. for 6 h. Progress of the reaction was monitored by TLC. Reaction mixture was evaporated under reduced pressure and the residue obtained was dissolved in DCM (20 mL) and washed with water. Separated aqueous layer was extracted with DCM (2×20 mL). The combined organic layers were washed with brine (20 mL), dried over Na₂SO₄, concentrated to get crude product which was purified by flash column chromatography using 50-60% EtOAc in hexane as an eluent to afford the title compound as a yellow solid (Yield: 40 mg, 45%); LCMS: m/z 463.20 [M+H]⁺.

Step—b: Synthesis of (R)—N-(1-(3-amino-5-(trifluoromethyl)phenyl)ethyl)-2-methoxy-6-(pyrrolidin-1-yl)pyrido[3,4-d]pyrimidin-4-amine (3) The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 90%. ¹H-NMR (400 MHz, DMSO-d₆): δ 8.53 (s, 1H), 8.48 (d, J=7.7 Hz, 1H), 7.10 (s, 1H), 6.83 (s, 1H), 6.79 (s, 1H), 6.70 (s, 1H), 5.56 (bs, 2H), 5.41 (t, J=6.9 Hz, 1H), 3.77 (s, 3H), 3.45 (d, J=2.8 Hz, 4H), 1.99 (bs, 4H), 1.56 (d, J=7.1 Hz, 3H); LCMS: m/z 433.20 [M+H]⁺.

### Example—4: (R)—N-(1-(3-amino-5-(trifluoromethyl)phenyl)ethyl)-2-(2-methoxyethoxy)-6-(pyrrolidin-1-yl)pyrido[3,4-d]pyrimidin-4-amine

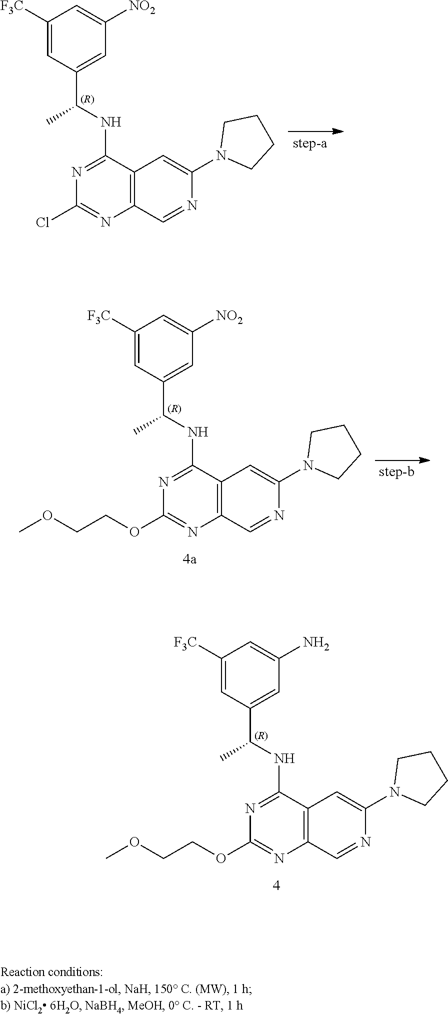

### Step—a: (R)-2-(2-methoxyethoxy)-N-(1-(3-nitro-5-(trifluoromethyl)phenyl)ethyl)-6-(pyrrolidin-1-yl)pyrido[3,4-d]pyrimidin-4-amine (4a)

To a stirred solution of (R)-2-chloro-N-(1-(3-nitro-5-(trifluoromethyl)phenyl)ethyl)-6-(pyrrolidin-1-yl)pyrido[3,4-d]pyrimidin-4-amine (100 mg, 0.214 mmol) in 2-methoxyethan-1-ol (2 mL) in microwave vial was added NaH-56% (30 mg,0.322 mmol) at RT and the reaction mixture was placed in microwave reactor and irradiated at 150° C. for 1 hr. Reaction mixture was quenched with water, extracted with DCM (3×20 ml). The combined organic layers were washed with brine, dried over Na₂SO₄, concentrated and the crude obtained was purified by flash column chromatography using 2-3% MeOH in DCM as an eluent to afford the title compound as a yellow solid (Yield:20 mg, 18%); LCMS: m/z 507.30 [M+H]⁺.

### Step—b: Synthesis of (R)—N-(1-(3-amino-5-(trifluoromethyl)phenyl)ethyl)-2-(2-methoxyethoxy)-6-(pyrrolidin-1-yl)pyrido[3,4-d]pyrimidin-4-amine (4)

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 52%; ¹H-NMR (400 MHz, DMSO-d₆): δ 8.51 (s, 1H), 8.50 (d, J=7.2 Hz, 1H), 7.11 (s, 1H), 6.83 (s, 1H), 6.80 (s, 1H), 6.70 (s, 1H), 5.5 (s, 2H), 5.45 (t, J=7.1 Hz, 1H), 4.31-4.26 (m, 2H), 3.58-3.56 (m, 2H), 3.45 (d, J=3.1 Hz, 4H), 3.26 (s, 3H), 1.99 (bs, 4H), 1.56 (d, J=6.8 Hz, 3H); LCMS: m/z 477.20 [M+H]⁺.

### Example—5: (R)—N-(1-(3-amino-5-(trifluoromethyl)phenyl)ethyl)-2-morpholino-6-(pyrrolidin-1-yl)pyrido[3,4-d]pyrimidin-4-amine

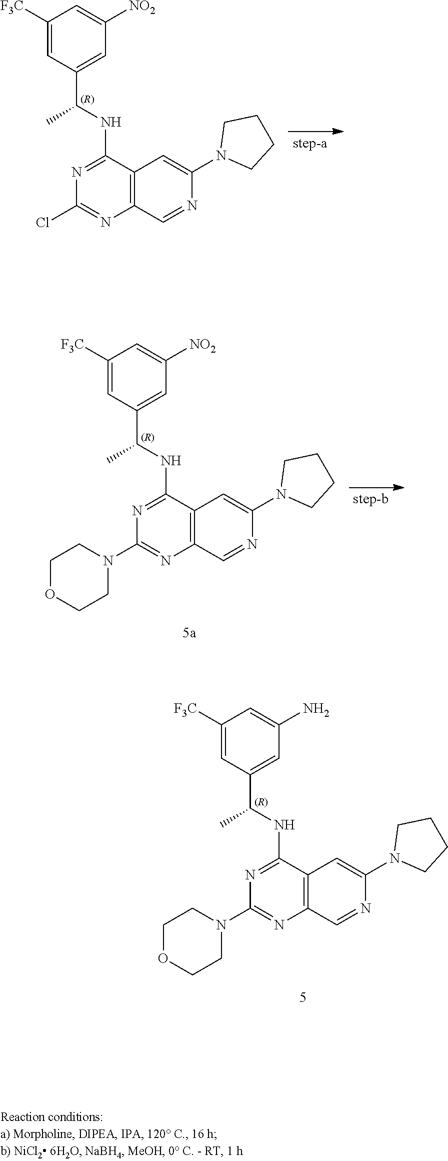

### Step—a: Synthesis of (R)-2-morpholino-N-(1-(3-nitro-5-(trifluoromethyl)phenyl)ethyl)-6-(pyrrolidin-1-yl)pyrido[3,4-d]pyrimidin-4-amine (5a)

To a stirred solution of (R)-2-chloro-N-(1-(3-nitro-5-(trifluoromethyl)phenyl)ethyl)-6-(pyrrolidin-1-yl)pyrido[3,4-d]pyrimidin-4-amine (150 mg, 0.32 mmol) in IPA (5 mL) were added diisopropylethylamine (0.15 mL, 1.28 mmol) and morpholine (0.060 mL, 0.64 mmol) under nitrogen atmosphere then the reaction mixture was stirred at 120° C. for 16 h. Reaction progress was monitored by TLC. After completion of the reaction IPA was distilled off under reduced pressure to get crude compound which was treated with water and extracted with DCM (3×20 ml). The combined organic layers were washed with brine, dried over Na₂SO₄, concentrated and the crude obtained was purified by flash column chromatography using 2-3% MeOH in DCM as an eluent to afford the title compound as a yellow solid. (Yield: 90 mg, 54%); LCMS: m/z 518.2 [M+H]⁺.

### Step—b: Synthesis of (R)—N-(1-(3-amino-5-(trifluoromethyl)phenyl)ethyl)-2-morpholino-6-(pyrrolidin-1-yl)pyrido[3,4-d]pyrimidin-4-amine (5)

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 30%; ¹H-NMR (400 MHz, DMSO-d₆): δ 8.3 (s, 1H), 8.29 (d, J=6.3 Hz, 1H), 7.01 (s, 1H), 6.83 (s, 1H), 6.78 (s, 1H), 6.75 (s, 1H), 5.52 (bs, 2H), 5.25 (t, J=6.8 Hz, 1H), 3.56-3.52 (m, 8H), 3.40 (bs, 4H), 1.98 (bs, 4H), 1.56 (d, J=7.2 Hz, 3H); LCMS: m/z 488.20 [M+H]⁺.

### Example—6: (R)—N-(1-(3-amino-5-(trifluoromethyl)phenyl)ethyl)-2-(4-methylpiperazin-1-yl)-6-(pyrrolidin-1-yl)pyrido[3,4-d]pyrimidin-4-amine

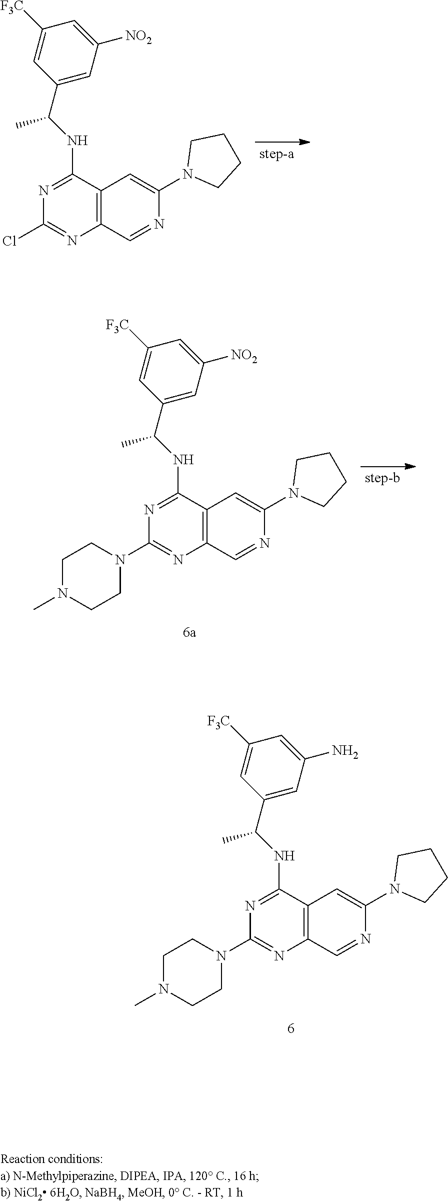 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(N2CCN(C)CC2)nc2cnc(N3CCCC3)cc12)c1cc([N+](=O)[O-])cc(C(F)(F)F)c1 ; C[C@@H](Nc1nc(N2CCN(C)CC2)nc2cnc(N3CCCC3)cc12)c1cc(N)cc(C(F)(F)F)c1]

### Step—a: Synthesis of (R)-2-(4-methylpiperazin-1-yl)-N-(1-(3-nitro-5-(trifluoromethyl)phenyl)ethyl)-6-(pyrrolidin-1-yl)pyrido[3,4-d]pyrimidin-4-amine (6a)

The title compound was synthesized using the same procedure which was followed for (R)-2-morpholino-N-(1-(3-nitro-5-(trifluoromethyl)phenyl)ethyl)-6-(pyrrolidin-1-yl)pyrido[3,4-d]pyrimidin-4-amine (5a) using (R)-2-chloro-N-(1-(3-nitro-5-(trifluoromethyl)phenyl)ethyl)-6-(pyrrolidin-1-yl)pyrido[3,4-d]pyrimidin-4-amine and N-methylpiperazine as starting materials (Yield: 72%). ¹H-NMR (400 MHz, DMSO-d₆): δ 8.56 (s, 1H), 8.46 (d, J=6.4 Hz, 1H), 8.36 (s, 1H), 8.35 (s, 1H), 8.34 (s, 1H), 6.95 (s, 1H), 5.50 (t, J=5.5 Hz, 1H), 3.57 (bs, 4H), 3.45 (d, J=5.3 Hz, 4H), 2.33 (bs, 4H), 2.19 (s, 3H), 1.95 (d, J=2.0 Hz, 4H), 1.66 (d, J=5.5 Hz, 3H); LCMS: m/z 531.30 [M+H]⁺.

### Step—b: Synthesis of (R)—N-(1-(3-amino-5-(trifluoromethyl)phenyl)ethyl)-2-(4-methylpiperazin-1-yl)-6-(pyrrolidin-1-yl)pyrido[3,4-d]pyrimidin-4-amine

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 71%; ¹H-NMR (400 MHz, DMSO-d₆): δ 8.35 (s, 1H), 8.26 (s, 1H), 7.0 (s, 1H), 6.83 (s, 1H), 6.78 (s, 1H), 6.67 (s, 1H), 5.52 (bs, 2H), 5.21 (d, J=7.5 Hz, 1H), 3.57 (bs, 4H), 3.3 (bs, 4H), 2.30 (bs, 4H), 2.18 (s, 3H), 1.98 (bs, 4H), 1.55 (d, J=7 Hz, 3H); LCMS: m/z 501.3 [M+H]⁺.

### Example—7: (R)—N4-(1-(3-amino-5-(trifluoromethyl)phenyl)ethyl)-N2-methyl-6-(pyrrolidin-1-yl)pyrido[3,4-d]pyrimidine-2,4-diamine

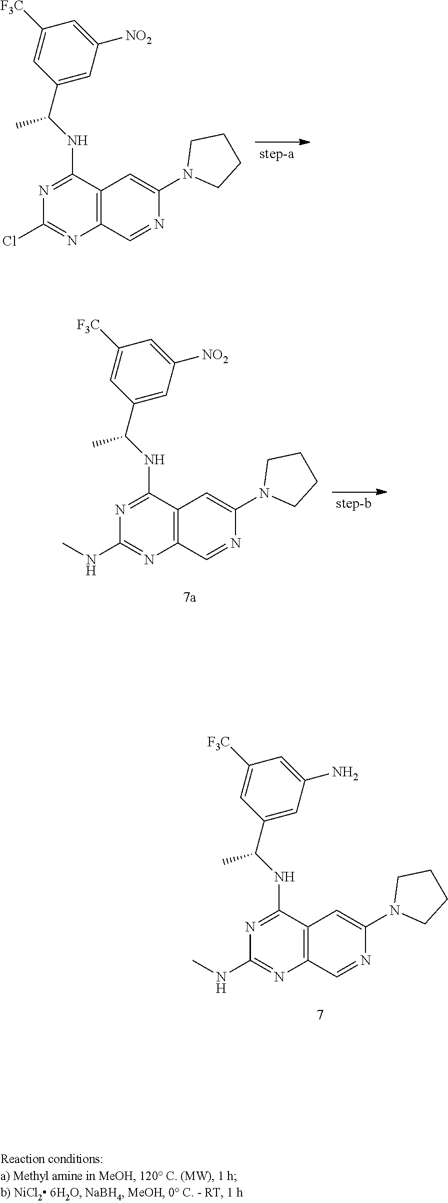 [SMILES: C[C@@H](Nc1nc(Cl)nc2cnc(N3CCCC3)cc12)c1cc([N+](=O)[O-])cc(C(F)(F)F)c1 ; CNc1nc(N[C@H](C)c2cc([N+](=O)[O-])cc(C(F)(F)F)c2)c2cc(N3CCCC3)ncc2n1 ; CNc1nc(N[C@H](C)c2cc(N)cc(C(F)(F)F)c2)c2cc(N3CCCC3)ncc2n1]

### Step—a: Synthesis of (R)—N2-methyl-N4-(1-(3-nitro-5-(trifluoromethyl)phenyl)ethyl)-6-(pyrrolidin-1-yl)pyrido[3,4-d]pyrimidine-2,4-diamine (7a)

25% methylamine in MeOH (2 mL) solution was added to (R)-2-chloro-N-(1-(3-nitro-5-(trifluoromethyl)phenyl)ethyl)-6-(pyrrolidin-1-yl)pyrido[3,4-d]pyrimidin-4-amine (80 mg, 0.17 mmol) in a microwave vial and irradiated at 120° C. for 1 h in Microwave reactor. Reaction mixture was quenched with water (10 mL), extracted with DCM (3×20 ml). The combined organic layers were washed with brine (20 mL), dried over Na₂SO₄, concentrated and the crude product obtained was purified by flash column chromatography using 2-3% MeOH in DCM as an eluent to afford the title compound as yellow a solid (Yield: 70 mg, 89%); ¹H-NMR (400 MHz, DMSO-d₆): δ 8.57 (s, 1H), 8.36 (s, 2H), 7.75 (s, 1H), 7.75 (bs, 1H), 6.98 (s, 1H), 6.37 (bs, 1H), 5.60 (t, J=7.1 Hz, 1H), 3.44 (d, J=3.4 Hz, 4H), 2.09 (s, 3H), 1.99 (bs, 4H), 1.67 (d, J=7 Hz, 3H); LCMS: m/z 462.20 [M+H]⁺.

### Step—b: Synthesis of (R)—N4-(1-(3-amino-5-(trifluoromethyl)phenyl)ethyl)-N2-methyl-6-(pyrrolidin-1-yl)pyrido[3,4-d]pyrimidine-2,4-diamine

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 25%; ¹H-NMR (400 MHz, DMSO-d₆): δ 8.36 (s, 1H), 8.10 (bs,1H), 7.01 (s, 1H), 6.82 (s, 2H), 6.69 (s, 1H), 6.23 (bs, 1H), 5.50 (bs, 2H), 5.46 (t, J=5.5 Hz, 1H), 3.40 (t, J=3.4 Hz, 4H), 2.75 (d, J=2.75 Hz, 3H), 1.98 (t, J=2.0 Hz, 4H), 1.55 (d, J=7.3 Hz, 3H); LCMS: m/z 432.20 [M+H]⁺.

### Example—8: (R)—N4-(1-(3-amino-5-(trifluoromethyl)phenyl)ethyl)-N2,N2-dimethyl-6-morpholinopyrido[3,4-d]pyrimidine-2,4-diamine

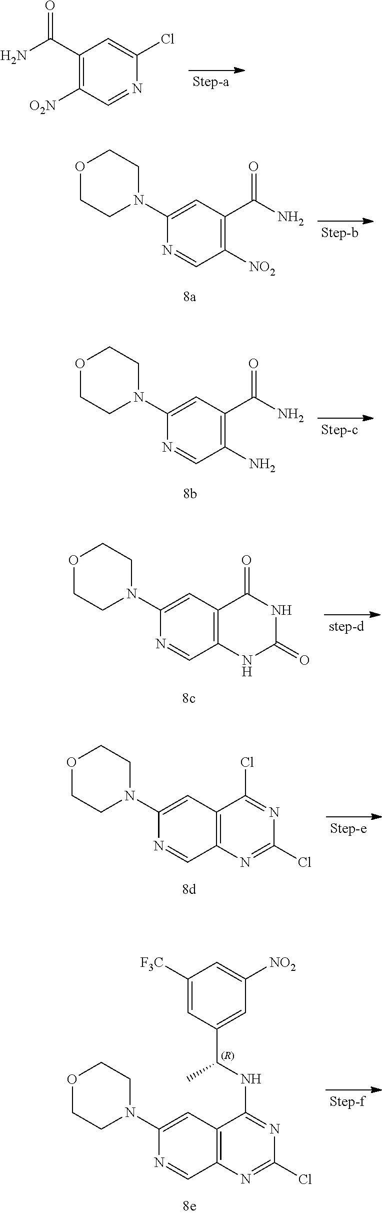

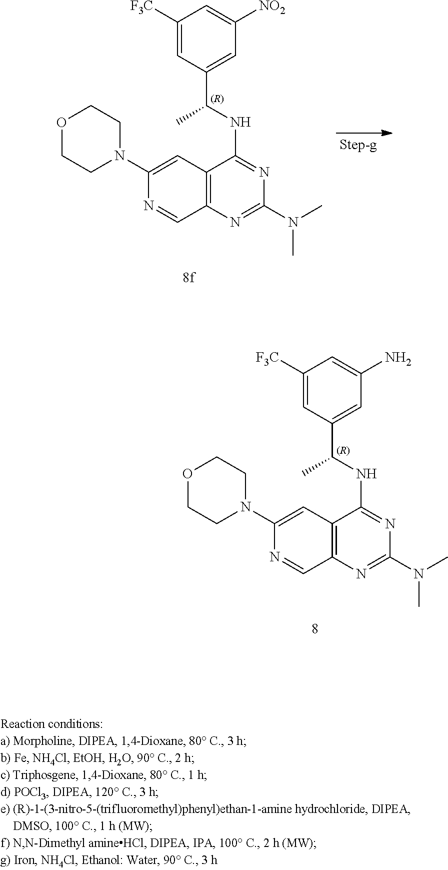

### Step—a: 2-morpholino-5-nitroisonicotinamide (8a)

The title compound was synthesized using the same procedure which was followed for 5-nitro-2-(pyrrolidin-1-yl)isonicotinamide (1b) using 2-chloro-5-nitroisonicotinamide and morpholine as starting materials and stirred at 80° C. for 3 h (Yield: Quantitative) ¹H NMR (400 MHz, DMSO-d₆) δ 9.37 (bs, 2H), 8.88 (s, 1H), 6.85 (s, 1H), 3.68 (s, 4H), 3.06 (s, 4H); LCMS: m/z 253.0 [M+H]⁺.

### Step—b: 5-amino-2-morpholinoisonicotinamide (8b)

To a solution of 2-morpholino-5-nitroisonicotinamide (1.5 g, 5.95 mmol) in ethanol (15 mL) and water (5 mL) was added iron powder (1.66 g, 26.76 mmol) and ammonium chloride (3.18 g, 59.5 mmol). The reaction mixture was heated at 90° C. for 2 h. Reaction progress was monitored by TLC. After completion of reaction, reaction mixture was filtered through celite bed, washed with methanol and filtrate was evaporated under reduce pressure. The residue was neutralized with saturated sodium bicarbonate solution (20 mL) and extracted with ethyl acetate (40 mL). The organic layer was washed with water (10 mL), brine solution (10 mL), dried over anhydrous sodium sulfate and concentrated under reduced pressure to afford 5-amino-2-morpholinoisonicotinamide as a brown semi solid. Yield: 0.9 g (68%); LCMS: m/z 223.1 [M+H]⁺.

### Step—c: 6-morpholinopyrido[3,4-d]pyrimidine-2,4(1H,3H)-dione (8c)

The title compound was synthesized using the same procedure which was followed for intermediate 6-(Pyrrolidin-1-yl)pyrido[3,4-d]pyrimidine-2,4(1H,3H)-dione (ld) (Yield: 85%); LCMS: m/z 249.1 [M+H]⁺.

### Step—d: 4-(2,4-dichloropyrido[3,4-d]pyrimidin-6-yl)morpholine (8 d)

The title compound was synthesized using the same procedure which was followed for intermediate 2,4-Dichloro-6-(pyrrolidin-1-yl)pyrido[3,4-d]pyrimidine (le). Yield: 33%; LCMS: m/z 285.0 [M+H]⁺.

### Step—e: Synthesis of (R)-2-chloro-6-morpholino-N-(1-(3-nitro-5-(trifluoromethyl)phenyl)ethyl)pyrido[3,4-d]pyrimidin-4-amine (8e)

To a stirred solution of 4-(2,4-dichloropyrido[3,4-d]pyrimidin-6-yl)morpholine (170 mg, 0.59 mmol) and (R)-1-(3-nitro-5-(trifluoromethyl)phenyl)ethan-1-amine hydrochloride (193 mg, 0.71 mmol) in DMSO (1.0 mL) in microwave vial were added DIPEA (0.036 mL, 0.21 mmol) at RT and irradiated in microwave reactor at 100° C. for 1 h. Reaction mixture was diluted with water (5 mL). Precipitated solid was filtered and dried under vacuum to afford the title compound as a pale yellow solid (yield: 190 mg, 66%); ¹H NMR (400 MHz, CDCl₃) δ 8.85 (s, 1H), 8.65 (s, 1H), 8.38 (s, 1H), 8.19 (s, 1H), 7.52 (s, 1H), 6.99 (s, 1H), 5.72-5.68 (m, 1H), 3.86 (bs, 4H), 3.61 (m, 4H), 1.90 (d, J=6.8 Hz, 3H); LCMS: m/z 483.1 [M+H]⁺.

### Step—f: (R)—N2,N2-dimethyl-6-morpholino-N4-(1-(3-nitro-5-(trifluoromethyl)phenyl)ethyl)pyrido[3,4-d]pyrimidine-2,4-diamine (8f)

The title compound was synthesized by using the same procedure which was followed for (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a) using (R)-2-chloro-6-morpholino-N-(1-(3-nitro-5-(trifluoromethyl)phenyl)ethyl)pyrido[3,4-d]pyrimidin-4-amine and N,N-dimethylamine hydrochloride as starting materials and irradiated in microwave reactor at 100° C. for 2 h (yield: 50%); LCMS: m/z 492.4 [M+H]⁺.

### Step—g: (R)—N4-(1-(3-amino-5-(trifluoromethyl)phenyl)ethyl)-N2,N2-dimethyl-6-morpholinopyrido[3,4-d]pyrimidine-2,4-diamine

To a stirred solution of (R)—N2,N2-dimethyl-6-morpholino-N4-(1-(3-nitro-5-(trifluoromethyl)phenyl)ethyl)pyrido[3,4-d]pyrimidine-2,4-diamine (65 mg, 0.13 mmol) in ethanol (3 mL) and water (1 mL) at ambient temperature was added ammonium chloride (70 mg, 1.32 mmol) and iron powder. The reaction mixture was heated at 90° C. for 3 h. Then it was cooled to RT, diluted with ethyl acetate (20 mL), filtered through celite bed. The organic layer was washed saturated NaHCO₃ (20 mL) solution, water (20 mL), brine (20 mL), dried over Na₂SO₄ and concentrated under reduced pressure. The residue was purified by combi flash column chromatography using 9-10% MeOH in DCM as an eluent to afford the title compound as a yellow solid (Yield: 15 mg, 25%). ¹H NMR (400 MHz, DMSO-d₆) δ 8.41 (s, 1H), 7.38 (s, 1H), 6.84 (s, 1H), 6.80 (s, 1H), 6.68 (s, 1H), 5.53 (s, 2H), 5.28 (t, J=6.8 Hz, 1H), 3.77 (t, J=4.4 Hz, 4H), 3.40 (t, J=4.8 Hz, 4H), 3.02 (bs, 6H), 1.90 (s, 1H), 1.55 (d, J=7.2 Hz, 3H); LCMS: m/z 462.3 [M+H]⁺.

### Example—9: (R)—N4-(1-(3-amino-5-(trifluoromethyl)phenyl)ethyl)-N2-(2-(dimethylamino)ethyl)-6-(pyrrolidin-1-yl)pyrido[3,4-d]pyrimidine-2,4-diamine

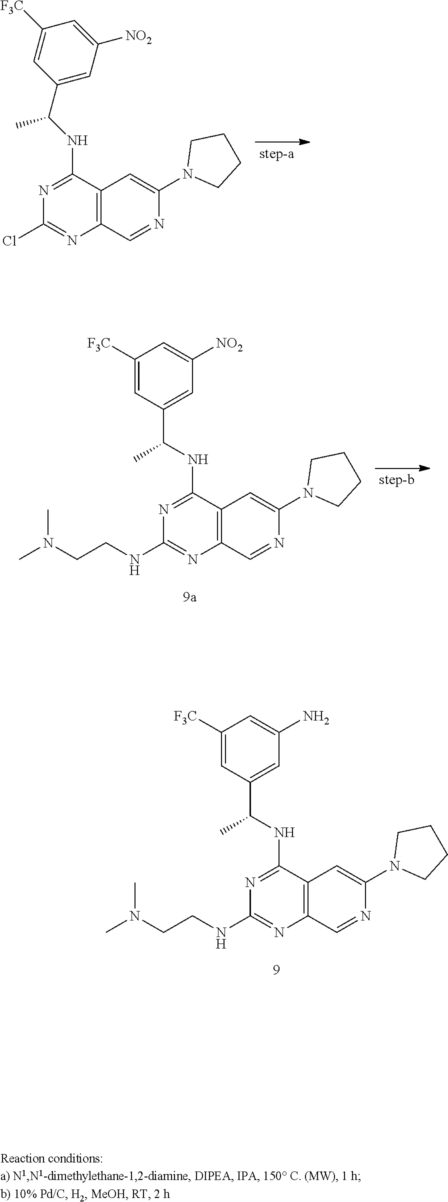 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(NCCN(C)C)nc2cnc(N3CCCC3)cc12)c1cc([N+](=O)[O-])cc(C(F)(F)F)c1 ; C[C@@H](Nc1nc(NCCN(C)C)nc2cnc(N3CCCC3)cc12)c1cc(N)cc(C(F)(F)F)c1]

### Step—a: (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a)

To a stirred solution of (R)-2-chloro-N-(1-(3-nitro-5-(trifluoromethyl)phenyl)ethyl)-6-(pyrrolidin-1-yl)pyrido[3,4-d]pyrimidin-4-amine (100 mg, 0.21 mmol) in IPA (1.5 mL) in microwave vial were added diisopropylethylamine (0.50 mL) and N1,N1-dimethylethane-1,2-diamine (1 mL) at RT and irradiated at 150° C. for 1 h in microwave reactor. Reaction mixture was quenched with water, extracted with DCM (3×20 ml). The combined organic layers were washed with brine, dried over Na₂SO₄, concentrated and the crude product was purified by combi flash column chromatography using 2-3% MeOH in DCM as an eluent to afford the title compound as a brown solid (yield: 80 mg, 74%); LCMS: m/z 519.20 [M+H]⁺.

### Step—b: (R)—N4-(1-(3-amino-5-(trifluoromethyl)phenyl)ethyl)-N2-(2-(dimethylamino)ethyl)-6-(pyrrolidin-1-yl)pyrido[3,4-d]pyrimidine-2,4-diamine

To a stirred solution of (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (50 mg, 0.096 mmol) in methanol (10 mL) was added slurry of 10% Pd—C(50% wet, 25 mg) in methanol (2 mL) and the reaction mixture was stirred under hydrogen atmosphere for 2 h. Reaction progress was monitored by TLC. The reaction mixture was filtered through celite bed, washed with methanol (10 mL) and evaporated under reduced pressure to afford the crude compound and which was purified by prep-HPLC (Yield: 20 mg, 42%); ¹H-NMR (400 MHz, DMSO-d₆): δ 8.32 (s, 1H), 8.12 (d, J=7.9 Hz, 1H), 6.98 (s, 1H), 6.82 (s, 1H), 6.80 (s, 1H), 6.90 (s, 1H), 5.87 (s, 1H), 5.54 (bs, 2H), 5.40 (t, J=7.1 Hz, 1H), 3.41 (d, J=3.4 Hz, 4H), 2.12 (s, 6H), 1.99 (d, J=6.4 Hz, 4H), 1.88 (s, 3H), 1.55 (d, J=7.3 Hz, 3H); LCMS: m/z 489.30 [M+H]⁺.

### Example—10: (R)—N4-(1-(3-amino-5-(trifluoromethyl)phenyl)ethyl)-N2-(2-(dimethylamino)ethyl)-N2-methyl-6-(pyrrolidin-1-yl)pyrido[3,4-d]pyrimidine-2,4-diamine

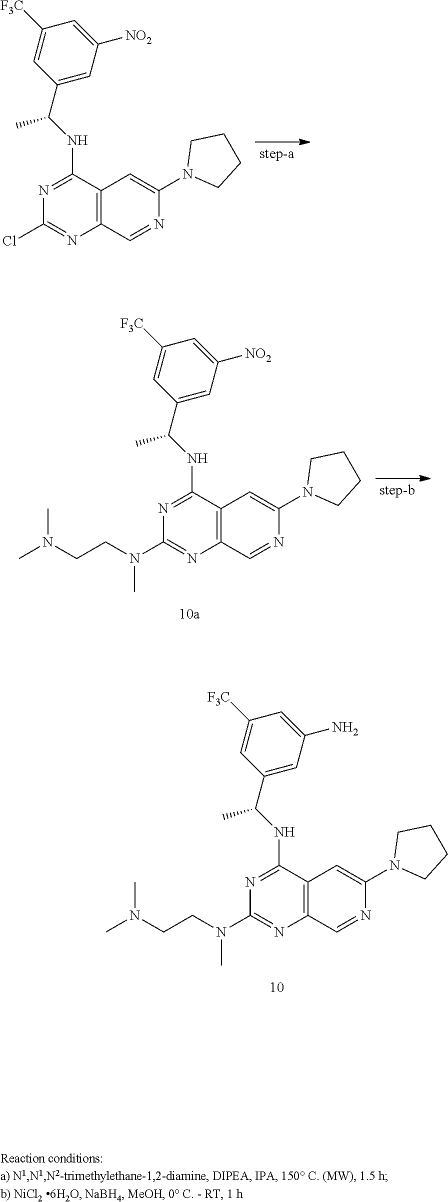 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(N(C)CCN(C)C)nc2cnc(N3CCCC3)cc12)c1cc([N+](=O)[O-])cc(C(F)(F)F)c1 ; C[C@@H](Nc1nc(N(C)CCN(C)C)nc2cnc(N3CCCC3)cc12)c1cc(N)cc(C(F)(F)F)c1]

### Step—a: (R)—N2-(2-(dimethylamino)ethyl)-N2-methyl-N4-(1-(3-nitro-5-(trifluoromethyl)phenyl)ethyl)-6-(pyrrolidin-1-yl)pyrido[3,4-d]pyrimidine-2,4-diamine (10a)

The title compound was synthesized by using the same procedure which was followed for (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a) using (R)-2-chloro-N-(1-(3-nitro-5-(trifluoromethyl)phenyl)ethyl)-6-(pyrrolidin-1-yl)pyrido[3,4-d]pyrimidin-4-amine and N1,N1,N2-trimethylethane-1,2-diamine as starting materials (Yield: 90%); LCMS: m/z 533.30 [M+H]⁺.

### Step—b: (R)—N4-(1-(3-amino-5-(trifluoromethyl)phenyl)ethyl)-N2-(2-(dimethylamino)ethyl)-N2-methyl-6-(pyrrolidin-1-yl)pyrido[3,4-d]pyrimidine-2,4-diamine

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1). Yield: 28%; ¹H-NMR (400 MHz, DMSO-d₆): δ8.34 (s, 1H), 8.16 (d, J=6.8 Hz, 1H), 7.0 (s,1H), 6.80 (s, 1H), 6.77 (s, 1H), 6.67 (s, 1H), 5.51 (bs, 2H), 5.32 (t, J=7.1 Hz, 1H), 3.58-3.50 (m, 2H), 3.42 (d, J=2.7 Hz, 4H), 3.0 (s, 3H), 2.27 (bs, 2H), 2.08 (s, 6H), 1.98 (t, J=6.3 Hz, 4H), 1.56 (d, J=6.9 Hz, 3H); LCMS: m/z 503.30 [M+H]⁺.

### Example—11: (R)-2-((4-((1-(3-amino-5-(trifluoromethyl)phenyl)ethyl)amino)-6-(pyrrolidin-1-yl)pyrido[3,4-d]pyrimidin-2-yl)oxy)ethan-1-ol

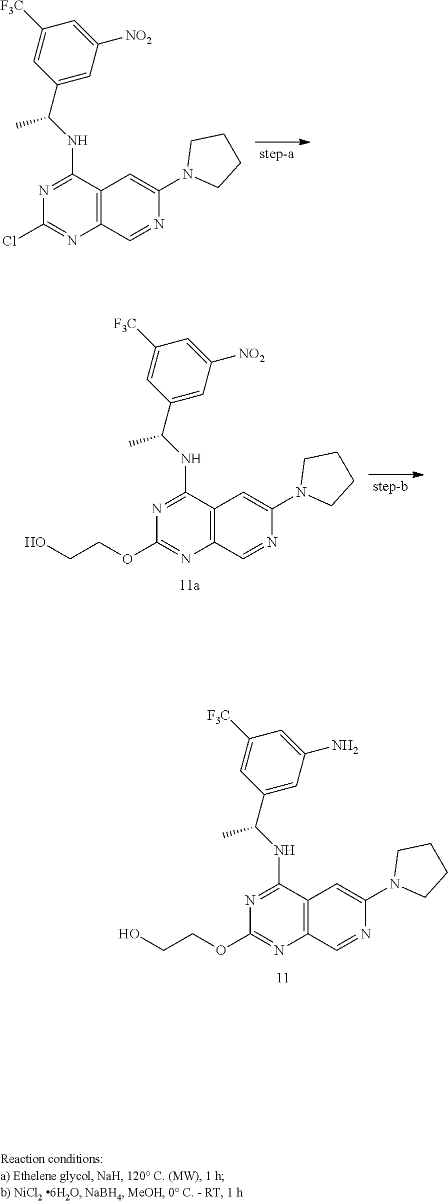 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(OCCO)nc2cnc(N3CCCC3)cc12)c1cc([N+](=O)[O-])cc(C(F)(F)F)c1 ; C[C@@H](Nc1nc(OCCO)nc2cnc(N3CCCC3)cc12)c1cc(N)cc(C(F)(F)F)c1]

### Step—a: (R)-2-((4-((1-(3-nitro-5-(trifluoromethyl)phenyl)ethyl)amino)-6-(pyrrolidin-1-yl)pyrido[3,4-d]pyrimidin-2-yl)oxy)ethan-1-ol (11a)

The title compound was synthesized by using the same procedure which was followed for (R)-2-(2-methoxyethoxy)-N-(1-(3-nitro-5-(trifluoromethyl)phenyl)ethyl)-6-(pyrrolidin-1-yl)pyrido[3,4-d]pyrimidin-4-amine (4a) using (R)-2-chloro-N-(1-(3-nitro-5-(trifluoromethyl)phenyl)ethyl)-6-(pyrrolidin-1-yl)pyrido[3,4-d]pyrimidin-4-amine and ethylene glycol as starting materials and heated at 120° C. for 1 h (Yield: 25%); LCMS: m/z 493.30 [M+H]⁺.

### Step—b: (R)-2-((4-((1-(3-amino-5-(trifluoromethyl)phenyl)ethyl)amino)-6-(pyrrolidin-1-yl)pyrido[3,4-d]pyrimidin-2-yl)oxy)ethan-1-ol

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1). Yield: 18%; ¹H-NMR (400 MHz, DMSO-d₆): δ8.51 (s, 1H), 8.48 (d, J=7.9 Hz, 1H), 7.10 (s, 1H), 6.83 (s, 1H), 6.80 (s, 1H), 6.70 (s, 1H), 5.57 (bs, 2H), 5.45 (t, J=7 Hz, 1H), 4.80 (t, J=5.7 Hz, 1H), 4.20 (t, J=5.2 Hz, 2H), 3.64 (q, J=5.2 Hz, 2H), 3.45 (d, J=3.3 Hz, 4H), 1.99 (bs, 4H), 1.55 (d, J=7 Hz, 3H); LCMS: m/z 463.30 [M+H]⁺.

### Example—12: (R)-2-((4-((1-(3-amino-5-(trifluoromethyl)phenyl)ethyl)amino)-6-(pyrrolidin-1-yl)pyrido[3,4-d]pyrimidin-2-yl)amino)ethan-1-ol

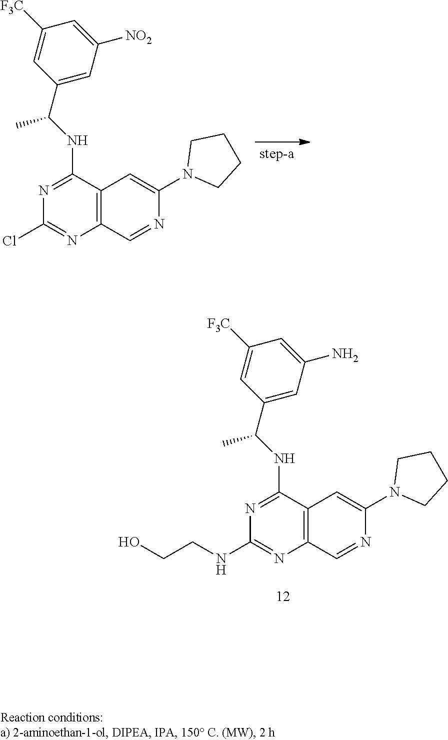 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(NCCO)nc2cnc(N3CCCC3)cc12)c1cc(N)cc(C(F)(F)F)c1]

To a solution of (R)-2-chloro-N-(1-(3-nitro-5-(trifluoromethyl)phenyl)ethyl)-6-(pyrrolidin-1-yl)pyrido[3,4-d]pyrimidin-4-amine (130 mg, 0.27 mmol) in isopropyl alcohol (7 mL) was added 2-aminoethan-1-ol (51 mg, 0.83 mmol) and DIPEA (0.15 mL, 0.83 mmol), in microwave vial and irradiated in microwave reactor for 2 h at 150° C. Once TLC analysis indicated completion of reaction, the reaction mixture was diluted with ethyl acetate (30 mL) and washed with water (20 mL) and brine (20 mL), dried over sodium sulfate, concentrated under vacuum to provide crude compound which was purified by prep-HPLC to obtain 18 mg (6%) of title product as yellow solid. ¹H NMR (400 MHz, DMSO-d₆) δ 8.31 (s, 1H), 8.13 (s, 1H), 6.98 (s, 1H), 6.82 (s, 2H), 6.69 (s, 1H), 6.06 (t, J=6.0 Hz, 1H), 5.54 (bs, 2H), 5.44 (bs, 1H), 3.47-3.40 (m, 8H), 1.97 (bs, 4H), 1.54 (d, J=7.2 Hz, 3H), One exchangeable proton not appeared; LCMS: m/z 462.2 [M+H]⁺.

### Example—13: (R)-2-((4-((1-(3-amino-5-(trifluoromethyl)phenyl)ethyl)amino)-6-(pyrrolidin-1-yl)pyrido[3,4-d]pyrimidin-2-yl)(methyl)amino)ethan-1-ol

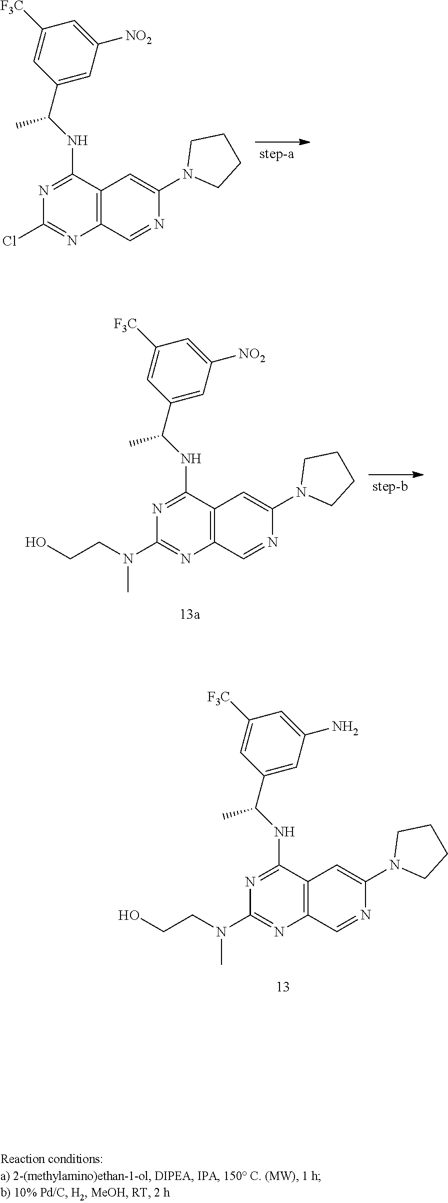 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(N(C)CCO)nc2cnc(N3CCCC3)cc12)c1cc([N+](=O)[O-])cc(C(F)(F)F)c1 ; C[C@@H](Nc1nc(N(C)CCO)nc2cnc(N3CCCC3)cc12)c1cc(N)cc(C(F)(F)F)c1]

### Step—a: (R)-2-(methyl(4-((1-(3-nitro-5-(trifluoromethyl)phenyl)ethyl)amino)-6-(pyrrolidin-1-yl)pyrido[3,4-d]pyrimidin-2-yl)amino)ethan-1-ol (13a)

The title compound was synthesized using the same procedure which was followed for (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a) using (R)-2-chloro-N-(1-(3-nitro-5-(trifluoromethyl)phenyl)ethyl)-6-(pyrrolidin-1-yl)pyrido[3,4-d]pyrimidin-4-amine and 2-(methylamino)ethan-1-ol as starting materials (yield: 57%); LCMS: m/z 506.20 [M+H]⁺.

### Step—b: (R)-2-((4-((1-(3-amino-5-(trifluoromethyl)phenyl)ethyl)amino)-6-(pyrrolidin-1-yl)pyrido[3,4-d]pyrimidin-2-yl)(methyl)amino)ethan-1-ol

The title compound was synthesized using the same procedure which was followed for (R)—N4-(1-(3-amino-5-(trifluoromethyl)phenyl)ethyl)-N2-(2-(dimethylamino)ethyl)-6-(pyrrolidin-1-yl)pyrido[3,4-d]pyrimidine-2,4-diamine (example-9). Yield: 42%; ¹H-NMR (400 MHz, DMSO-d₆): δ 8.33 (s, 1H), 8.17 (d, J=7.2 Hz, 1H), 6.99 (s, 1H), 6.83 (s, 1H), 6.79 (s, 1H), 6.67 (s, 1H), 5.51 (bs, 2H), 5.25 (t, J=7.1 Hz, 1H), 4.6 (bs, 1H), 3.51-3.47 (m, 4H), 3.41 (d, J=3.1, 4H), 3.04 (s, 3H), 1.99 (d, J=6.6 Hz, 4H), 1.55 (d, J=7.0, 3H) LCMS: m/z 476.20 [M+H]⁺.

### Example—14: (R)—N4-(1-(3-amino-5-(trifluoromethyl)phenyl)ethyl)-N2-(2-methoxyethyl)-6-(pyrrolidin-1-yl)pyrido[3,4-d]pyrimidine-2,4-diamine

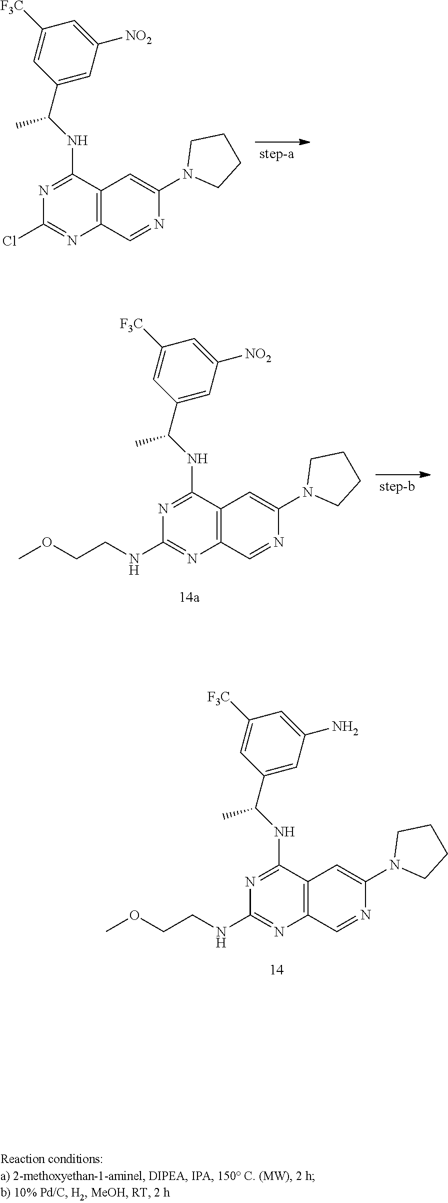 [SMILES: C[C@@H](Nc1nc(Cl)nc2cnc(N3CCCC3)cc12)c1cc([N+](=O)[O-])cc(C(F)(F)F)c1 ; COCCNc1nc(N[C@H](C)c2cc([N+](=O)[O-])cc(C(F)(F)F)c2)c2cc(N3CCCC3)ncc2n1 ; COCCNc1nc(N[C@H](C)c2cc(N)cc(C(F)(F)F)c2)c2cc(N3CCCC3)ncc2n1]

### Step—a: (R)—N2-(2-methoxyethyl)-N4-(1-(3-nitro-5-(trifluoromethyl)phenyl)ethyl)-6-(pyrrolidin-1-yl)pyrido[3,4-d]pyrimidine-2,4-diamine (14a)

The title compound was synthesized using the same procedure which was followed for (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a) using (R)-2-chloro-N-(1-(3-nitro-5-(trifluoromethyl)phenyl)ethyl)-6-(pyrrolidin-1-yl)pyrido[3,4-d]pyrimidin-4-amine and 2-methoxyethan-1-amine as starting materials (Yield: 40%); LCMS: m/z 506.20 [M+H]⁺.

### Step—b: (R)—N4-(1-(3-amino-5-(trifluoromethyl)phenyl)ethyl)-N2-(2-methoxyethyl)-6-(pyrrolidin-1-yl)pyrido[3,4-d]pyrimidine-2,4-diamine

The title compound was synthesized using the same procedure which was followed for (R)—N4-(1-(3-amino-5-(trifluoromethyl)phenyl)ethyl)-N2-(2-(dimethylamino)ethyl)-6-(pyrrolidin-1-yl)pyrido[3,4-d]pyrimidine-2,4-diamine (example-9); Yield: 50%; ¹H-NMR (400 MHz, CD₃OD): δ8.32 (s, 1H), 8.12 (d, J=7.2 Hz, 1H), 6.98 (s, 1H), 6.81 (s, 1H), 6.69 (s, 1H), 6.07 (bs, 1H), 5.53 (s, 2H), 5.41 (bs, 1H), 6 3.41-3.39 (m, 8H), 1.98 (t, J=6.3 Hz, 4H), 1.54 (d, J=7 Hz, 3H); LCMS: m/z 476.20 [M+H]⁺.

### Example—15: (R)—N4-(1-(3-amino-5-(trifluoromethyl)phenyl)ethyl)-N2-(2-methoxyethyl)-N2-methyl-6-(pyrrolidin-1-yl)pyrido[3,4-d]pyrimidine-2,4-diamine

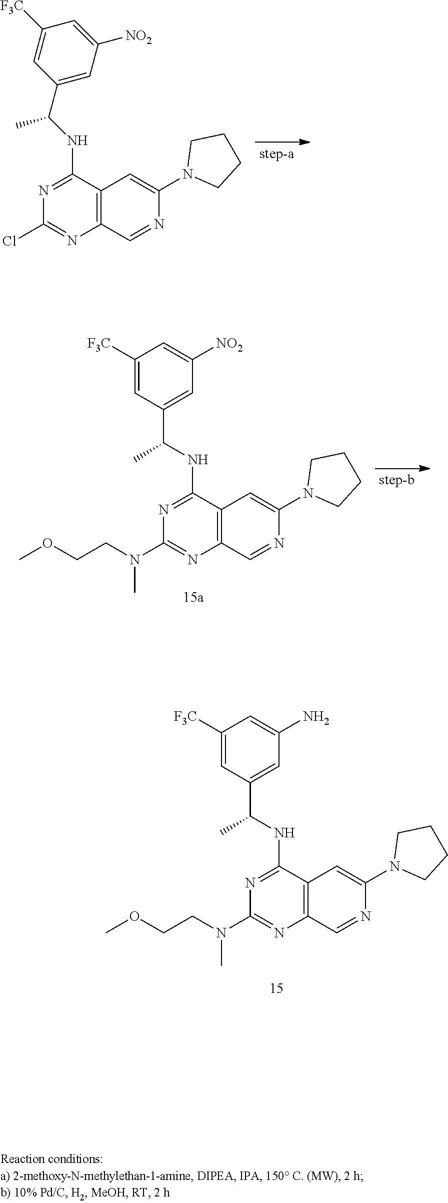 [SMILES: C[C@@H](Nc1nc(Cl)nc2cnc(N3CCCC3)cc12)c1cc([N+](=O)[O-])cc(C(F)(F)F)c1 ; COCCN(C)c1nc(N[C@H](C)c2cc([N+](=O)[O-])cc(C(F)(F)F)c2)c2cc(N3CCCC3)ncc2n1 ; COCCN(C)c1nc(N[C@H](C)c2cc(N)cc(C(F)(F)F)c2)c2cc(N3CCCC3)ncc2n1]

### Step—a: (R)—N2-(2-methoxyethyl)-N2-methyl-N4-(1-(3-nitro-5-(trifluoromethyl)phenyl)ethyl)-6-(pyrrolidin-1-yl)pyrido[3,4-d]pyrimidine-2,4-diamine (15a)

The title compound was synthesized using the same procedure which was followed for (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a) using (R)-2-chloro-N-(1-(3-nitro-5-(trifluoromethyl)phenyl)ethyl)-6-(pyrrolidin-1-yl)pyrido[3,4-d]pyrimidin-4-amine and 2-methoxy-N-methylethan-1-amine as starting materials (Yield: 81%); LCMS: m/z 520.30 [M+H]⁺.

### Step—b: (R)—N4-(1-(3-amino-5-(trifluoromethyl)phenyl)ethyl)-N2-(2-methoxyethyl)-N2-methyl-6-(pyrrolidin-1-yl)pyrido[3,4-d]pyrimidine-2,4-diamine

The title compound was synthesized using the same procedure which was followed for (R)—N4-(1-(3-amino-5-(trifluoromethyl)phenyl)ethyl)-N2-(2-(dimethylamino)ethyl)-6-(pyrrolidin-1-yl)pyrido[3,4-d]pyrimidine-2,4-diamine (example-9).Yield: 80%; ¹H-NMR (400 MHz, CD₃OD): δ8.34 (s, 1H), 6.91 (s, 1H), 6.80 (s, 1H), 6.78 (s, 1H), 6.68 (s, 1H), 5.17 (q, t, J=7.2 Hz, 1H), 3.71-3.66 (m, 1H), 3.42-3.32 (m, 5H), 3.30-3.24 (m, 2H), 3.12 (s, 3H), 3.0 (s, 3H), 1.95-190 (m, 4H), 1.52 (d, J=7.2 Hz, 3H); LCMS: m/z 490.20 [M+H]⁺.

### Example—16: (R)—N-(1-(3-amino-5-(trifluoromethyl)phenyl)ethyl)-2-(azetidin-1-yl)-6-(pyrrolidin-1-yl)pyrido[3,4-d]pyrimidin-4-amine

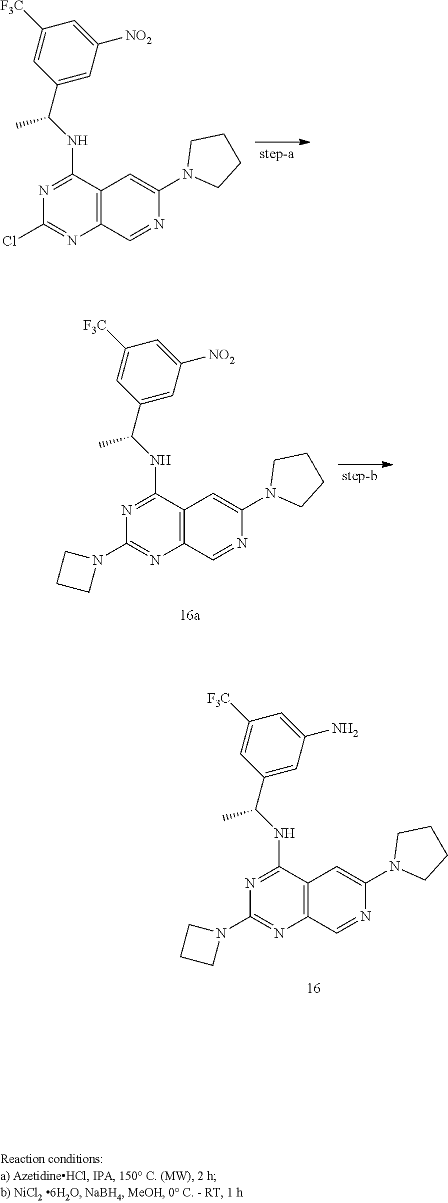 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(N2CCC2)nc2cnc(N3CCCC3)cc12)c1cc([N+](=O)[O-])cc(C(F)(F)F)c1 ; C[C@@H](Nc1nc(N2CCC2)nc2cnc(N3CCCC3)cc12)c1cc(N)cc(C(F)(F)F)c1]

### Step—a: (R)-2-(azetidin-1-yl)-N-(1-(3-nitro-5-(trifluoromethyl)phenyl)ethyl)-6-(pyrrolidin-1-yl)pyrido[3,4-d]pyrimidin-4-amine (16a)

The title compound was synthesized using the same procedure which was followed for (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a) using (R)-2-chloro-N-(1-(3-nitro-5-(trifluoromethyl)phenyl)ethyl)-6-(pyrrolidin-1-yl)pyrido[3,4-d]pyrimidin-4-amine and Azetidine hydrochloride as starting materials (Yield: 42%); LCMS: m/z 488.20 [M+H]⁺.

### Step—b: (R)—N-(1-(3-amino-5-(trifluoromethyl)phenyl)ethyl)-2-(azetidin-1-yl)-6-(pyrrolidin-1-yl)pyrido[3,4-d]pyrimidin-4-amine

The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1). Yield: 30%; ¹H-NMR (400 MHz, DMSO-d₆) δ 8.41 (s, 1H), 7.07 (s, 1H), 6.84 (s, 1H), 6.80 (s, 1H), 6.70 (s, 1H), 5.56 (bs, 2H), 5.32 (t, J=7.1 Hz, 1H), 4.04 (bs, 2H), 3.94 (bs, 2H), 3.43 (d, J=2.4 Hz, 4H), 2.25 (bs, 2H), 2.04 (d, J=6.4 Hz, 4H), 1.57 (d, J=7 Hz, 3H), One exchangeable proton not observed in NMR; LCMS: m/z 458.20 [M+H]⁺.

### Example—17: (R)—N-(1-(3-amino-5-(trifluoromethyl)phenyl)ethyl)-2,6-di(pyrrolidin-1-yl)pyrido[3,4-d]pyrimidin-4-amine

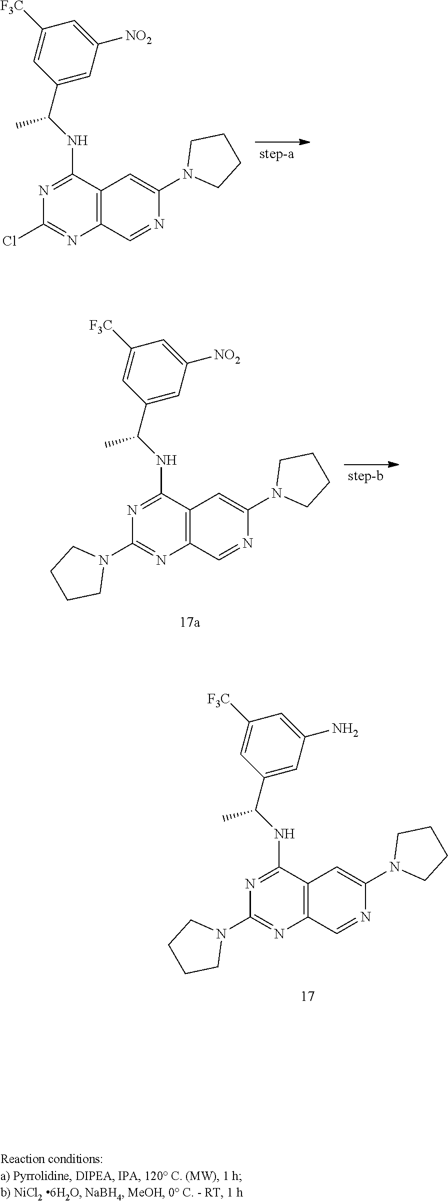 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(N2CCCC2)nc2cnc(N3CCCC3)cc12)c1cc([N+](=O)[O-])cc(C(F)(F)F)c1 ; C[C@@H](Nc1nc(N2CCCC2)nc2cnc(N3CCCC3)cc12)c1cc(N)cc(C(F)(F)F)c1]

### Step—a: (R)—N-(1-(3-nitro-5-(trifluoromethyl)phenyl)ethyl)-2,6-di(pyrrolidin-1-yl)pyrido[3,4-d]pyrimidin-4-amine (17a)

The title compound was synthesized using the same procedure which was followed for (R)—N2-(2-(dimethylamino)ethyl)-N4-(1-(3-nitro-5-(trifluoromethyl)phenyl)ethyl)-6-(pyrrolidin-1-yl)pyrido[3,4-d]pyrimidine-2,4-diamine (9a) using (R)-2-chloro-N-(1-(3-nitro-5-(trifluoromethyl)phenyl)ethyl)-6-(pyrrolidin-1-yl)pyrido[3,4-d]pyrimidin-4-amine and pyrrolidine as starting materials and heated at 120° C. for 1 h (Yield: 70%); LCMS: m/z 502.20 [M+H]⁺.

Step—b: (R)—N-(1-(3-amino-5-(trifluoromethyl)phenyl)ethyl)-2,6-di(pyrrolidin-1-yl)pyrido[3,4-d]pyrimidin-4-amine The title compound was synthesized using the same procedure which was followed for (R)—N-(1-(3-amino-5-(trifluoromethyl)phenyl)ethyl)-2-chloro-6-(pyrrolidin-1-yl)pyrido[3,4d]pyrimidin-4-amine (example-1). Yield: 50%; ¹H-NMR (400 MHz, CD₃OD): δ8.37 (s, 1H), 6.98 (s, 1H), 6.85 (s, 1H), 6.82 (s, 1H), 6.70 (s, 1H), 5.27 (q, J=7.1 Hz, 1H), 3.47 (bs, 2H), 3.41-3.35 (m, 6H), 1.99 (t, J=6.6 Hz, 4H), 1.88 (bs, 4H), 1.57 (d, J=7 Hz, 3H); LCMS: m/z 472.20 [M+H]⁺.

### Example—18: (R)—N4-(1-(3-amino-5-(trifluoromethyl)phenyl)ethyl)-N2-methyl-6-morpholinopyrido[3,4-d]pyrimidine-2,4-diamine

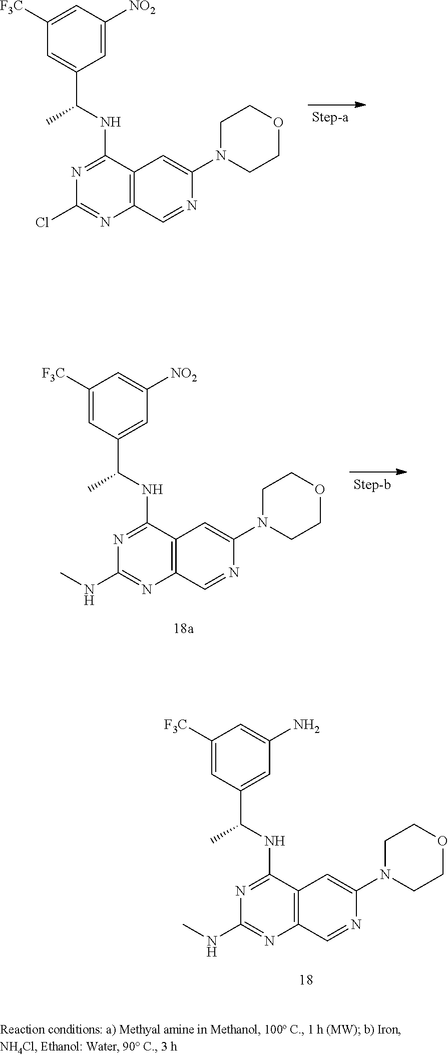 [SMILES: C[C@@H](Nc1nc(Cl)nc2cnc(N3CCOCC3)cc12)c1cc([N+](=O)[O-])cc(C(F)(F)F)c1 ; CNc1nc(N[C@H](C)c2cc([N+](=O)[O-])cc(C(F)(F)F)c2)c2cc(N3CCOCC3)ncc2n1 ; CNc1nc(N[C@H](C)c2cc(N)cc(C(F)(F)F)c2)c2cc(N3CCOCC3)ncc2n1]

### Step—a: Synthesis of (R)—N2-methyl-6-morpholino-N4-(1-(3-nitro-5-(trifluoromethyl)phenyl)ethyl)pyrido[3,4-d]pyrimidine-2,4-diamine (18a)

The title compound was synthesized using the same procedure which was followed for (R)—N2-methyl-N4-(1-(3-nitro-5-(trifluoromethyl)phenyl)ethyl)-6-(pyrrolidin-1-yl)pyrido[3,4-d]pyrimidine-2,4-diamine (7a) using (R)-2-chloro-6-morpholino-N-(1-(3-nitro-5-(trifluoromethyl)phenyl)ethyl)pyrido[3,4-d]pyrimidin-4-amine and methyl amine in methanol as starting materials and irradiated in microwave reactor at 100° C. (Yield: 95%). LCMS: m/z 478.2 [M+H]⁺.

### Step—b: Synthesis of (R)—N4-(1-(3-amino-5-(trifluoromethyl)phenyl)ethyl)-N2-methyl-6-morpholinopyrido[3,4-d]pyrimidine-2,4-diamine

The title compound was synthesized using the same procedure which was followed for (R)—N4-(1-(3-amino-5-(trifluoromethyl)phenyl)ethyl)-N2,N2-dimethyl-6-morpholinopyrido[3,4-d]pyrimidine-2,4-diamine (example-8); Yield: 43%. ¹H NMR (400 MHz, DMSO-d₆) δ 8.41 (s, 1H), 7.31 (s, 1H), 6.81 (bs, 2H), 6.70 (s, 1H), 5.56 (s, 2H), 5.50-5.40 (m, 1H), 3.77-3.75 (m, 4H), 3.41-3.39 (m, 4H), 2.77 (d, J=4.8 Hz, 3H), 1.55 (d, J=7.2 Hz, 3H), Two exchangeable protons not observed in NMR; LCMS: m/z 448.2 [M+H]⁺.

TABLE 1
Compounds in table-1 were prepared by using process analogous to the
preparation of example-1

| | | | 1H NMR (400 MHz, DMSO- |
|---|---|---|---|
| Example | | | d₆): δ ppm/LC-MS: |
| # | Structure | IUPAC Name | m/z |
| 19 | 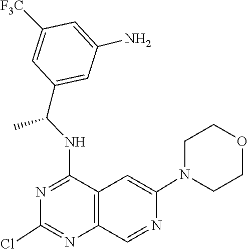 [SMILES: C[C@@H](Nc1nc(Cl)nc2cnc(N3CCOCC3)cc12)c1cc(N)cc(C(F)(F)F)c1] | (R)-N-(1-(3-amino- 5- (trifluoromethyl)phe- nyl)ethyl)-2-chloro- 6- morpholinopyrido[3,4- d]pyrimidin-4- amine | 8.91 (d, J = 8.0 Hz, 1H), 8.70 (s, 1H), 7.47 (s, 1H), 6.85 (s, 1H), 6.81 (s, 1H), 6.73 (s, 1H), 5.60 (bs, 2H), 5.44 (quin, J = 7.2 Hz, 1H), 3.78 (t, J = 4.4 Hz, 4H), 3.54 (t, J = 4.4 Hz, 4H), 1.57 (d, J = 6.8 Hz, 3H). 453.10 [M + H]⁺ |
| 20 | 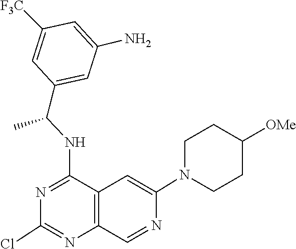 [SMILES: COC1CCN(c2cc3c(N[C@H](C)c4cc(N)cc(C(F)(F)F)c4)nc(Cl)nc3cn2)CC1] | (R)-N-(1-(3-amino- 5- (trifluoromethyl)phe- nyl)ethyl)-2-chloro- 6-(4- methoxypiperidin-1- yl)pyrido[3,4- d]pyrimidin-4-amine | 8.88 (d, J = 8.4 Hz, 1H), 8.66 (s, 1H), 7.48 (s, 1H), 6.85 (s, 1H), 6.80 (s, 1H), 6.73 (s, 1H), 5.61 (bs, 2H), 5.43 (quin, J = 7.2 Hz, 1H), 5.04- 5.02 (m, 2H), 3.32 (1H merged with DMSO moisture peak), 3.30 (s, 3H), 3.28- 3.24 (m, 2H), 1.95 (bs, 2 H), 1.57 (d, J = 7.2 Hz, 3H), 1.49- 1.48 (m, 2H) 481.10 [M + H]⁺ |
| 21 | 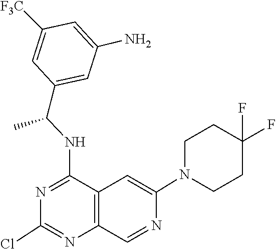 [SMILES: C[C@@H](Nc1nc(Cl)nc2cnc(N3CCC(F)(F)CC3)cc12)c1cc(N)cc(C(F)(F)F)c1] | (R)-N-(1-(3-amino- 5- (trifluoromethyl)phe- nyl)ethyl)-2-chloro- 6-(4,4- difluoropiperidin-1- yl)pyrido[3,4- d]pyrimidin-4-amine | 8.91 (d, J = 7.6 Hz, 1H), 8.70 (s, 1H), δ 7.59 (s, 1H), 6.85 (s, 1H), 6.81 (s, 1H), 6.73 (s, 1H), 5.61 (bs, 2H), 5.44 (quin, J = 7.2 Hz, 1H), 3.80 (t, J = 5.6 Hz, 4H), 2.11- 2.04 (m, 4H), 1.58 (d, J = 7.2 Hz, 3H) 487.30 [M + H]⁺. |
| 22 | 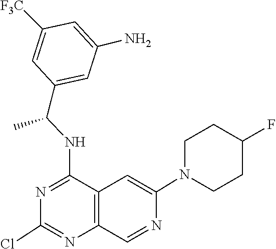 [SMILES: C[C@@H](Nc1nc(Cl)nc2cnc(N3CCC(F)CC3)cc12)c1cc(N)cc(C(F)(F)F)c1] | (R)-N-(1-(3-amino- 5- (trifluoromethyl)phe- nyl)ethyl)-2-chloro- 6-(4-fluoropiperidin- 1-yl)pyrido[3,4- d]pyrimidin-4-amine | 8.88 (d, J = 7.6 Hz, 1H), 8.67 (s, 1H), 7.52 (s, 1H), 6.85 (s, 1H), 6.81 (s, 1H), 6.73 (s, 1H), 5.60 (bs, 2H), 5.44 (quin, J = 7.2 Hz, 1H), 5.00- 4.86 (m, 1H), 3.82-3.80 (m, 2H), 3.63-3.58 (m, 2H), 2.02-1.95 (m, 2H), 1.82- 1.72 (m, 2H), 1.58 (d, J = 7.2 Hz, 3H) 469.20 [M + H]⁺. |
| 23 | 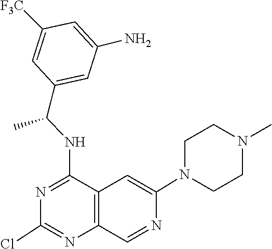 [SMILES: C[C@@H](Nc1nc(Cl)nc2cnc(N3CCN(C)CC3)cc12)c1cc(N)cc(C(F)(F)F)c1] | (R)-N-(1-(3-amino- 5- (trifluoromethyl)phe- nyl)ethyl)-2-chloro- 6-(4- methylpiperazin-1- yl)pyrido[3,4- d]pyrimidin-4-amine | 8.89 (d, J = 8.0 Hz, 1H), 8.67 (s, 1H), δ 7.47 (s, 1H), 6.85 (s, 1H), 6.81 (s, 1H), 6.73 (s, 1H), 5.60 (bs, 2H), 5.43 (quin, J = 7.2 Hz, 1H), 3.58 (bs, 4H), 2.50 (4H merged with DMSO peak), 2.33 (s, 3H), 1.57 (d, J = 6.8 Hz, 3H) 466.10 [M + H]⁺ |
| 24 | 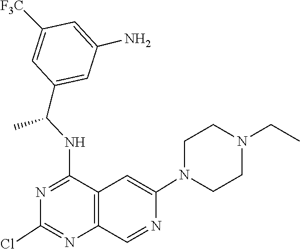 [SMILES: CCN1CCN(c2cc3c(N[C@H](C)c4cc(N)cc(C(F)(F)F)c4)nc(Cl)nc3cn2)CC1] | (R)-N-(1-(3-amino- 5- (trifluoromethyl)phe- nyl)ethyl)-2-chloro- 6-(4-ethylpiperazin- 1-yl)pyrido[3,4- d]pyrimidin-4-amine | 8.90 (d, J = 7.6 Hz, 1H), 8.67 (s, 1H), δ 7.47 (s, 1H), 6.85 (s, 1H), 6.80 (s, 1H), 6.73 (s, 1H), 5.60 (bs, 2H), 5.44 (quin, J = 7.2 Hz, 1H), 3.58 (t, J = 4.4 Hz, 4H), 2.50 (4H merged with DMSO peak), 2.39 (q, J = 7.2 Hz, 2H), 1.57 (d, J = 6.8 Hz, 3H), 1.05 (t, J = 7.2 Hz, 3H) 480.20 [M + H]⁺ |
| 25 | 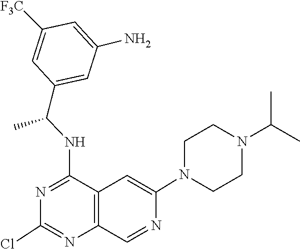 [SMILES: CC(C)N1CCN(c2cc3c(N[C@H](C)c4cc(N)cc(C(F)(F)F)c4)nc(Cl)nc3cn2)CC1] | (R)-N-(1-(3-amino- 5- (trifluoromethyl)phe- nyl)ethyl)-2-chloro- 6-(4- isopropylpiperazin- 1-yl)pyrido[3,4- d]pyrimidin-4-amine | 8.90 (d, J = 7.6 Hz, 1H), 8.67 (s, 1H), 7.46 (s, 1H), 6.85 (s, 1H), 6.80 (s, 1H), 6.73 (s, 1H), 5.60 (bs, 2H), 5.43 (quin, J = 7.2 Hz, 1H), 3.57 (bs, 4H), 2.73-2.70 (m, 1H), 2.59 (t, J = 4.8 Hz, 4H), 1.57 (d, J = 7.2 Hz, 3H), 1.01 (d, J = 6.4 Hz, 6H) 494.30 [M + H]⁺ |
| 26 | 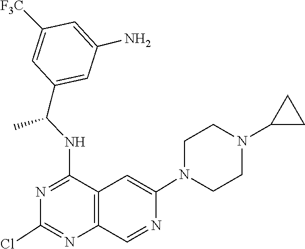 [SMILES: C[C@@H](Nc1nc(Cl)nc2cnc(N3CCN(C4CC4)CC3)cc12)c1cc(N)cc(C(F)(F)F)c1] | (R)-N-(1-(3-amino- 5- (trifluoromethyl)phe- nyl)ethyl)-2-chloro- 6-(4- cyclopropylpiperazin- 1-yl)pyrido[3,4- d]pyrimidin-4-amine | 8.90 (d, J = 7.6 Hz, 1H), 8.67 (s, 1H), 7.47 (s, 1H), 6.85 (s, 1H), 6.81 (s, 1H), 6.73 (s, 1H), 5.60 (bs, 2H), 5.43 (quin, J = 7.2 Hz, 1H), 3.55 (bs, 4H), 2.69 (bs, 4H), 1.67 (bs, 1H), 1.57 (d, J = 7.2 Hz, 3H), 0.46 (d, J = 6.0 Hz, 2H), 0.39 (bs, 2H) 492.10 [M + H]⁺. |
| 27 | 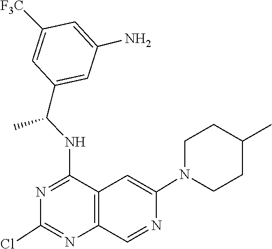 [SMILES: CC1CCN(c2cc3c(N[C@H](C)c4cc(N)cc(C(F)(F)F)c4)nc(Cl)nc3cn2)CC1] | (R)-N-(1-(3-amino- 5- (trifluoromethyl)phe- nyl)ethyl)-2-chloro- 6-(4- methylpiperidin-1- yl)pyrido[3,4- d]pyrimidin-4-amine | 8.87 (d, J = 8.0 Hz, 1H), 8.65 (s, 1H), δ 7.44 (s, 1H), 6.85 (s, 1H), 6.80 (s, 1H), 6.73 (s, 1H), 5.61 (bs, 2H), 5.43 (quin, J = 7.6 Hz, 1H), 4.39- 4.37 (m, 2H), 2.87 (t, J = 12.4 Hz, 2H), 1.73 (d, J = 12.8 Hz, 2H), 1.70-1.64 (m, 1H), 1.57 (d, J = 6.8 Hz, 3H), 1.27- 1.14 (m, 2H), 0.95 (d, J = 6.4 Hz, 3H) 465.20 [M + H]⁺ |
| 28 | 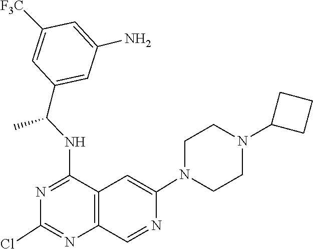 [SMILES: C[C@@H](Nc1nc(Cl)nc2cnc(N3CCN(C4CCC4)CC3)cc12)c1cc(N)cc(C(F)(F)F)c1] | (R)-N-(1-(3-amino- 5- (trifluoromethyl)phe- nyl)ethyl)-2-chloro- 6-(4- cyclobutylpiperazin- 1-yl)pyrido[3,4- d]pyrimidin-4-amine | 8.90 (d, J = 7.6 Hz, 1H), 8.67 (s, 1H), 7.47 (s, 1H), 6.85 (s, 1H), 6.80 (s, 1H), 6.73 (s, 1H), 5.60 (bs, 2H), 5.43 (quin, J = 7.2 Hz, 1H), 3.57 (bs, 4H), 2.77-2.73 (m, 1H), 2.39 (bs, 4H), 2.04-1.96 (m, 2H), 1.86-1.82 (m, 2H), 1.70-1.60 (m, 2H), 1.57 (d, J = 6.8 Hz, 3H) 506.10 [M + H]⁺ |
| 29 | 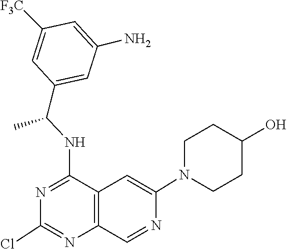 [SMILES: C[C@@H](Nc1nc(Cl)nc2cnc(N3CCC(O)CC3)cc12)c1cc(N)cc(C(F)(F)F)c1] | (R)-1-(4-((1-(3- amino-5- (trifluoromethyl)phe- nyl)ethyl)amino)-2- chloropyrido[3,4- d]pyrimidin-6- yl)piperidin-4-ol | 8.86 (d, J = 8.4 Hz, 1H), 8.65 (s, 1H), 7.46 (s, 1H), 6.85 (s, 1H), 6.81 (s, 1H), 6.73 (s, 1H), 5.60 (s, 2H), 5.43 (quin, J = 7.4 Hz, 1H), 4.75 (d, J = 4.0 Hz, 1H), 4.12-4.02 (m, 2H), 3.78-3.70 (m, 1H), 3.27-3.17 (m, 2H), 1.88- 1.83 (m, 2H), 1.57 (d, J = 6.8 Hz, 3H), 1.46-1.40 (m, 2H) 467.10 [M + H]⁺ |

TABLE 2
Compounds in table-2 were prepared by using process analogous to the
preparation of example-7

| | | | 1H NMR (400 MHz, |
|---|---|---|---|
| | | | DMSO-d₆): δ ppm/LC- |
| Example # | Structure | IUPAC Name | MS: m/z |
| 30 | 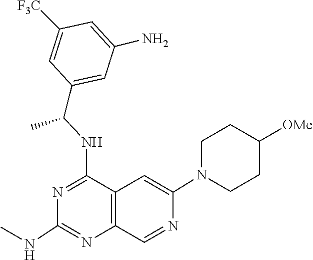 [SMILES: CNc1nc(N[C@H](C)c2cc(N)cc(C(F)(F)F)c2)c2cc(N3CCC(OC)CC3)ncc2n1] | (R)-N4-(1-(3-amino- 5- (trifluoromethyl)phenyl) ethyl)-6-(4- methoxypiperidin-1- yl)-N2- methylpyrido[3,4- d]pyrimidine-2,4- diamine | 8.37 (s, 1H), 8.13 (d, J = 7.6 Hz, 1H), 7.35 (s, 1H), 6.82 (bs, 2H), 6.70 (s, 1H), 6.24 (q, J = 4.4 Hz, 1H), 5.53 (s, 2H), 5.43 (quin, J = 7.2 Hz, 1H), 3.92 (bs, 2H), 3.52 (1H merged with DMSO moisture peak), 3.31 (s, 3H), 3.10-3.04 (m, 2H), 2.74 (d, J = 4.4 Hz, 3H), 1.97-1.95 (m, 2H), 1.54 (d, J = 7.2 Hz, 3H), 1.50-1.47 (m, 2H) 476.10 [M + H]⁺ |
| 31 | 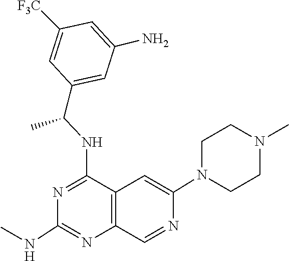 | (R)-N4-(1-(3-amino- 5- (trifluoromethyl)phenyl) ethyl)-N2-methyl- 6-(4-methylpiperazin- 1-yl)pyrido[3,4- d]pyrimidine-2,4- diamine | 8.37 (s, 1H), 8.10 (bs, 1H), 7.33 (s, 1H), 6.81 (s, 2H), 6.69 (s, 1H), 6.25 (q, J = 4.8 Hz, 1H), 5.54 (s, 2H), 5.44- 5.41 (m, 1H), 3.43 (bs, 4H), 2.74 (d, J = 4.8 Hz, 3H), 2.50 (4H, merged with DMSO peak), 2.24 (s, 3H), 1.54 (d, J = 7.2 Hz, 3H) 461.20 [M + H]⁺ |
| 32 | 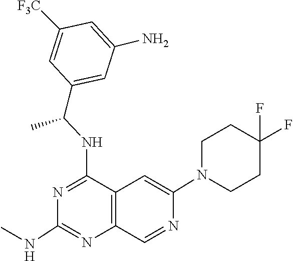 [SMILES: CNc1nc(N[C@H](C)c2cc(N)cc(C(F)(F)F)c2)c2cc(N3CCC(F)(F)CC3)ncc2n1] | (R)-N4-(1-(3-amino- 5- (trifluoromethyl)phenyl) ethyl)-6-(4,4- difluoropiperidin-1- yl)-N2- methylpyrido[3,4- d]pyrimidine-2,4- diamine | 8.39 (s, 1H), 8.10 (bs, 1H), 7.45 (s, 1H), 6.82 (s, 2H), 6.70 (s, 1H), 6.31 (q, J = 4.8 Hz, 1H), 5.54 (s, 2H), 5.50- 5.40 (m, 1H), 3.65 (t, J = 5.6 Hz, 4H), 2.74 (d, J = 4.8 Hz, 3H), 2.08-2.02 (m, 4H), 1.54 (d, J = 6.8 Hz, 3H) 482.20 [M + H]⁺ |
| 33 | 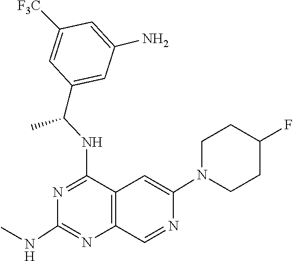 [SMILES: CNc1nc(N[C@H](C)c2cc(N)cc(C(F)(F)F)c2)c2cc(N3CCC(F)CC3)ncc2n1] | (R)-N4-(1-(3-amino- 5- (trifluoromethyl)phenyl) ethyl)-6-(4- fluoropiperidin-1-yl)- N2-methylpyrido[3,4- d]pyrimidine-2,4- diamine | 8.37 (s, 1H), 8.10 (bs, 1H), 7.39 (s, 1H), 6.82 (s, 2H), 6.70 (s, 1H), 6.26 (q, J = 4.8 Hz, 1H), 5.54 (s, 2H), 5.46- 5.42 (m, 1H), 4.95-4.83 (m, 1H), 3.71 (bs, 2H), 3.41- 3.38 (m, 2H), 2.74 (d, J = 4.8 Hz, 3H), 1.99 (bs, 2H), 1.78 (bs, 2H), 1.54 (d, J = 6.8 Hz, 3H) 464.30 [M + H]⁺ |

TABLE 3
Compounds in table-3 were prepared by using process analogous to the
preparation of example-9

| | | | 1H NMR (400 MHz, |
|---|---|---|---|
| | | | DMSO-d₆): δppm/LC- |
| Example # | Structure | IUPAC Name | MS: m/z |
| 34 | 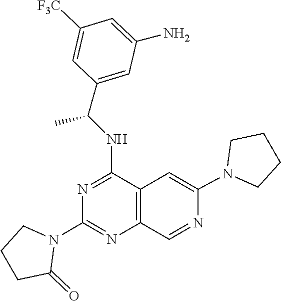 [SMILES: C[C@@H](Nc1nc(N2CCCC2=O)nc2cnc(N3CCCC3)cc12)c1cc(N)cc(C(F)(F)F)c1] | (R)-1-(4-((1-(3- amino-5- (trifluoromethyl) phenyl)ethyl)amino)- 6-(pyrrolidin-1- yl)pyrido[3,4- d]pyrimidin-2- yl)pyrrolidin-2-one | 8.58 (s, 1H), 8.49 (d, J = 7.2 Hz, 1H), 7.05 (s, 1H), 6.91 (s, 1H), 6.86 (s, 1H), 6.69 (s, 1H), 5.52 (s, 2H), 5.51- 5.40 (m, 1H), 3.94-3.88 (m, 1H), 3.80-3.74 (m, 1H), 3.50-3.45 (m, 4H), 2.50 (2H merged with DMSO peak), 2.02-1.98 (m, 4H), 1.95-1.90 (m, 2H), 1.57 (d, J = 7.2 Hz, 3H) 486.30 [M + H]⁺ |
| 35 | 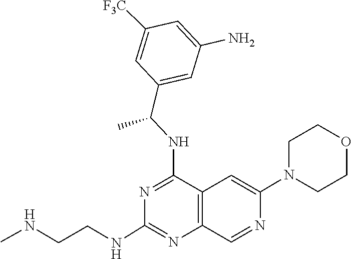 [SMILES: CNCCNc1nc(N[C@H](C)c2cc(N)cc(C(F)(F)F)c2)c2cc(N3CCOCC3)ncc2n1] | (R)-N4-(1-(3-amino- 5- (trifluoromethyl) phenyl)ethyl)-N2-(2- (methylamino)ethyl)- 6- morpholinopyrido [3,4-d]pyrimidine-2,4- diamine | 8.37 (s, 1H), 8.15 (bs, 1H), 7.35 (s, 1H), 6.81 (s, 2H), 6.70 (s, 1H), 6.25 (bs, 1H), 5.54 (bs, 2H), 5.43-5.38 (m, 1H), 3.77 (t, J = 4.8 Hz, 4H), 3.50-3.30 (m, 2H), 3.39 (t, J = 4.8 Hz, 4H), 3.04 (bs, 1H), 2.50 (2H merged with DMSO peak), 2.26 (s, 3H), 1.54 (d, J = 7.2 Hz, 3H) 491.40 [M + H]⁺ |
| 36 | 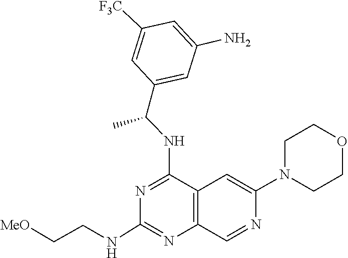 [SMILES: COCCNc1nc(N[C@H](C)c2cc(N)cc(C(F)(F)F)c2)c2cc(N3CCOCC3)ncc2n1] | (R)-N4-(1-(3-amino- 5- (trifluoromethyl) phenyl)ethyl)-N2-(2- methoxyethyl)-6- morpholinopyrido [3,4-d]pyrimidine-2,4- diamine | 8.39 (s, 1H), 7.39 (s, 1H), 6.81 (bs, 2H), 6.70 (s, 1H), 5.55 (bs, 2H), 5.44-5.38 (m, 1H), 3.77 (t, J = 4.8 Hz, 4H), 3.41-3.39 (m, 6H), 3.33 (2H merged with DMSO moisture peak), 3.20 (s, 3H), 1.55 (d, J = 6.8 Hz, 3H) (Two exchangeable protons not observed in NMR) 492.20 [M + H]⁺ |
| 37 | 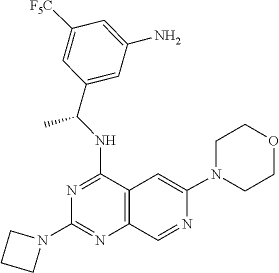 | (R)-N-(1-(3-amino-5- (trifluoromethyl) phenyl) ethyl)-2-(azetidin- 1-yl)-6- morpholinopyrido [3,4-d] pyrimidin-4-amine | 8.42 (s, 1H), 7.38 (s, 1H), 6.83 (s, 1H), 6.80 (s, 1H), 6.69 (s, 1H), 5.54 (bs, 2H), 5.27 (quin, J = 7.2 Hz, 1H), 4.0-3.85 (m, 2H), 3.39 (m, 2H), 3.77 (t, J = 4.8 Hz, 4H), 3.41 (bs, 4H), 2.23- 2.19 (m, 2H), 1.54 (d, J = 7.2 Hz, 3H) (One exchangeable proton not observed in NMR) 474.50 [M + H]⁺ |
| 38 | 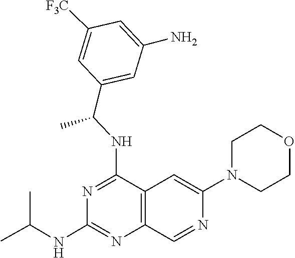 [SMILES: CC(C)Nc1nc(N[C@H](C)c2cc(N)cc(C(F)(F)F)c2)c2cc(N3CCOCC3)ncc2n1] | (R)-N4-(1-(3-amino- 5- (trifluoromethyl) phenyl)ethyl)-N2- isopropyl-6- morpholinopyrido [3,4-d]pyrimidine-2,4- diamine | (CD₃OD) 8.34 (s, 1H), 7.32 (s, 1H), 6.83 (s, 1H), 6.81 (s, 1H), 6.70 (s, 1H), 5.35- 5.30 (m, 1H), 4.01-3.95 (m, 1H), 3.75 (t, J = 4.8 Hz, 4H), 3.40 (t, J = 4.8 Hz, 4H), 1.55 (d, J = 6.8 Hz, 3H), 1.14 (d, J = 6.4 Hz, 3H), 0.96 (d, J = 6.0 Hz, 3H) 476.30 [M + H]⁺ |
| 39 | 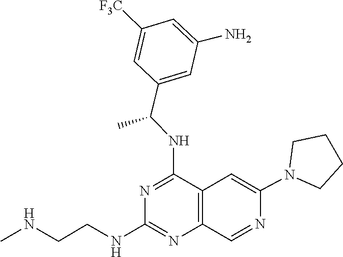 [SMILES: CNCCNc1nc(N[C@H](C)c2cc(N)cc(C(F)(F)F)c2)c2cc(N3CCCC3)ncc2n1] | (R)-N4-(1-(3-amino- 5- (trifluoromethyl) phenyl)ethyl)-N2-(2- (methylamino)ethyl)- 6-(pyrrolidin-1- yl)pyrido[3,4- d]pyrimidine-2,4- diamine | 8.33 (s, 1H), 8.11 (d, J = 8.0 Hz, 1H), 6.98 (s, 1H), 6.82 (bs, 2H), 6.69 (s, 1H), 6.07 (bs, 1H), 5.54 (s, 2H), 5.44- 5.40 (m, 1H), 3.46-3.40 (m, 4H), 3.32 (2H merged with DMSO moisture peak), 2.61-2.58 (m, 2H), 2.27 (s, 3H), 2.01-1.93 (m, 4H), 1.54 (d, J = 6.8 Hz, 3H) (One exchangeable proton not observed in NMR) 475.20 [M + H]⁺ |
| 40 | 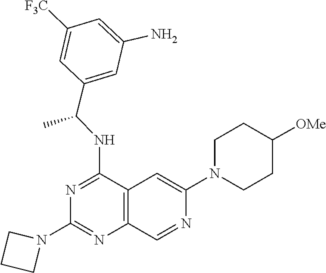 [SMILES: COC1CCN(c2cc3c(N[C@H](C)c4cc(N)cc(C(F)(F)F)c4)nc(N4CCC4)nc3cn2)CC1] | (R)-N-(1-(3-amino-5- (trifluoromethyl) phenyl) ethyl)-2-(azetidin- 1-yl)-6-(4- methoxypiperidin-1- yl)pyrido[3,4- d]pyrimidin-4-amine | 8.38 (s, 1H), 8.27 (d, J = 7.2 Hz, 1H), 7.35 (s, 1H), 6.83 (s, 1H), 6.80 (s, 1H), 6.68 (s, 1H), 5.53 (s, 2H), 5.28- 5.24 (m, 1H), 3.95-3.91 (m, 4H), 3.86-3.80 (m, 2H), 3.43-3.38 (m, 1H), 3.29 (s, 3H), 3.11-3.06 (m, 2H), 2.21-2.16 (m, 2H), 2.01-1.92 (m, 2H), 1.54 (d, J = 6.8 Hz, 3H), 1.50-1.47 (m, 2H) 502.30 [M + H]⁺ |
| 41 | 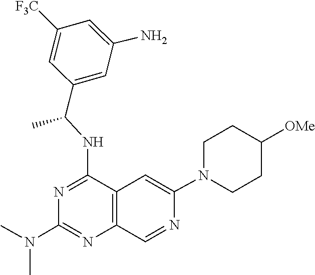 [SMILES: COC1CCN(c2cc3c(N[C@H](C)c4cc(N)cc(C(F)(F)F)c4)nc(N(C)C)nc3cn2)CC1] | (R)-N4-(1-(3-amino- 5- (trifluoromethyl) phenyl)ethyl)-6-(4- methoxypiperidin-1- yl)-N2,N2- dimethylpyrido[3,4- d]pyrimidine-2,4- diamine | 8.37 (s, 1H), 8.24 (bs, 1H), 7.37 (s, 1H), 6.84 (s, 1H), 6.79 (s, 1H), 6.68 (s, 1H), 5.53 (bs, 2H), 5.27 (quin, J = 6.8 Hz, 1H), 3.95-3.90 (m, 2H), 3.42-3.38 (m, 1H), 3.30 (s, 3H), 3.10- 3.05 (m, 2H), 3.0 (s, 6H), 1.98-1.95 (m, 2H), 1.55 (d, J = 6.8 Hz, 3H), 1.51-1.45 (m, 2H) 490.30 [M + H]⁺ |
| 42 | 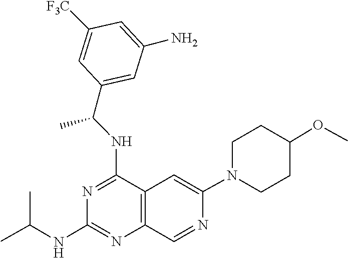 [SMILES: COC1CCN(c2cc3c(N[C@H](C)c4cc(N)cc(C(F)(F)F)c4)nc(NC(C)C)nc3cn2)CC1] | (R)-N4-(1-(3-amino- 5- (trifluoromethyl) phenyl)ethyl)-N2- isopropyl-6-(4- methoxypiperidin-1- yl)pyrido[3,4- d]pyrimidine-2,4- diamine | 8.34 (s, 1H), 8.20 (bs, 1H), 7.37 (s, 1H), 6.81 (s, 1H), 6.80 (s, 1H), 6.69 (s, 1H), 6.18 (bs, 1H), 5.54 (s, 2H), 5.50-5.45 (m, 1H), 4.0- 3.90 (m, 3H), 3.45-3.40 (m, 1H), 3.29 (s, 3H), 3.08 (t, J = 10.4 Hz, 2H), 1.97- 1.94 (m, 2H), 1.54 (d, J = 7.2 Hz, 3H), 1.50-1.48 (m, 2H), 1.11 (d, J = 6.0 Hz, 3H) 1.0 (bs, 3H) 504.20 [M + H]⁺ |
| 43 | 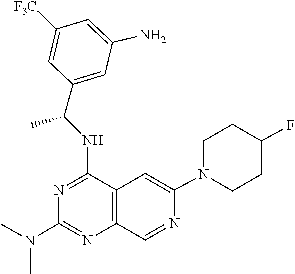 [SMILES: C[C@@H](Nc1nc(N(C)C)nc2cnc(N3CCC(F)CC3)cc12)c1cc(N)cc(C(F)(F)F)c1] | (R)-N4-(1-(3-amino- 5- (trifluoromethyl) phenyl)ethyl)-6-(4- fluoropiperidin-1-yl)- N2,N2- dimethylpyrido[3,4- d]pyrimidine-2,4- diamine | 8.39 (s, 1H), 8.25 (bs, 1H), 7.40 (s, 1H), 6.84 (s, 1H), 6.80 (s, 1H), 6.68 (s, 1H), 5.53 (bs, 2H), 5.28 (quin, J = 6.8 Hz, 1H), 4.86-4.83 (m, 1H), 3.76-3.68 (m, 4H), 3.00 (s, 6H), 2.05- 1.92 (m, 2H), 1.84-1.74 (m, 2H), 1.55 (d, J = 7.2 Hz, 3H) 478.20 [M + H]⁺ |
| 44 | 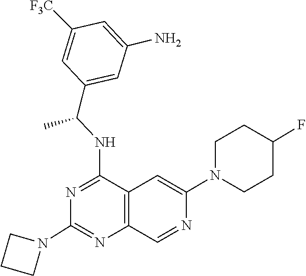 [SMILES: C[C@@H](Nc1nc(N2CCC2)nc2cnc(N3CCC(F)CC3)cc12)c1cc(N)cc(C(F)(F)F)c1] | (R)-N-(1-(3-amino-5- (trifluoromethyl) phenyl)ethyl)-2- (azetidin-1-yl)-6-(4- fluoropiperidin-1- yl)pyrido[3,4- d]pyrimidin-4-amine | 8.44 (s, 1H), 7.48 (bs, 1H), 6.84 (s, 1H), 6.80 (s, 1H), 6.70 (s, 1H), 5.56 (s, 2H), 5.29 (quin, J = 6.8 Hz, 1H), 5.0-4.92 (m, 1H), 4.88- 4.80 (m, 1H), 4.10-3.88 (m, 4H), 3.78-3.70 (m, 2H), 3.50-3.42 (m, 2H), 2.30-2.20 (m, 2H), 2.06- 1.92 (m, 2H), 1.84-1.72 (m, 2H), 1.56 (d, J = 7.2 Hz, 3H) 490.30 [M + H]⁺ |
| 45 | 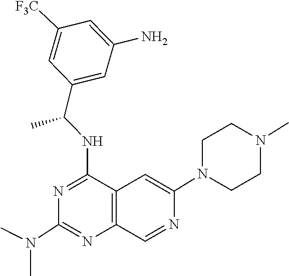 | (R)-N4-(1-(3-amino- 5- (trifluoromethyl) phenyl)ethyl)-N2,N2- dimethyl-6-(4- methylpiperazin-1- yl)pyrido[3,4- d]pyrimidine-2,4- diamine | 8.40 (s, 1H), 8.27 (bs, 1H), 7.41 (s, 1H), 6.85 (s, 1H), 6.80 (s, 1H), 6.69 (s, 1H), 5.54 (s, 2H), 5.28 (quin, J = 6.8 Hz, 1H), 3.33 (bs, 4H), 3.02 (s, 6H), 2.50 (7H merged with DMSO peak), 1.55 (d, J = 6.8 Hz, 3H) 475.10 [M + H]⁺ |
| 46 | 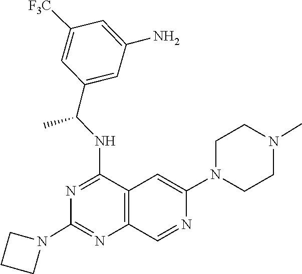 [SMILES: C[C@@H](Nc1nc(N2CCC2)nc2cnc(N3CCN(C)CC3)cc12)c1cc(N)cc(C(F)(F)F)c1] | (R)-N-(1-(3-amino-5- (trifluoromethyl) phenyl)ethyl)-2- (azetidin- 1-yl)-6-(4- methylpiperazin-1- yl)pyrido[3,4- d]pyrimidin-4-amine | 8.39 (s, 1H), 8.28 (d, J = 7.2 Hz, 1H), 7.34 (s, 1H), 6.83 (s, 1H), 6.80 (s, 1H), 6.68 (s, 1H), 5.53 (s, 2H), 5.26 (quin, J = 6.8 Hz, 1H), 3.94 (q, J = 8.0 Hz, 2H), 3.85 (q, J = 8.4 Hz, 2H), 3.44 (bs, 4H), 2.50 (4H merged with DMSO peak), 2.25 (s, 3H), 2.20-2.16 (m, 2H), 1.54 (d, J = 7.2 Hz, 3H) 487.30 [M + H]⁺ |
| 47 | 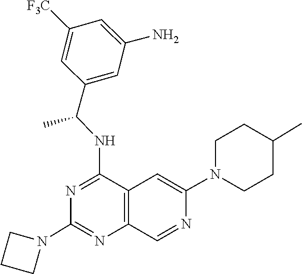 [SMILES: CC1CCN(c2cc3c(N[C@H](C)c4cc(N)cc(C(F)(F)F)c4)nc(N4CCC4)nc3cn2)CC1] | (R)-N-(1-(3-amino-5- (trifluoromethyl) phenyl)ethyl)-2- (azetidin- 1-yl)-6-(4- methylpiperidin-1- yl)pyrido[3,4- d]pyrimidin-4-amine | 8.39 (s, 1H), 7.35 (bs, 1H), 6.83 (s, 1H), 6.80 (s, 1H), 6.69 (s, 1H), 5.55 (bs, 2H), 5.28 (quin, J = 6.8 Hz, 1H), 4.24 (d, J = 12.0 Hz, 2H), 3.98 (bs, 2H), 3.88 (bs, 2H), 2.74 (t, J = 11.6 Hz, 2H), 2.24-2.18 (m, 2H), 1.73 (d, J = 12.0 Hz, 2H), 1.62- 1.59 (m, 1H), 1.55 (d, J = 7.2 Hz, 3H), 1.20-1.16 (m, 2H), 0.95 (d, J = 6.4 Hz, 3H) (One exchangeable proton not observed in NMR) 486.20 [M + H]⁺ |
| 48 | 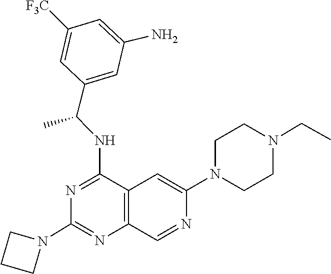 [SMILES: CCN1CCN(c2cc3c(N[C@H](C)c4cc(N)cc(C(F)(F)F)c4)nc(N4CCC4)nc3cn2)CC1] | (R)-N-(1-(3-amino-5- (trifluoromethyl) phenyl)ethyl)-2- (azetidin- 1-yl)-6-(4- ethylpiperazin-1- yl)pyrido[3,4- d]pyrimidin-4-amine | 8.39 (s, 1H), 8.29 (d, J = 6.0 Hz, 1H), 7.34 (s, 1H), 6.83 (s, 1H), 6.80 (s, 1H), 6.68 (s, 1H), 5.53 (s, 2H), 5.26 (quin, J = 6.8 Hz, 1H), 3.93 (q, J = 7.6 Hz, 2H), 3.84 (q, J = 7.6 Hz, 2H), 3.44 (bs, 4H), 3.0 (bs, 2H), 2.50 (4H merged with DMSO peak), 2.20-2.16 (m, 2H), 1.54 (d, J = 6.8 Hz, 3H), 1.06 (t, J = 7.2Hz, 3H) 501. 30 [M + H]⁺ |
| 49 | 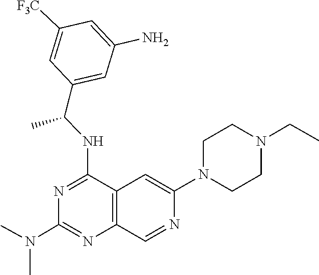 [SMILES: CCN1CCN(c2cc3c(N[C@H](C)c4cc(N)cc(C(F)(F)F)c4)nc(N(C)C)nc3cn2)CC1] | (R)-N4-(1-(3-amino- 5- (trifluoromethyl) phenyl)ethyl)-6-(4- ethylpiperazin-1-yl)- N2,N2- dimethylpyrido[3,4- d]pyrimidine-2,4- diamine | 8.37 (s, 1H), 8.24 (d, J = 6.8 Hz, 1H), 7.34 (s, 1H), 6.84 (s, 1H), 6.80 (s, 1H), 6.68 (s, 1H), 5.53 (s, 2H), 5.27 (quin, J = 7.2 Hz, 1H), 3.43 (bs, 4H), 3.0 (s, 6H), 2.50 (4H merged with DMSO peak), 2.39 (q, J = 7.2 Hz, 2H), 1.54 (d, J = 7.2 Hz, 3H), 1.06 (d, J = 7.2 Hz, 3H) 489.10 [M + H]⁺ |
| 50 | 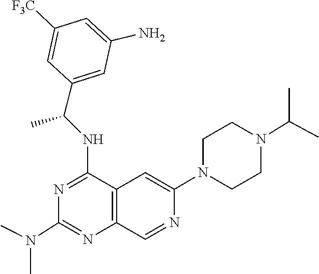 [SMILES: CC(C)N1CCN(c2cc3c(N[C@H](C)c4cc(N)cc(C(F)(F)F)c4)nc(N(C)C)nc3cn2)CC1] | (R)-N4-(1-(3-amino- 5- (trifluoromethyl) phenyl)ethyl)-6-(4- isopropylpiperazin-1- yl)-N2,N2- dimethylpyrido[3,4- d]pyrimidine-2,4- diamine | 8.37 (s, 1H), 8.25 (d, J = 6.8 Hz, 1H), 7.34 (s, 1H), 6.84 (s, 1H), 6.80 (s, 1H), 6.67 (s, 1H), 5.52 (s, 2H), 5.28 (quin, J = 6.8 Hz, 1H), 3.42 (bs, 4H), 3.0 (s, 6H), 2.73- 2.67 (m, 1H), 2.61 (bs, 4H), 1.56 (d, J = 8.0 Hz, 3H), 1.04 (d, J = 6.4 Hz, 6H) 503.20 [M + H]⁺ |
| 51 | 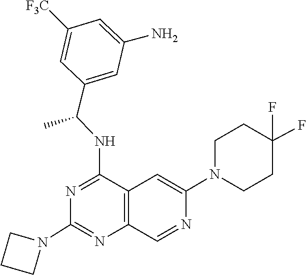 [SMILES: C[C@@H](Nc1nc(N2CCC2)nc2cnc(N3CCC(F)(F)CC3)cc12)c1cc(N)cc(C(F)(F)F)c1] | (R)-N-(1-(3-amino-5- (trifluoromethyl) phenyl)ethyl)-2- (azetidin- 1-yl)-6-(4,4- difluoropiperidin-1- yl)pyrido[3,4- d]pyrimidin-4-amine | 8.44 (s, 1H), 8.29 (d, J = 6.8 Hz, 1H), 7.46 (s, 1H), 6.83 (s, 1H), 6.80 (s, 1H), 6.69 (s, 1H), 5.54 (s, 2H), 5.27 (quin, J = 6.8 Hz, 1H), 3.95 (q, J = 7.6 Hz, 2H), 3.84 (q, J = 7.6 Hz, 2H), 3.67 (t, J = 5.6 Hz, 4H), 2.28-2.17 (m, 2H), 2.09-2.02 (m, 4H), 1.54 (d, J = 6.8 Hz, 3H) 508.20 [M + H]⁺ |
| 52 | 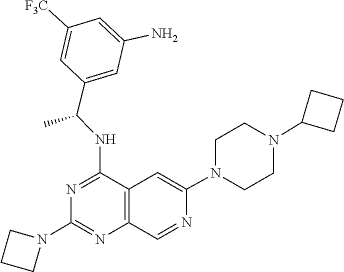 [SMILES: C[C@@H](Nc1nc(N2CCC2)nc2cnc(N3CCN(C4CCC4)CC3)cc12)c1cc(N)cc(C(F)(F)F)c1] | (R)-N-(1-(3-amino-5- (trifluoromethyl) phenyl)ethyl)-2- (azetidin- 1-yl)-6-(4- cyclobutylpiperazin-1- yl)pyrido[3,4- d]pyrimidin-4-amine | (DMSO-d₆ + D₂O): δ 8.44 (s, 1H), 7.39 (s, 1H), 6.87 (s, 1H), 6.85 (s, 1H), 6.73 (s, 1H), 5.24 (q, J = 7.2 Hz, 1H), 4.03-3.97 (m, 2H), 3.88 (2H merged with moisture peak), 3.54 (bs, 4H), 3.20 (bs, 1H), 2.70 (bs, 4H), 2.24-2.21 (m, 2H), 2.14-2.06 (m, 2H), 2.01- 2.19 (m, 2H), 1.74-1.70 (m, 2H), 1.55 (d, J = 7.2 Hz, 3H) 527.10 [M + H]⁺. |
| 53 | 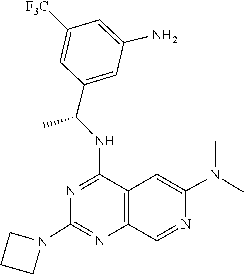 [SMILES: C[C@@H](Nc1nc(N2CCC2)nc2cnc(N(C)C)cc12)c1cc(N)cc(C(F)(F)F)c1] | (R)-N4-(1-(3-amino- 5- (trifluoromethyl) phenyl)ethyl)-2- (azetidin- 1-yl)-N6,N6- dimethylpyrido[3,4- d]pyrimidine-4,6- diamine | 8.38 (s, 1H), 8.27 (d, J = 6.8 Hz, 1H), 7.15 (s, 1H), 6.84 (s, 1H), 6.81 (s, 1H), 6.68 (s, 1H), 5.53 (s, 2H), 5.27 (quin, J = 7.2 Hz, 1H), 3.93 (q, J = 7.6 Hz, 2H), 3.83 (q, J = 7.6 Hz, 2H), 3.05 (s, 6H), 2.21-2.14 (m, 2H), 1.54 (d, J = 7.2 Hz, 3H) 432.20 [M + H]⁺ |
| 54 | 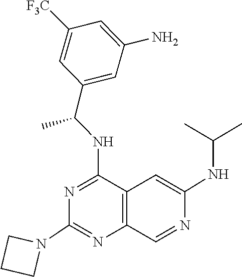 [SMILES: CC(C)Nc1cc2c(N[C@H](C)c3cc(N)cc(C(F)(F)F)c3)nc(N3CCC3)nc2cn1] | (R)-N4-(1-(3-amino- 5- (trifluoromethyl) phenyl)ethyl)-2- (azetidin- 1-yl)-N6- isopropylpyrido[3,4- d]pyrimidine-4,6- diamine | 8.29 (s, 1H), 6.97 (bs, 1H), 6.85 (s, 1H), 6.80 (s, 1H), 6.69 (s, 1H), 5.53 (s, 2H), 5.25-5.24 (m, 1H), 3.95 (bs, 2H), 3.90-3.83 (m, 5H), 2.22-2.16 (m, 2H), 1.53 (d, J = 6.8 Hz, 3H), 1.18 (d, J = 5.6 Hz, 6H) 446.20 [M + H]⁺ |
| 55 | 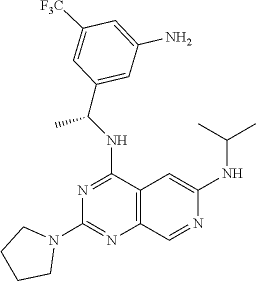 [SMILES: CC(C)Nc1cc2c(N[C@H](C)c3cc(N)cc(C(F)(F)F)c3)nc(N3CCCC3)nc2cn1] | (R)-N4-(1-(3-amino- 5- (trifluoromethyl) phenyl)ethyl)-N6- isopropyl-2- (pyrrolidin-1- yl)pyrido[3,4- d]pyrimidine-4,6- diamine | 8.38 (bs, 1H), 7.07 (bs, 1H), 6.87 (s, 1H), 6.81 (s, 1H), 6.69 (s, 1H), 5.56 (bs, 2H), 5.38-5.20 (m, 1H), 3.86 (bs, 1H), 3.50-3.56 (m, 4H), 3.00-2.90 (m, 1H), 1.91-1.89 (m, 4H), 1.56 (d, J = 6.8 Hz, 3H), 1.19 (t, J = 5.2 Hz, 6H) (One exchangeable proton not observed in ¹H-NMR) 460.40 [M + H]⁺ |
| 56 | 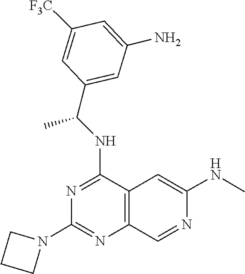 [SMILES: CNc1cc2c(N[C@H](C)c3cc(N)cc(C(F)(F)F)c3)nc(N3CCC3)nc2cn1] | (R)-N4-(1-(3-amino- 5- (trifluoromethyl) phenyl)ethyl)-2- (azetidin- 1-yl)-N6- methylpyrido[3,4- d]pyrimidine-4,6- diamine | 8.29 (s, 1H), 8.21 (d, J = 7.2 Hz, 1H), 6.91 (s, 1H), 6.85 (s, 1H), 6.81 (s, 1H), 6.68 (s, 1H), 6.01 (q, J = 5.2 Hz, 1H), 5.52 (s, 2H), 5.25 (quin, J = 7.2 Hz, 1H), 3.92 (q, J = 7.6 Hz, 2H), 3.82 (q, J = 7.6 Hz, 2H), 2.80 (d, J = 5.2 Hz, 3H), 2.17 (quin, J = 7.2 Hz, 2H), 1.53 (d, J = 7.2 Hz, 3H) 418.30 [M + H]⁺ |

TABLE 4
Compounds in table-4 were prepared by using process analogous to the
preparation of example-3

| | | | 1H NMR (400 MHz, |
|---|---|---|---|
| | | | DMSO-d₆): δ ppm/LC- |
| Example # | Structure | IUPAC name | MS: m/z |
| 57 | 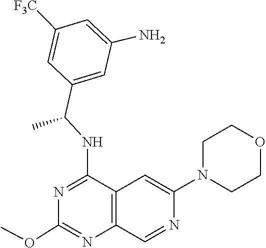 [SMILES: COc1nc(N[C@H](C)c2cc(N)cc(C(F)(F)F)c2)c2cc(N3CCOCC3)ncc2n1] | (R)-N-(1-(3-amino-5- (trifluoromethyl) phenyl)ethyl)-2- methoxy-6- morpholinopyrido [3,4-d]pyrimidin- 4-amine | 8.60 (s, 1H), 8.56 (d, J = 7.6 Hz, 1H), 7.49 (s, 1H), 6.82 (s, 1H), 6.79 (s, 1H), 6.70 (s, 1H), 5.58 (bs, 2H), 5.42- 5.38 (m, 1H), 3.80 (s, 3H), 3.78 (t, J = 4.8 Hz, 4H), 3.47 (t, J = 4.8 Hz, 4H), 1.55 (d, J = 6.8 Hz, 3H) 449.50 [M + H]⁺ |
| 58 | 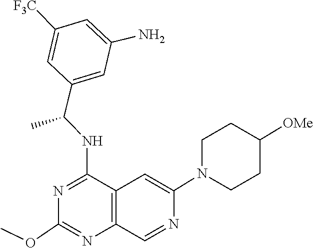 [SMILES: COc1nc(N[C@H](C)c2cc(N)cc(C(F)(F)F)c2)c2cc(N3CCC(OC)CC3)ncc2n1] | (R)-N-(1-(3-amino-5- (trifluoromethyl) phenyl)ethyl)-2- methoxy-6-(4- methoxypiperidin-1- yl)pyrido[3,4- d]pyrimidin-4-amine | 8.56 (s, 1H), 8.53 (bs, 1H), 7.48 (s, 1H), 6.82 (s, 1H), 6.79 (s, 1H), 6.70 (s, 1H), 5.57 (bs, 2H), 5.40 (quin, J = 7.2 Hz, 1H), 4.02-3.88 (m, 2H), 3.79 (s, 3H), 3.44- 3.41 (m, 1H), 3.30 (s, 3H), 3.21-3.15 (m, 2H), 1.98- 1.95 (m, 2H), 1.55 (d, J = 7.2 Hz, 3H), 1.51-1.47 (m, 2H) 477.40 [M + H]⁺ |

### Example—59: (R)-2-chloro-N-(1-(3-(difluoromethyl)-2-fluorophenyl)ethyl)-6-morpholinopyrido[3,4-d]pyrimidin-4-amine

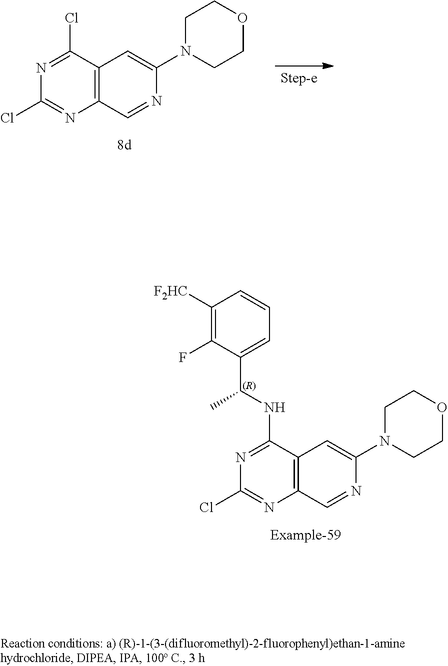

To a solution of 4-(2,4-dichloropyrido[3,4-d]pyrimidin-6-yl)morpholine (100 mg, 0.558 mmol) in IPA (5 mL) were added DIPlA (0.2 mL, 1.052 mmol) and (R)-1-(3-(difluoromethyl)-2-fluorophenyl)ethan-1-amine hydrochloride (79 mg, 0.358 mmol). The reaction mixture was stirred at 80° C. for 3 h. Reaction progress was monitored by TLC. The reaction mixture was diluted with water (10 mL), extracted with EtOAc (3×20 mL). The combined organic layers were washed with brine (20 mL), dried over anhydrous sodium sulfate and concentrated under reduced pressure. The residue was purified by flash column chromatography with 4% methanol in DCM as an eluent to afford the title compound as a yellow solid. Yield: 70 mg (52%); ¹H-NMR (400 MHz, DMSO-d₆): δ 8.99 (d, J=7.2z, 1H), 8.70 (s, 1H), 7.66 (t, J 7.2 Hz, 1H), 7.55 (t, J=7.6 Hz, 1H), 7.1 (s, 1H), 7.33 (t, J=d_{7.6}Hi-z, 1H), 7.24 (t, J J 54.4 Hz, 1H), 5.72-0.68 (m, 1H), 3.78 (t, J 4.4 Hz, 4H), 3.55 (t, J 4.4 Hz, 4H), 1.63 (d, Jq 6.8 Hz, 3H); LCMS: m/z 438.20 [M+H]⁺.

TABLE 5
Compounds in table-5 were prepared by using process analogous to the
preparation of example-59

| | | | 1H NMR (400 MHz, |
|---|---|---|---|
| Example | | | DMSO-d₆): δ ppm/LC- |
| # | Structure | IUPAC name | MS:m/z |
| 60 | 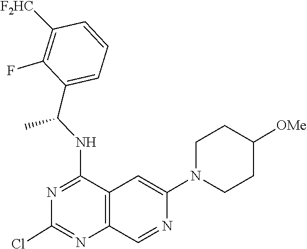 [SMILES: COC1CCN(c2cc3c(N[C@H](C)c4cccc(C(F)F)c4F)nc(Cl)nc3cn2)CC1] | (R)-2-chloro-N-(1-(3- (difluoromethyl)-2- fluorophenyl)ethyl)-6- (4-methoxypiperidin-1- yl)pyrido[3,4- d]pyrimidin-4-amine | 8.94 (d, J = 7.2 Hz, 1H), 8.66 (s, 1H), 7.66 (t, J = 6.8 Hz, 1H), 7.54 (t, J = 6.8 Hz, 1H), 7.49 (s, 1H), 7.33 (t, J = 7.6 Hz, 1H), 7.24 (t, J = 54.4 Hz, 1H), 5.70 (quin, J = 6.8 Hz, 1H), 4.05- 4.02 (m, 2H), 3.48-3.44 (m, 1H), 3.26 (bs, 2H), 3.30 (3H merged with DMSO moisture peak), 2.0-1.92 (m, 2H), 1.63 (d, J = 6.8 Hz, 3H), 1.54-1.48 (m, 2H) 466.30 [M + H]⁺ |
| 61 | 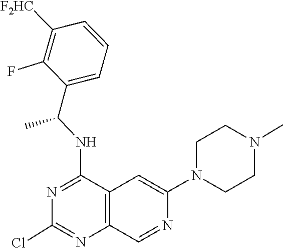 [SMILES: C[C@@H](Nc1nc(Cl)nc2cnc(N3CCN(C)CC3)cc12)c1cccc(C(F)F)c1F] | (R)-2-chloro-N-(1-(3- (difluoromethyl)-2- fluorophenyl)ethyl)-6- (4-methylpiperazin-1- yl)pyrido[3,4- d]pyrimidin-4-amine | 8.97 (d, J = 7.6 Hz, 1H), 8.67 (s, 1H), 7.66 (t, J = 6.8 Hz, 1H), 7.55 (t, J = 6.8 Hz, 1H), 7.49 (s, 1H), 7.33 (t, J = 7.6 Hz, 1H), 7.24 (t, J = 54.4 Hz, 1H), 5.68 (quin, J = 7.2 Hz, 1H), 3.59 (t, J = 4.8 Hz, 4H), 2.47 (t, J = 4.8 Hz, 4H), 2.25 (s, 3H), 1.63 (d, J = 7.2 Hz, 3H) 451.10 [M + H]⁺ |
| 62 | 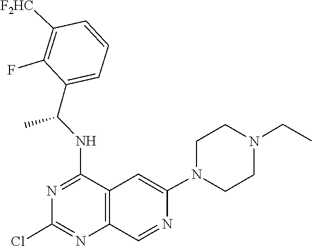 [SMILES: CCN1CCN(c2cc3c(N[C@H](C)c4cccc(C(F)F)c4F)nc(Cl)nc3cn2)CC1] | (R)-2-chloro-N-(1-(3- (difluoromethyl)-2- fluorophenyl)ethyl)-6- (4-ethylpiperazin-1- yl)pyrido[3,4- d]pyrimidin-4-amine | 8.99 (d, J = 7.2 Hz, 1H), 8.68 (s, 1H), 7.66 (t, J = 7.6 Hz, 1H), 7.55 (t, J = 7.6 Hz, 1H), 7.49 (s, 1H), 7.33 (t, J = 7.6 Hz, 1H), 7.25 (t, J = 54.4 Hz, 1H), 5.69 (quin, J = 7.6 Hz, 1H), 3.59 (bs, 4H), 2.50 (4H merged with DMSO peak), 2.41 (q, J = 6.8 Hz, 2H), 1.63 (d, J = 7.2 Hz, 3H), 1.06 (t, J = 6.8 Hz, 3H) 465.10 [M + H]⁺ |

### Example—63: Synthesis of (R)—N-(1-(3-amino-5-(trifluoromethyl)phenyl)ethyl)-2-chloro-6-(tetrahydro-2H-pyran-4-yl)pyrido[3,4-d]pyrimidin-4-amine

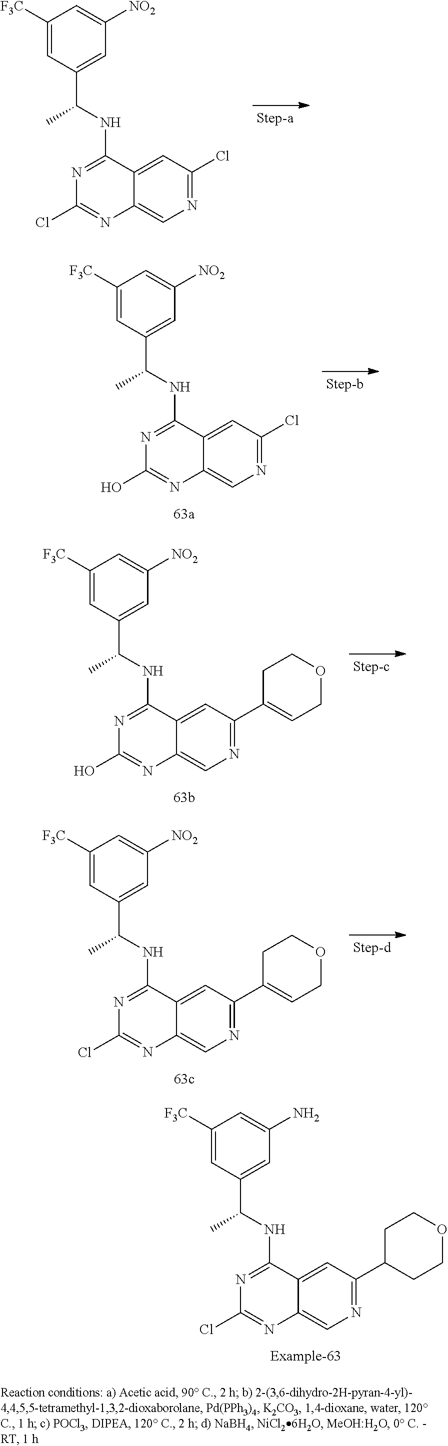 [SMILES: C[C@@H](Nc1nc(Cl)nc2cnc(Cl)cc12)c1cc([N+](=O)[O-])cc(C(F)(F)F)c1 ; C[C@@H](Nc1nc(O)nc2cnc(Cl)cc12)c1cc([N+](=O)[O-])cc(C(F)(F)F)c1 ; C[C@@H](Nc1nc(O)nc2cnc(C3=CCOCC3)cc12)c1cc([N+](=O)[O-])cc(C(F)(F)F)c1 ; C[C@@H](Nc1nc(Cl)nc2cnc(C3=CCOCC3)cc12)c1cc([N+](=O)[O-])cc(C(F)(F)F)c1 ; C[C@@H](Nc1nc(Cl)nc2cnc(C3CCOCC3)cc12)c1cc(N)cc(C(F)(F)F)c1]

### Step—a: (R)-6-chloro-4-((1-(3-nitro-5-(trifluoromethyl)phenyl)ethyl)amino)pyrido[3,4-d]pyrimidin-2-ol (63a)

A solution of (R)-2,6-dichloro-N-(1-(3-nitro-5-(trifluoromethyl)phenyl)ethyl)pyrido[3,4-d]pyrimidin-4-amine (500 mg, 1.16 mmol) in acetic acid (10 mL) was stirred at 90° C. for 2 h. Progress of the reaction was monitored by TLC. The reaction mixture was concentrated under reduced pressure, the residue was diluted with water (50 mL) and the precipitated solid was filtered and dried under vacuum to afford the title compound as a yellow solid. Yield: 430 mg (90%); ¹H-NMR (400 MHz, DMSO-d₆): δ ppm 11.09 (bs, 1H), 8.90 (d, J=7.2 Hz, 1H), 8.58 (bs, 1H), 8.39 (s, 1H), 8.33 (s, 1H), 8.32 (s, 1H), 8.30 (s, 1H), 5.64 (quin, J=6.8 Hz, 1H), 1.61 (d, J=7.2 Hz, 3H); LCMS: m/z 414.0 [M+H]⁺.

### Step—b: (R)-6-(3,6-dihydro-2H-pyran-4-yl)-4-((1-(3-nitro-5-(trifluoromethyl)phenyl)ethyl)amino)pyrido[3,4-d]pyrimidin-2-ol (63b)

To a solution of (R)-6-chloro-4-((1-(3-nitro-5-(trifluoromethyl)phenyl)ethyl)amino)pyrido[3,4-d]pyrimidin-2-ol (430 mg, 1.04 mmol) and 2-(3,6-dihydro-2H-pyran-4-yl)-4,4,5,5-tetramethyl-1,3,2-dioxaborolane (328 mg, 1.56 mmol) in 1,4-dioxane (6 mL) and water (2 mL) was added potassium carbonate (431 mg, 3.12 mmol). The reaction mixture was purged with nitrogen gas for 2 min, followed by the addition of Tetrakis(triphenylphosphine)palladium(0) (120 mg, 0.104 mmol) the microwave vial was capped and irradiated at 120° C. for 1 h in microwave reactor. The reaction mixture was diluted with ethyl acetate (30 mL), filtered through celite pad, then the filtrate was washed with water (20 mL). The aqueous layer was extracted with ethyl acetate (30 mL), the combined organic layer was washed with brine (30 mL), dried over anhydrous sodium sulfate and evaporated under reduced pressure. The residue was purified by flash column chromatography using 10% methanol in DCM as an eluent to afford the title compound as a cream colour solid. Yield: 420 mg (88%); LCMS: m/z 462.10 [M+H]⁺.

### Step—c: (R)-2-chloro-6-(3,6-dihydro-2H-pyran-4-yl)-N-(1-(3-nitro-5-(trifluoromethyl)phenyl)ethyl)pyrido[3,4-d]pyrimidin-4-amine (63c)

To a solution of (R)-6-(3,6-dihydro-2H-pyran-4-yl)-4-((1-(3-nitro-5-(trifluoromethyl)phenyl)ethyl)amino)pyrido[3,4-d]pyrimidin-2-ol (420 mg, 0.91 mmol) in phosphorus(V) oxychloride (5 mL) was added diisopropylethylamine (0.5 mL) at RT and the reaction mixture was stirred at 120° C. for 2 h. The progress of the reaction was monitored by TLC. Phosphorus(V)oxychloride was distilled off under reduced pressure. Obtained residue was washed with diethyl ether (3×50 mL) and decanted. The diethyl ether layer was washed with saturated sodium bicarbonate solution and brine solution (50 mL), dried over anhydrous sodium sulfate and concentrated under reduced pressure to afford the title compound as a brown solid. Yield: 205 mg (49%); LCMS: m/z 480.10 [M+H]⁺.

### Step—d: (R)—N-(1-(3-amino-5-(trifluoromethyl)phenyl)ethyl)-2-chloro-6-(tetrahydro-2H-pyran-4-yl)pyrido[3,4-d]pyrimidin-4-amine

The title compound was synthesized using the similar procedure followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 31%; ¹H-NMR (400 MHz, DMSO-d₆): δ 9.20 (d, J=7.6 Hz, 1H), 8.97 (s, 1H), 8.23 (s, 1H), 6.88 (s, 1H), 6.82 (s, 1H), 6.74 (s, 1H), 5.61 (bs, 2H), 5.45 (quin, J=7.2 Hz, 1H), 4.02-3.99 (m, 2H), 3.53-3.46 (m, 2H), 3.09-3.03 (m, 1H), 1.90-1.80 (m, 4H), 1.58 (d, J=7.2 Hz, 3H); LCMS: m/z 452.10 [M+H]⁺.

TABLE 6
Example-64 was prepared by using process analogous to the preparation of
example-63

| | | | 1H NMR (400 MHz, |
|---|---|---|---|
| Example | | | DMSO- d₆): δppm/ |
| # | Structure | IUPAC Name | LC-MS: m/z |
| 64 | 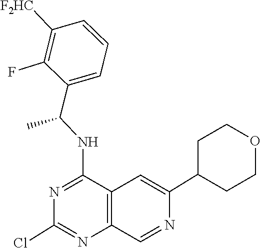 [SMILES: C[C@@H](Nc1nc(Cl)nc2cnc(C3CCOCC3)cc12)c1cccc(C(F)F)c1F] | (R)-2-chloro-N-(1-(3- (difluoromethyl)-2- fluorophenyl)ethyl)-6- (tetrahydro-2H-pyran- 4-yl)pyrido[3,4- d]pyrimidin-4-amine | 9.28 (s, 1H), 8.97 (s, 1H), 8.26 (s, 1H), 7.70 (t, J = 6.8 Hz, 1H), 7.55 (t, J = 6.8 Hz, 1H), 7.38- 7.32 (m, 1H), 7.24 (t, J = 54.4 Hz, 1H), 5.78- 5.71 (m, 1H), 4.03-3.99 (m, 2H), 3.53-3.47 (m, 2H), 3.10-3.06 (m, 1H), 1.91-1.82 (m, 4H), 1.64 (d, J = 7.2 Hz, 3H) 437.20 [M + H]⁺ |

### Example—65: (R)—N4-(1-(3-amino-5-(trifluoromethyl)phenyl)ethyl)-N2,N2-dimethyl-6-(tetrahydro-2H-pyran-4-yl)pyrido[3,4-d]pyrimidine-2,4-diamine

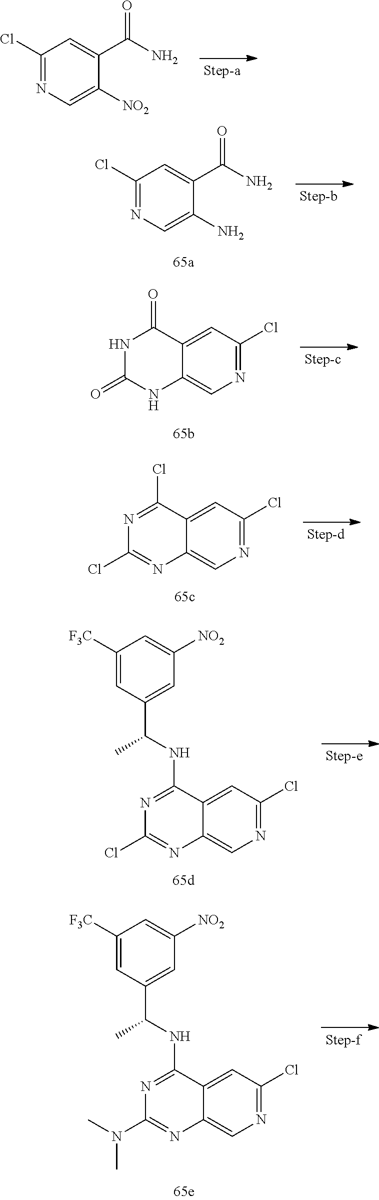 [SMILES: NC(=O)c1cc(Cl)ncc1[N+](=O)[O-] ; NC(=O)c1cc(Cl)ncc1N ; O=c1[nH]c(=O)c2cc(Cl)ncc2[nH]1 ; Clc1cc2c(Cl)nc(Cl)nc2cn1 ; C[C@@H](Nc1nc(Cl)nc2cnc(Cl)cc12)c1cc([N+](=O)[O-])cc(C(F)(F)F)c1 ; C[C@@H](Nc1nc(N(C)C)nc2cnc(Cl)cc12)c1cc([N+](=O)[O-])cc(C(F)(F)F)c1]

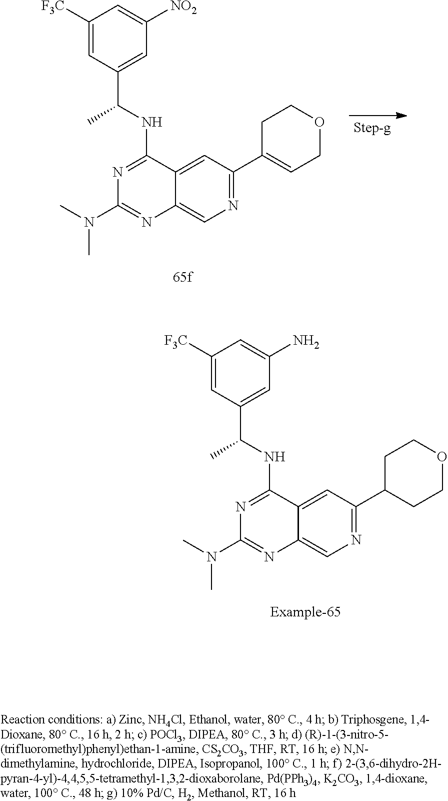 [SMILES: C[C@@H](Nc1nc(N(C)C)nc2cnc(C3=CCOCC3)cc12)c1cc([N+](=O)[O-])cc(C(F)(F)F)c1 ; C[C@@H](Nc1nc(N(C)C)nc2cnc(C3CCOCC3)cc12)c1cc(N)cc(C(F)(F)F)c1]

### Step—a: 5-amino-2-chloroisonicotinamide (65a)

To a stirred solution of 2-chloro-5-nitroisonicotinamide (4.0 g, 0.0199 mmol) in ethanol (40 mL) and water (10 mL) at ambient temperature was added ammonium chloride (10.6 g, 0.199 mmol) and Zinc (6.5 g, 0.0995 mmol). The reaction mixture was heated at 80° C. for 4 h. The reaction mixture was cooled to RT, diluted with ethyl acetate (50 mL), filtered through celite pad. The filtrate was concentrated under reduced pressure, the residue was diluted with ethyl acetate (100 mL), water (50 mL) and extracted. Aqueous layer was extracted with ethyl acetate (100 mL), the combined organic layers were washed with brine solution (50 mL), dried over Na₂SO₄ and concentrated under reduced pressure. The residue was purified by flash column chromatography using 5% MeOH in DCM as an eluent to afford the title compound as a brown solid (Yield: 1.8 g, 42%); LCMS: m/z 171.9 [M+H]⁺.

### Step—b: 6-chloropyrido[3,4-d]pyrimidine-2,4(1H,3H)-dione (65b)

To a solution of 5-amino-2-chloroisonicotinamide (1.8 g, 10.4 mmol) was in 1,4-dioxane (15 mL) was added triphosgene (4.6 g, 15.7 mmol) in portions. The reaction mixture was stirred at 80° C. for 2 h and the reaction progress was monitored by TLC. Upon consumption of starting material, 1,4-dioxane was distilled off under reduced pressure and the residue was triturated with ethyl acetate. The resulting solid was filtered, washed with ethyl acetate and dried under vacuum to afford the title compound as a yellow solid. Yield: 1.6 g (76%); LCMS: m/z 198.0 [M+H]⁺.

### Step—c: 2,4,6-trichloropyrido[3,4-d]pyrimidine (65c)

To a solution of 6-chloropyrido[3,4-d]pyrimidine-2,4(1H,3H)-dione (600 mg, 3.03 mmol) in phosphorus(V) oxychloride (10 mL) was added diisopropylethylamine (1.6 mL, 9.11 mmol) at RT and the reaction mixture was stirred at 120° C. for 3 h. The progress of the reaction was monitored by TLC. Phosphorus(V)oxychloride was distilled off under reduced pressure. Obtained residue was washed with diethyl ether (3×50 mL) and decanted. The diethyl ether layer was washed with saturated sodium bicarbonate solution and brine solution (50 mL), dried over anhydrous sodium sulfate and concentrated under reduced pressure to afford 2,4,6-trichloropyrido[3,4-d]pyrimidine as a yellow solid. Yield: 600 mg (84%); LCMS: m/z 233.9 [M+H]⁺.

### Step—d: (R)-2,6-dichloro-N-(1-(3-nitro-5-(trifluoromethyl)phenyl)ethyl)pyrido[3,4-d]pyrimidin-4-amine (65d)

To a solution of 2,4,6-trichloropyrido[3,4-d]pyrimidine (600 mg, 2.56 mmol) in THF (15 mL) were added cesium carbonate (2.1 g, 6.39 mmol) and (R)-1-(3-nitro-5-(trifluoromethyl)phenyl)ethan-1-amine hydrochloride (554 mg, 2.047 mmol). The reaction mixture was stirred at room temperature for 16 h. Reaction progress was monitored by TLC. The reaction mixture was diluted with water (20 mL) and extracted with EtOAc (3×30 mL). The combined organic extracts were washed with brine (20 mL), dried over anhydrous sodium sulfate and concentrated under reduced pressure. The residue obtained was purified by flash column chromatography using 30% Ethyl acetate in hexane as an eluent to afford the title compound as a brown solid. Yield: 200 mg (56%); LCMS: m/z 432.1 [M+H]⁺.

### Step—e: (R)-6-chloro-N2,N2-dimethyl-N4-(1-(3-nitro-5-(trifluoromethyl)phenyl)ethyl)pyrido[3,4-d]pyrimidine-2,4-diamine (65e)

To a solution of (R)-2,6-dichloro-N-(1-(3-nitro-5-(trifluoromethyl)phenyl)ethyl)pyrido[3,4-d]pyrimidin-4-amine (100 mg, 0.241 mmol) in isopropanol (3 mL) were added DIPEA (0.2 mL, 1.45 mmol) and N,N-dimethylamine hydrochloride (59 mg, 0.725 mmol). The microwave vial was capped and irradiated in microwave reactor at 100° C. for 1 h. The reaction mixture was diluted with water (20 mL) and extracted with EtOAc (2×30 mL). The combined organic extracts were washed with brine (20 mL), dried over anhydrous sodium sulfate and concentrated under reduced pressure to afford the title compound as a brown solid. Yield: 100 mg (94%); LCMS: m/z 441.1 [M+H]⁺.

### Step—f: (R)-6-(3,6-dihydro-2H-pyran-4-yl)-N2,N2-dimethyl-N4-(1-(3-nitro-5-(trifluoromethyl)phenyl)ethyl)pyrido[3,4-d]pyrimidine-2,4-diamine (65f)

To a solution of (R)-6-chloro-N2,N2-dimethyl-N4-(1-(3-nitro-5-(trifluoromethyl)phenyl)ethyl)pyrido[3,4-d]pyrimidine-2,4-diamine (100 mg, 0.227 mmol) and 2-(3,6-dihydro-2H-pyran-4-yl)-4,4,5,5-tetramethyl-1,3,2-dioxaborolane (52 mg, 0.25 mmol) in 1,4-dioxane (4 mL) and water (1 mL) was added potassium carbonate (94 mg, 0.68 mmol). The reaction mixture was purged with nitrogen gas for 2 min, followed by the addition of Tetrakis(triphenylphosphine)palladium(0) (13 mg, 0.011 mmol) the reaction mixture was stirred at 100° C. for 48 h. The reaction mixture was cooled to RT, diluted with water (20 mL) and extracted with ethyl acetate (2×20 mL). The combined organic extracts were washed with brine solution (10 mL), dried over anhydrous sodium sulfate and evaporated under reduced pressure. The residue was purified by flash column chromatography using 4% methanol in DCM as an eluent to afford the title compound as a yellow solid. Yield: 100 mg (90%); LCMS: m/z 489.20 [M+H]⁺.

### Step—g: (R)—N4-(1-(3-amino-5-(trifluoromethyl)phenyl)ethyl)-N2,N2-dimethyl-6-(tetrahydro-2H-pyran-4-yl)pyrido[3,4-d]pyrimidine-2,4-diamine

To a stirred solution of (R)-6-(3,6-dihydro-2H-pyran-4-yl)-N2,N2-dimethyl-N4-(1-(3-nitro-5-(trifluoromethyl)phenyl)ethyl)pyrido[3,4-d]pyrimidine-2,4-diamine (100 mg, 0.205 mmol) in methanol (20 mL) was added slurry of 10% Pd—C(50% wet, 30 mg) in methanol (2 mL) and the reaction mixture was stirred under hydrogen atmosphere for 16 h. Reaction progress was monitored by TLC. The reaction mixture was filtered through celite pad, washed with methanol (20 mL) and the filtrate was evaporated under reduced pressure. The residue was purified by prep-HPLC to afford the title compound (Yield: 6 mg, 6%); H-NMR (400 MHz, DMSO-d₆): δ ppm 8.58 (s, 1H), 8.40 (d, J 7.2 Hz, 1H), 7.94 (s, 1H), 6.86 (s, 1H), 6.81 (s, 1H), 6.68 (s, 1H), 5.53 (bs, 2H), 5.25 (quin, J 7.2 Hz, 1H), 4.02-3.95 (i, 2H), 3.50-3.46 (i, 2H), 3.04 (s, 6H), 2.95-2.90 (m, 1H), 1.84-1.81 (m, 4H), 1.54 (d, J=6.8 Hz, 3H); LCMS: m/z 461.20 [M+H].

TABLE 7
Compounds in table-7 were prepared by using process analogous to the
preparation of example-65

| | | | 1H NMR (400 MHz, |
|---|---|---|---|
| | | | DMSO-d₆): δppm/ |
| Example # | Structure | IUPAC Name | LC-MS: m/z |
| 66 | 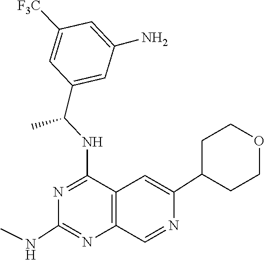 [SMILES: CNc1nc(N[C@H](C)c2cc(N)cc(C(F)(F)F)c2)c2cc(C3CCOCC3)ncc2n1] | (R)-N4-(1-(3-amino-5- (trifluoromethyl)phenyl) ethyl)-N2-methyl-6- (tetrahydro-2H-pyran-4- yl)pyrido[3,4- d]pyrimidine-2,4- diamine | 8.60 (bs, 1H), 7.95 (s, 1H), 6.84 (s, 1H), 6.81 (s, 1H), 6.82 (s, 1H), 6.70 (s, 1H), 5.55 (s, 2H), 5.43 (bs, 1H), 4.0-3.97 (m, 2H), 3.50- 3.44 (m, 2H), 2.96-2.89 (m, 1H), 2.78 (d, J = 4.4 Hz, 3H), 1.83-1.78 (m, 4H), 1.54 (d, J = 6.8 Hz, 3H) (One exchangeable proton not observed in ¹H-NMR) 447.20 [M + H]⁺ |
| 67 | 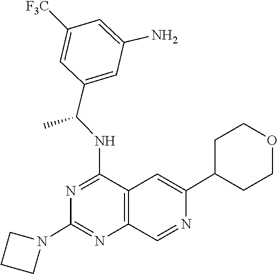 [SMILES: C[C@@H](Nc1nc(N2CCC2)nc2cnc(C3CCOCC3)cc12)c1cc(N)cc(C(F)(F)F)c1] | (R)-N-(1-(3-amino-5- (trifluoromethyl)phenyl) ethyl)-2-(azetidin-1-yl)-6- (tetrahydro-2H-pyran-4- yl)pyrido[3,4- d]pyrimidin-4-amine | 8.61 (s, 1H), 8.44 (bs, 1H), 7.94 (s, 1H), 6.85 (s, 1H), 6.81 (s, 1H), 6.69 (s, 1H), 5.53 (s, 2H), 5.26 (quin, J = 6.8 Hz, 1H), 4.04-3.96 (m, 4H), 3.91 (bs, 2H), 3.49- 3.43 (m, 2H), 2.95-2.89 (m, 1H), 2.24-2.18 (m, 2H), 1.82-1.78 (m, 4H), 1.54 (d, J = 7.2 Hz, 3H) 473.20 [M + H]⁺ |
| 68 | 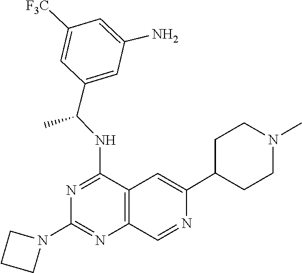 [SMILES: C[C@@H](Nc1nc(N2CCC2)nc2cnc(C3CCN(C)CC3)cc12)c1cc(N)cc(C(F)(F)F)c1] | (R)-N-(1-(3-amino-5- (trifluoromethyl)phenyl) ethyl)-2-(azetidin-1-yl)-6- (1-methylpiperidin-4- yl)pyrido[3,4- d]pyrimidin-4-amine | (DMSO-d6 + D2O) 8.62 (d, J = 3.6 Hz, 1H), 8.38 (s, 1H), 7.91 (s, 1H), 6.93 (s, 1H), 6.90 (s, 1H), 6.77 (s, 1H), 5.20 (bs, 1H), 4.05 (q, J = 7.2 Hz, 2H), 3.92 (bs, 2H), 3.37 (bs, 2H), 23.0-2.80 (m, 3H), 2.68 (s, 3H), 2.30- 2.20 (m, 2H), 2.15-2.05 (m, 2H), 2.0-1.90 (m, 2H), 1.56 (d, J = 6.4 Hz, 3H) (Two exchangeable protons not observed in ¹H-NMR) 486.10 [M + H]⁺ |
| 69 | 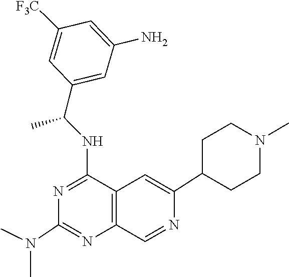 | (R)-N4-(1-(3-amino-5- (trifluoromethyl)phenyl) ethyl)-N2,N2-dimethyl-6- (1-methylpiperidin-4- yl)pyrido[3,4- d]pyrimidine-2,4- diamine | 8.57 (s, 1H), 8.38 (d, J = 6.8 Hz, 1H), 7.94 (s, 1H), 6.86 (s, 1H), 6.80 (s, 1H), 6.68 (s, 1H), 5.53 (s, 2H), 5.26- 5.22 (m, 1H), 3.04 (s, 6H), 2.90 (d, J = 10.8 Hz, 2H), 2.50 (1H merged with DMSO peak), 2.33 (s, 3H), 2.02-1.94 (m, 2H), 1.86- 1.81 (m, 4H), 1.54 (d, J = 6.8 Hz, 3H) 474.0 [M + H]⁺ |

### Example—70: (R)—N-(1-(3-amino-5-(trifluoromethyl)phenyl)ethyl)-2-chloro-6-(4-ethylpiperazin-1-yl)-7-fluoroquinazolin-4-amine

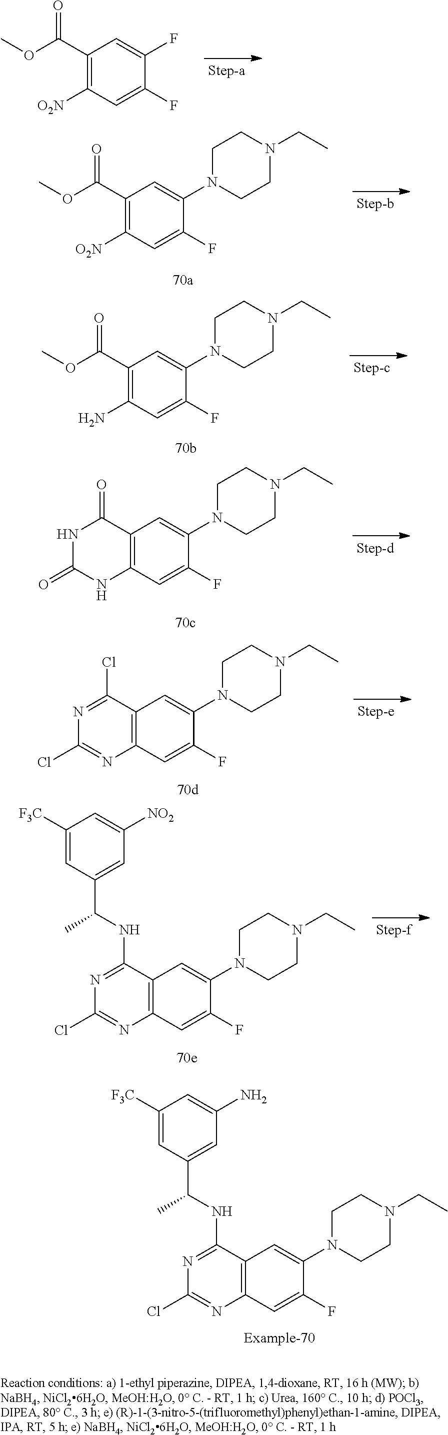

### Step—a: methyl 5-(4-ethylpiperazin-1-yl)-4-fluoro-2-nitrobenzoate (70a)

To a solution of methyl 4,5-difluoro-2-nitrobenzoate (1.9 g, 8.75 mmol) in 1,4-dioxane (40 mL) were added N,N-diisopropylethylamine (3.13 mL, 17.5 mmol) and 1-ethyl piperazine (1.34 mL, 10.5 mmol) under nitrogen atmosphere at room temperature. The reaction mixture was stirred at RT for 16 h. Progress of the reaction was monitored by TLC. After completion of reaction, 1,4-dioxane was distilled off under reduced pressure. The residue was extracted with DCM (50 mL) and water (30 mL), aqueous layer was again extracted with DCM (3×50 mL). The combined organic layers were washed with brine solution (30 mL), dried over anhydrous sodium sulphate and concentrated under reduced pressure. The residue was purified by flash column chromatography using 2.5% MeOH in DCM as an eluent to afford the title compound as a yellow solid (Yield: 1.7 g, 62%); LCMS: m/z 312.0 [M+H]⁺.

### Step—b: methyl 2-amino-5-(4-ethylpiperazin-1-yl)-4-fluorobenzoate (70b)

The title compound was synthesized using the similar procedure which was followed for 5-Amino-2-(pyrrolidin-1-yl)isonicotinamide (1c); Yield: 18%; LCMS: m/z 282.0 [M+H]⁺.

### Step—c: 6-(4-ethylpiperazin-1-yl)-7-fluoroquinazoline-2,4(1H,3H)-dione (70c)

A mixture of methyl 2-amino-5-(4-ethylpiperazin-1-yl)-4-fluorobenzoate (340 mg, 1.20 mmol) and urea (725 mg, 12.0 mmol) was heated at 160° C. for 10 h. The reaction mixture was cooled to RT, diluted with water (20 ml) and precipitated solid was filtered and dried under vacuum. The solid obtained was purified by flash column chromatography using 7% methanol in DCM as an eluent to afford the title compound as a white solid (Yield: 120 mg, 34%); LCMS: m/z 293.0 [M+H]⁺.

### Step—d: 2,4-dichloro-6-(4-ethylpiperazin-1-yl)-7-fluoroquinazoline (70d)

The title compound was synthesized using the similar procedure followed for 2,4-Dichloro-6-(pyrrolidin-1-yl)pyrido[3,4-d]pyrimidine (le); Yield: 74%; LCMS: m/z 328.90 [M+H]⁺.

### Step—e: (R)-2-chloro-6-(4-ethylpiperazin-1-yl)-7-fluoro-N-(1-(3-nitro-5-(trifluoromethyl)phenyl)ethyl)quinazolin-4-amine (70e)

The title compound was synthesized using the similar procedure followed for R)-2-Chloro-N-(1-(3-nitro-5-(trifluoromethyl)phenyl)ethyl)-6-(pyrrolidin-1-yl)pyrido[3,4-d]pyrimidin-4-amine (if); Yield: 37%; LCMS: m/z 527.10 [M+H]⁺.

### Step—f: (R)—N-(1-(3-amino-5-(trifluoromethyl)phenyl)ethyl)-2-chloro-6-(4-ethylpiperazin-1-yl)-7-fluoroquinazolin-4-amine

The title compound was synthesized using the similar procedure followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 61%; ¹H-NMR-NMR (400 MHz, DMSO-d₆): δ ppm 8.82 (d, J=5.6 Hz, 1H), 7.88 (d, J=8.0 Hz, 1H), 7.40 (d, J=13.6 Hz, 1H), 6.86 (s, 1H), 6.82 (s, 1H), 6.73 (s, 1H), 5.60 (s, 2H), 5.47 (quin, J=7.2 Hz, 1H), 3.13 (bs, 4H), 2.52 (bs, 4H), 2.42 (bs, 2H), 1.57 (d, J=7.2 Hz, 3H), 1.06 (bs, 3H); LCMS: m/z 497.10 [M+H]⁺.

### Example—71: (R)—N-(1-(3-amino-5-(trifluoromethyl)phenyl)ethyl)-2-chloro-6-(4-ethylpiperazin-1-yl)-7-methylquinazolin-4-amine

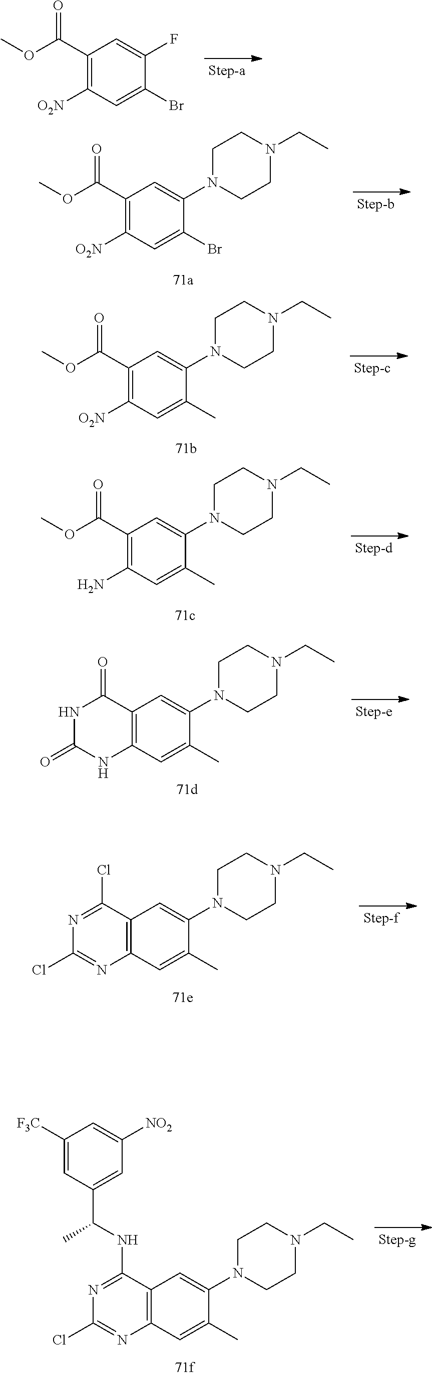

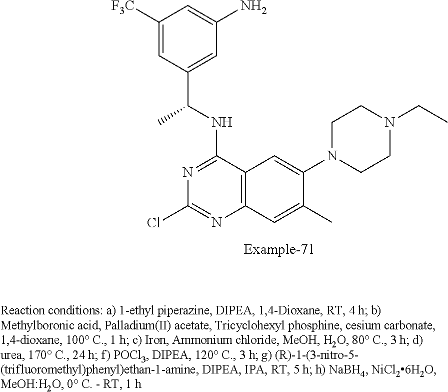 [SMILES: CCN1CCN(c2cc3c(N[C@H](C)c4cc(N)cc(C(F)(F)F)c4)nc(Cl)nc3cc2C)CC1]

### Step—a: ethyl 4-bromo-5-(4-ethylpiperazin-1-yl)-2-nitrobenzoate (71a)

The title compound was synthesized using the similar procedure followed for methyl 5-(4-ethylpiperazin-1-yl)-4-fluoro-2-nitrobenzoate (70a); Yield: 56%; LCMS: m/z 372.0 [M+H]⁺.

### Step—b: ethyl 5-(4-ethylpiperazin-1-yl)-4-methyl-2-nitrobenzoate (71b)

To a solution of ethyl 4-bromo-5-(4-ethylpiperazin-1-yl)-2-nitrobenzoate (500 mg, 1.35 mmol) in 1,4-dioxane (8 mL) was added cesium carbonate (1.32 g, 4.05 mmol), methylboronic acid (162 mg, 2.7 mmol) and tricyclohexylphosphine (76 mg. 0.27 mmol), then the reaction mixture was purged with nitrogen for 2 to 3 minutes. To the reaction mixture Palladium (II) acetate was added and purged with nitrogen for 2 min, then the vial was capped and irradiated in microwave reactor at 100° C. for 1 h. The reaction mixture was diluted with ethyl acetate (50 mL) and filtered through celite pad, filtrate was washed with brine solution (20 mL). The organic layer was dried over anhydrous sodium sulfate and evaporated under reduced pressure. The residue was purified by flash column chromatography using 10% methanol in DCM as an eluent to afford the title compound as a brown solid. Yield: 380 mg (91%); LCMS: m/z 308.10 [M+H]⁺.

### Step—c: ethyl 2-amino-5-(4-ethylpiperazin-1-yl)-4-methylbenzoate (71c)

To a stirred solution of ethyl 5-(4-ethylpiperazin-1-yl)-4-methyl-2-nitrobenzoate (1.0 g, 3.11 mmol) in methanol (20 mL) and water (5 mL) at ambient temperature was added ammonium chloride (1.6 g, 31.1 mmol) and iron powder (868 mg, 15.55 mmol). The reaction mixture was heated at 80° C. for 1 h. Progress of the reaction was monitored by TLC. The reaction mixture was cooled to RT, diluted with ethyl acetate (50 mL), filtered through celite pad. The organic layer was washed saturated NaHCO₃ (20 mL) solution. Aqueous layer was extracted with ethyl acetate (2×30 mL). The combined organic layers were washed with brine solution (40 mL), dried over Na₂SO₄ and concentrated under reduced pressure. The residue was purified by flash column chromatography using 7% MeOH in DCM as an eluent to afford the title compound as a brown solid (Yield: 447 mg, 65%); LCMS: m/z 278.0 [M+H]⁺.

### Step—d: 6-(4-ethylpiperazin-1-yl)-7-methylquinazoline-2,4(1H,3H)-dione (71d)

A mixture of ethyl 2-amino-5-(4-ethylpiperazin-1-yl)-4-methylbenzoate (620 mg, 2.13 mmol) and urea (1.27 g, 21.3 mmol) was heated at 170° C. for 24 h. The reaction mixture was cooled to RT, diluted with water (20 ml) and filtered under vacuum. The solid obtained was suspended in 20% methanol in DCM and stirred for 10 min and filtered under vacuum. The filtrate was concentrated under reduced pressure and to afford the title compound as a pale brown solid which was used in the next step without any further purification (Yield: 325 mg, 53%); LCMS: m/z 289.0 [M+H]⁺.

### Step—e: 2,4-dichloro-6-(4-ethylpiperazin-1-yl)-7-methylquinazoline (71e)

To a solution of 6-(4-ethylpiperazin-1-yl)-7-methylquinazoline-2,4(1H,3H)-dione (275 mg, 0.95 mmol) in phosphorus(V) oxychloride (5.5 mL) was added diisopropylethylamine (0.33 mL, 1.9 mmol) at RT and the reaction mixture was stirred at 120° C. for 3 h. The progress of the reaction was monitored by TLC. Upon completion of the reaction, phosphorus(V)oxychloride was distilled off under reduced pressure. Obtained residue was diluted with DCM (30 mL) and washed with saturated sodium bicarbonate solution (20 mL). Aqueous layer was extracted with DCM (3×20 mL). The combined organic layers were washed with brine solution (30 mL), dried over anhydrous sodium sulfate and concentrated under reduced pressure to afford the title compound. Yield: 100 mg; LCMS: m/z 325.0 [M+H]⁺.

### Step—f: (R)-2-chloro-6-(4-ethylpiperazin-1-yl)-7-methyl-N-(1-(3-nitro-5-(trifluoromethyl)phenyl)ethyl)quinazolin-4-amine (71f)

The title compound was synthesized using the similar procedure followed for R)-2-Chloro-N-(1-(3-nitro-5-(trifluoromethyl)phenyl)ethyl)-6-(pyrrolidin-1-yl)pyrido[3,4-d]pyrimidin-4-amine (If); Yield: 40%; LCMS: m/z 523.10 [M+H]⁺.

### Step—g: (R)—N-(1-(3-amino-5-(trifluoromethyl)phenyl)ethyl)-2-chloro-6-(4-ethylpiperazin-1-yl)-7-methylquinazolin-4-amine

The title compound was synthesized using the similar procedure followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 10%; ¹H-NMR-NMR (400 MHz, DMSO-d₆): δ ppm 8.71 (d, J=7.6 Hz, 1H), 7.91 (s, 1H), 7.44 (s, 1H), 6.86 (s, 1H), 6.82 (s, 1H), 6.72 (s, 1H), 5.59 (s, 2H), 5.46 (quin, J=7.2 Hz, 1H), 2.97 (bs, 4H), 2.50 (bs, 4H), 2.42 (q, J=7.2 Hz, 2H), 2.39 (s, 3H), 1.57 (d, J=7.2 Hz, 3H), 1.05 (t, J=7.2 Hz, 3H); LCMS: m/z 493.10 [M+H]⁺.

### Example—72: (R)—N-(1-(3-amino-5-(trifluoromethyl)phenyl)ethyl)-2-chloro-6-(4-ethylpiperazin-1-yl)-7-methoxyquinazolin-4-amine

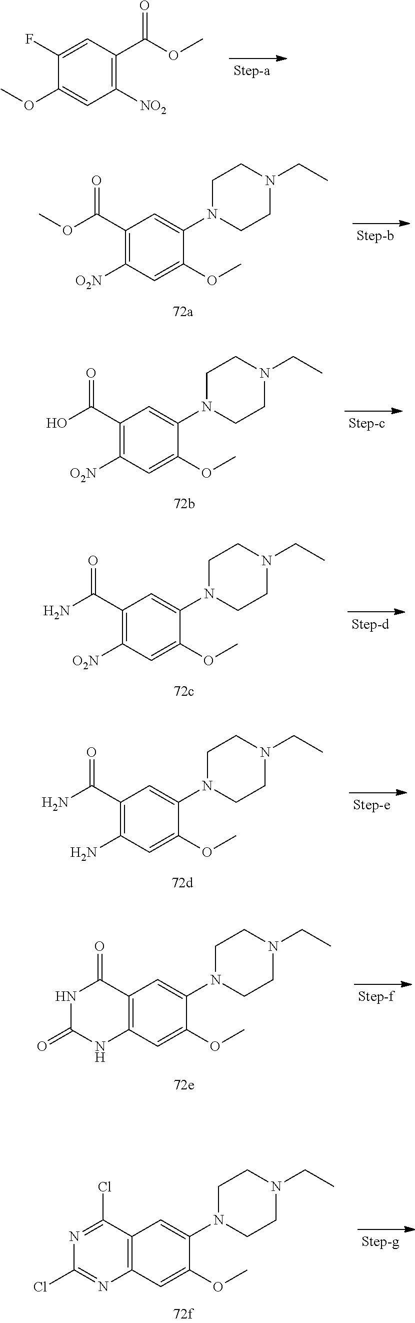

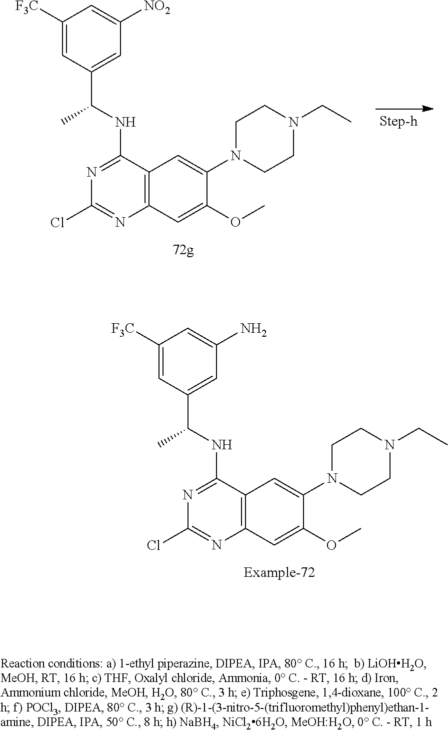 [SMILES: CCN1CCN(c2cc3c(N[C@H](C)c4cc([N+](=O)[O-])cc(C(F)(F)F)c4)nc(Cl)nc3cc2OC)CC1 ; CCN1CCN(c2cc3c(N[C@H](C)c4cc(N)cc(C(F)(F)F)c4)nc(Cl)nc3cc2OC)CC1]

### Step—a: methyl 5-(4-ethylpiperazin-1-yl)-4-methoxy-2-nitrobenzoate (72a)

To a solution of methyl 5-fluoro-4-methoxy-2-nitrobenzoate (2.5 g, 10.92 mmol) in isopropanol (25 mL) was added N,N-diisopropylethylamine (3.79 mL, 21.83 mmol) and 1-ethyl piperazine (2.48 g, 21.83 mmol. The reaction mixture was stirred at 80° C. for 16 h. Progress of the reaction was monitored by TLC. After completion of the reaction isopropanol was distilled off under reduced pressure and the residue was extracted with DCM (100 mL) and water (30 mL), aqueous layer was extracted with DCM (2×100 mL). The combined organic layers were washed with brine solution (30 mL), dried over anhydrous sodium sulphate and concentrated under reduced pressure. The residue was purified by flash column chromatography using 3% MeOH in DCM as n eluent to afford the title compound (Yield: 1.5 g, 43%); LCMS: m/z 324.0 [M+H]⁺.

### Step—b: 5-(4-ethylpiperazin-1-yl)-4-methoxy-2-nitrobenzoic acid (72b)

To a solution of methyl 5-(4-ethylpiperazin-1-yl)-4-methoxy-2-nitrobenzoate (1.0 g, 3.05 mmol) in methanol (10 mL) and THF (10 mL) was added lithium hydroxide monohydrate (384 mg, 9.146 mmol, in 2 mL of water) and the reaction mixture was stirred at RT for 16 h. Progress of the reaction was monitored by TLC. After completion of the reaction, solvent was distilled off under reduced pressure and the residue was extracted diluted with ethyl acetate (100 mL) and neutralized with Amberlite resin and stirred for 15 min. The mixture was filtered and filtrate was concentrated under reduced pressure to afford the title compound (Yield: 1.0 g); LCMS: m/z 310.1 [M+H]⁺.

### Step—c: 5-(4-ethylpiperazin-1-yl)-4-methoxy-2-nitrobenzamide (72c)

To a stirred solution of 5-(4-ethylpiperazin-1-yl)-4-methoxy-2-nitrobenzoic acid (1.0 g, 3.05 mmol) in DCM (15 mL) at 0° C. was added oxalyl chloride (1.1 mL, 12.94 mmol), followed by the addition of DMF (0.1 mL) reaction mixture as stirred for 2 h at RT. The reaction mixture was concentrated under reduced pressure, the residue was dissolved in dry THF (20 mL) and was added to the pre cooled aqueous ammonia at 0° C. in dropwise manner, stirred at RT for 16 h. The reaction mixture was distilled under reduced pressure, the residue was diluted with ethyl acetate (50 mL) and stirred for 5 min. The mixture was filtered and the filtrate was concentrated under reduced pressure to afford the title compound (Yield: 1.0 g, 100%); LCMS: m/z 309.0 [M+H]⁺.

### Step—d: 2-amino-5-(4-ethylpiperazin-1-yl)-4-methoxybenzamide (72d)

The title compound was synthesized using the similar procedure which was followed for ethyl 2-amino-5-(4-ethylpiperazin-1-yl)-4-methylbenzoate (71c); Yield: 75%; LCMS: m/z 279.0 [M+H]⁺.

### Step—e: 6-(4-ethylpiperazin-1-yl)-7-methoxyquinazoline-2,4(1H,3H)-dione (72e)

The title compound was synthesized using the similar procedure which was followed for intermediate 6-(Pyrrolidin-1-yl)pyrido[3,4-d]pyrimidine-2,4(1H,3H)-dione (1d) (Yield: 94%); ¹H-NMR-NMR (400 MHz, DMSO-d₆): δ ppm 11.13 (s, 1H), 11.02 (s, 1H), 7.30 (s, 1H), 6.73 (s, 1H), 3.86 (s, 3H), 3.53 (d, J=11.2 Hz, 2H), 3.45 (d, J=11.2 Hz, 2H), 3.16-3.04 (m, 6H), 1.28 (t, J=7.2 Hz, 3H); LCMS: m/z 305.0 [M+H]⁺.

### Step—f: 2,4-dichloro-6-(4-ethylpiperazin-1-yl)-7-methoxyquinazoline (72f)

The title compound was synthesized using the similar procedure which was followed for intermediate 2,4-dichloro-6-(4-ethylpiperazin-1-yl)-7-methylquinazoline (71e) (Yield: 48%); LCMS: m/z 341.10 [M+H]⁺.

### Step—g: (R)-2-chloro-6-(4-ethylpiperazin-1-yl)-7-methoxy-N-(1-(3-nitro-5-(trifluoromethyl)phenyl)ethyl)quinazolin-4-amine (72g)

To a solution of 2,4-dichloro-6-(4-ethylpiperazin-1-yl)-7-methoxyquinazoline (190 mg, 0.56 mmol) in IPA (4 mL) was added DIPEA (0.3 mL, 1.68 mmol) and (R)-1-(3-nitro-5-(trifluoromethyl)phenyl)ethan-1-amine hydrochloride (151 mg, 0.56 mmol). The reaction mixture was stirred at room temperature for 5 h and at 50° C. for 3 h. The progress of the reaction was monitored by TLC. The reaction mixture was diluted with ethyl acetate (30 mL), water (20 mL) and extracted. Aqueous layer was extracted with EtOAc (2×20 mL). The combined organic layers were washed with brine (20 mL), dried over anhydrous sodium sulfate and concentrated under reduced pressure. The residue was purified by flash column chromatography and eluted with 8% Methanol in DCM to afford the title compound as a pale-yellow solid. Yield 130 mg (43%); LCMS: m/z 539.10 [M+H]⁺.

### Step—h: Synthesis of (R)—N-(1-(3-amino-5-(trifluoromethyl)phenyl)ethyl)-2-chloro-6-(4-ethylpiperazin-1-yl)-7-methoxyquinazolin-4-amine

The title compound was synthesized using the similar procedure followed for (R)—N-(1-(3-amino-5-(trifluoromethyl)phenyl)ethyl)-2-chloro-6-(pyrrolidin-1-yl)pyrido[3,4d]pyrimidin-4-amine (example-1); Yield: 33%; ¹H-NMR-NMR (400 MHz, DMSO-d₆): δ ppm 8.55 (d, J=7.6 Hz, 1H), 7.67 (s, 1H), 7.05 (s, 1H), 6.86 (s, 1H), 6.82 (s, 1H), 6.71 (s, 1H), 5.58 (s, 2H), 5.45 (quin, J=7.2 Hz, 1H), 3.90 (s, 3H), 3.08 (bs, 4H), 2.50 (bs, 4H), 2.41 (q, J=7.2 Hz, 2H), 1.56 (d, J=7.2 Hz, 3H), 1.04 (t, J=7.2 Hz, 3H); LCMS: m/z 509.10 [M+H]⁺.

### Example—73: (R)—N-(1-(3-amino-5-(trifluoromethyl)phenyl)ethyl)-2-chloro-6-(4-ethylpiperazin-1-yl)quinazolin-4-amine

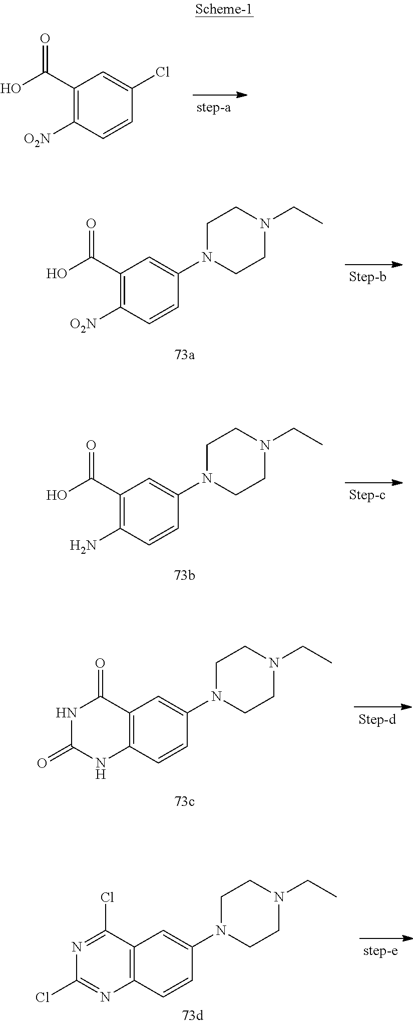 [SMILES: O=C(O)c1cc(Cl)ccc1[N+](=O)[O-] ; CCN1CCN(c2ccc([N+](=O)[O-])c(C(=O)O)c2)CC1 ; CCN1CCN(c2ccc(N)c(C(=O)O)c2)CC1 ; CCN1CCN(c2ccc3[nH]c(=O)[nH]c(=O)c3c2)CC1 ; CCN1CCN(c2ccc3nc(Cl)nc(Cl)c3c2)CC1]

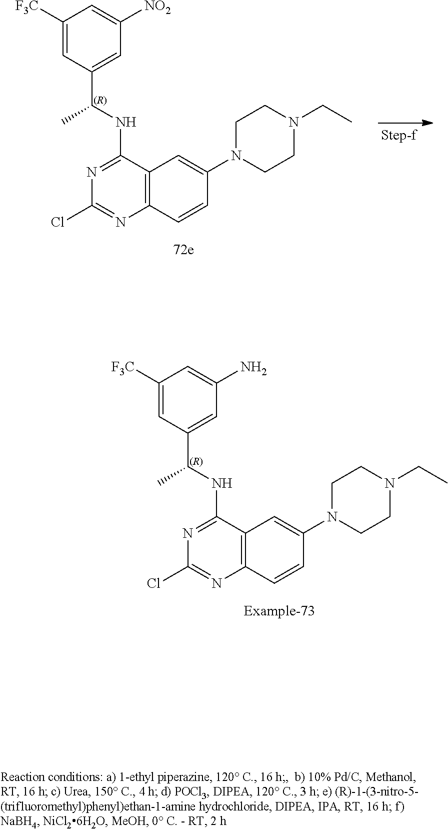

### Step—a: 5-(4-ethylpiperazin-1-yl)-2-nitrobenzoic acid (73a)

A mixture of 5-chloro-2-nitrobenzoic acid (2.0 g, 9.921 mmol) and 1-ethyl piperazine (5.65 g, 49.603 mmol) in a seal tube was heated at 120° C. for 16 h. The reaction mixture was cooled to RT, diluted with cold water (20 mL) and neutralized with iN HCl. The precipitated solid was filtered and dried under vacuum to afford the title compound as a yellow solid. Yield: 2.2 g (81%); LCMS: m/z 280.10 [M+H]⁺.

### Step—b: 2-amino-5-(4-ethylpiperazin-1-yl)benzoic acid (73b)

To a stirred solution of 5-(4-ethylpiperazin-1-yl)-2-nitrobenzoic acid (2.2 g, 7.88 mmol) in methanol (40 mL) was added slurry of 10% Pd—C(50% wet, 300 mg) in methanol (5 mL) and the reaction mixture was stirred under hydrogen atmosphere for 16 h. Reaction progress was monitored by TLC. The reaction mixture was filtered through celite bed, washed with methanol (50 mL) and the filtrate was evaporated under reduced pressure to afford the title compound. Yield: 1.74 g (88%); ¹H-NMR-NMR (400 MHz, DMSO-d₆): δ ppm 10.53 (bs, 1H), 8.41 (bs, 2H), 7.23 (d, J=2.4 Hz, 1H), 7.09 (dd, J=2.4 Hz & 9.2 Hz, 1H), 6.73 (d, J=9.2 Hz, 1H), 3.47 (bs, 4H), 3.17-2.18 (m, 6H), 1.27 (t, J=7.2 Hz, 3H); LCMS: m/z 250.10 [M+H]⁺.

### Step—c: 6-(4-ethylpiperazin-1-yl)quinazoline-2,4(1H,3H)-dione (73c)

A mixture of 2-amino-5-(4-ethylpiperazin-1-yl)benzoic acid (1.74 g, 6.98 mmol) and urea (8.38 g, 139.58 mmol) was heated at 150° C. for 4 h. The reaction mixture was cooled to room temperature and diluted with cold water (30 mL) and the precipitated solid was filtered and dried under vacuum to afford the title compound as a white solid. Yield: 0.85 g (44%); LCMS: m/z 275.20 [M+H]⁺.

### Step—d: 2,4-dichloro-6-(4-ethylpiperazin-1-yl)quinazoline (73d)

The title compound was synthesized using the similar procedure which was followed for intermediate 2,4-dichloro-6-(4-ethylpiperazin-1-yl)-7-methylquinazoline (71e) (Yield: 70%); LCMS: m/z 311.10 [M+H]⁺.

### Step—e: (R)-2-chloro-6-(4-ethylpiperazin-1-yl)-N-(1-(3-nitro-5-(trifluoromethyl)phenyl)ethyl)quinazolin-4-amine (73e)

The title compound was synthesized using the similar procedure which was followed for intermediate (R)-2-chloro-6-(4-ethylpiperazin-1-yl)-7-methyl-N-(1-(3-nitro-5-(trifluoromethyl)phenyl)ethyl)quinazolin-4-amine (71f). (Yield: 46%); LCMS: m/z 509.20 [M+H]⁺.

### Step—f: (R)—N-(1-(3-amino-5-(trifluoromethyl)phenyl)ethyl)-2-chloro-6-(4-ethylpiperazin-1-yl)quinazolin-4-amine

The title compound was synthesized using the similar procedure followed for (R)—N-(1-(3-amino-5-(trifluoromethyl)phenyl)ethyl)-2-chloro-6-(pyrrolidin-1-yl)pyrido[3,4d]pyrimidin-4-amine (example-1). (Yield: 33%); ¹H-NMR-NMR (400 MHz, DMSO-d₆): δ ppm 8.64 (d, J=7.6 Hz, 1H), 7.61 (s, 1H), 7.59 (s, 1H), 7.49 (d, J=8.8 Hz, 1H), 6.86 (s, 1H), 6.82 (s, 1H), 6.72 (s, 1H), 5.59 (s, 2H), 5.47 (quin, J=7.2 Hz, 1H), 3.30 (bs, 4H), 2.55 (bs, 4H), 2.41 (q, J=6.8 Hz, 2H), 1.57 (d, J=7.2 Hz, 3H), 1.06 (t, J=6.8 Hz, 3H); LCMS: m/z 479.10 [M+H]⁺.

### Example—74: (R)—N4-(1-(3-amino-5-(trifluoromethyl)phenyl)ethyl)-N2-methyl-6-morpholinoquinazoline-2,4-diamine

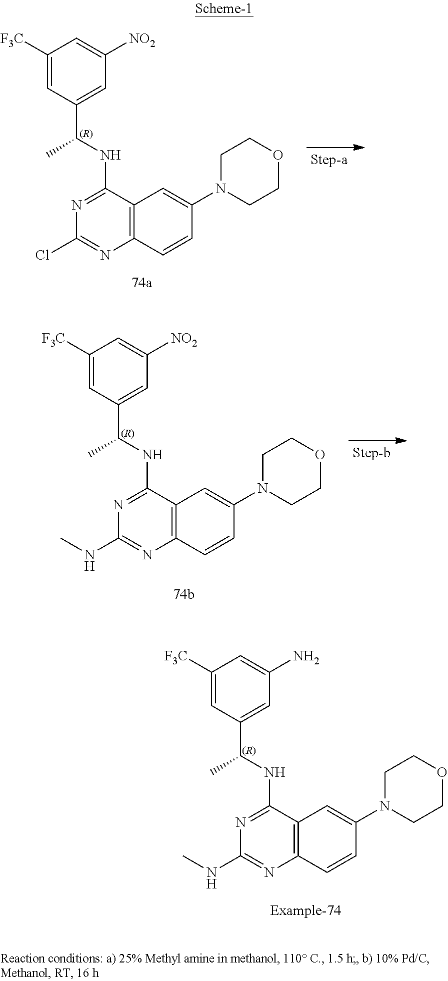

### Synthesis of (R)-2-chloro-6-morpholino-N-(1-(3-nitro-5-(trifluoromethyl)phenyl)ethyl)quinazolin-4-amine (74a)

Intermediate 73a prepared analogous to preparation of (R)-2-chloro-6-(4-ethylpiperazin-1-yl)-N-(1-(3-nitro-5-(trifluoromethyl)phenyl)ethyl)quinazolin-4-amine (72e); LCMS: m/z 482.10 [M+H]⁺.

### Step—a: (R)—N2-methyl-6-morpholino-N4-(1-(3-nitro-5-(trifluoromethyl)phenyl)ethyl)quinazoline-2,4-diamine (74b)

A solution of (R)-2-chloro-6-morpholino-N-(1-(3-nitro-5-(trifluoromethyl)phenyl)ethyl)quinazolin-4-amine (162 mg, 0.336 mmol) in 25% methyl amine in methanol (4 ml) was irradiated in microwave at 110° C. for 1.5 h. The reaction mixture was concentrated under reduced pressure, diluted with water and extracted with ethyl acetate (3×15 mL). The combined organic layer was washed with brine (20 mL), dried over anhydrous sodium sulfate, filtered and concentrated under reduced pressure. The crude compound was purified by flash column chromatography using 7% methanol in DCM as an eluent to afford the title compound. Yield: 114 mg (71%); LCMS: m/z 477.30 [M+H]⁺.

### Step—b: (R)—N4-(1-(3-amino-5-(trifluoromethyl)phenyl)ethyl)-N2-methyl-6-morpholinoquinazoline-2,4-diamine

The title compound was synthesized using the similar procedure followed for (R)—N4-(1-(3-amino-5-(trifluoromethyl)phenyl)ethyl)-N2,N2-dimethyl-6-(tetrahydro-2H-pyran-4-yl)pyrido[3,4-d]pyrimidine-2,4-diamine (example—65). (Yield: 22%); ¹H-NMR-NMR (400 MHz, DMSO-d₆): δ ppm 7.95 (bs, 1H), 7.49 (d, J=2.4 Hz, 1H), 7.32 (dd, J=2.4 Hz & 9.2 Hz, 1H), 7.19 (d, J=9.2 Hz, 1H), 6.83 (s, 2H), 6.69 (s, 1H), 6.13 (bs, 1H), 5.52 (s, 2H), 5.47 (t, J=6.8 Hz, 1H), 3.78 (d, J=4.8 Hz, 4H), 3.13 (bs, 4H), 2.74 (d, J=4.8 Hz, 3H), 1.53 (d, J=7.2 Hz, 3H); LCMS: m/z 447.20 [M+H]⁺.

### Example—75: (R)—N-(4-((1-(3-amino-5-(trifluoromethyl)phenyl)ethyl)amino)-6-(pyrrolidin-1-yl)pyrido[3,4-d]pyrimidin-2-yl)propionamide

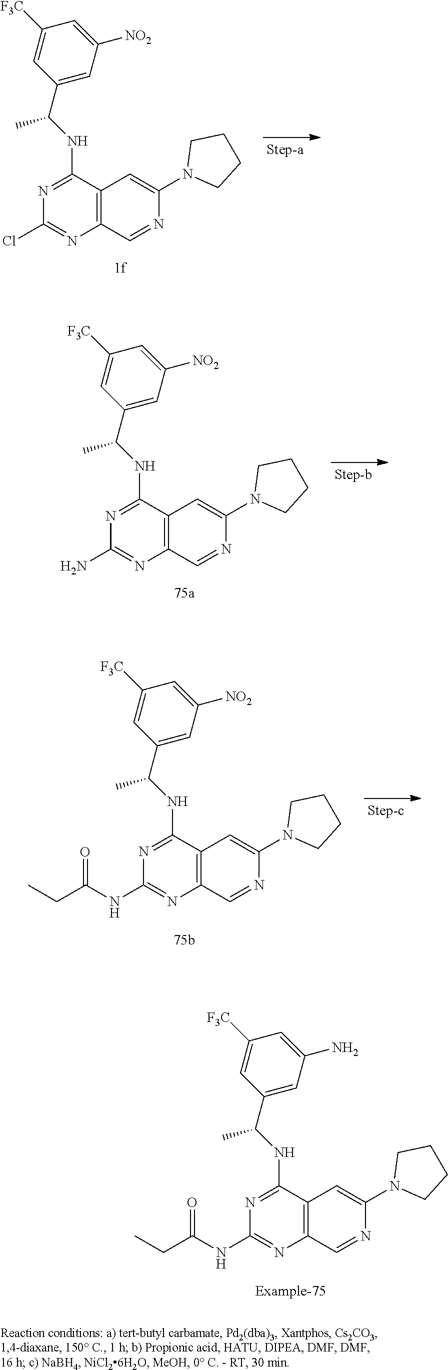 [SMILES: C[C@@H](Nc1nc(Cl)nc2cnc(N3CCCC3)cc12)c1cc([N+](=O)[O-])cc(C(F)(F)F)c1 ; C[C@@H](Nc1nc(N)nc2cnc(N3CCCC3)cc12)c1cc([N+](=O)[O-])cc(C(F)(F)F)c1 ; CCC(=O)Nc1nc(N[C@H](C)c2cc([N+](=O)[O-])cc(C(F)(F)F)c2)c2cc(N3CCCC3)ncc2n1 ; CCC(=O)Nc1nc(N[C@H](C)c2cc(N)cc(C(F)(F)F)c2)c2cc(N3CCCC3)ncc2n1]

### Step—a: (R)—N4-(1-(3-nitro-5-(trifluoromethyl)phenyl)ethyl)-6-(pyrrolidin-1-yl)pyrido[3,4-d]pyrimidine-2,4-diamine (75a)

To a solution of (R)-2-chloro-N-(1-(3-nitro-5-(trifluoromethyl)phenyl)ethyl)-6-(pyrrolidin-1-yl)pyrido[3,4-d]pyrimidin-4-amine (300 mg, 0.64 mmol) in 1,4-dioxane (4 mL) were added tert-butyl carbamate (376 mg, 3.21 mmol), xantphos (55 mg, 0.096 mmol), cesium carbonate (627 mg, 1.92 mmol) and Pd₂(dba)₃ (14 mg. 0.064 mmol). The mixture was purged with nitrogen for 5 min, the microwave vial was capped and irradiated in microwave reactor at 150° C. for 1 h. The reaction mixture was diluted with EtOAc (20 mL) and water (20 mL) then extracted, aqueous layer was extracted with ethyl acetate (2×15 mL). The combined organic extracts were washed with brine (15 mL), dried over anhydrous sodium sulfate and evaporated under reduced pressure. The residue was purified by flash column chromatography using 10% MeOH in DCM as an eluent to afford R)—N4-(1-(3-nitro-5-(trifluoromethyl)phenyl)ethyl)-6-(pyrrolidin-1-yl)pyrido[3,4-d]pyrimidine-2,4-diamine as a brown solid. Yield: 220 mg (77%); LCMS: m/z 448.10 [M+H]⁺.

### Step—b: (R)—N-(4-((1-(3-nitro-5-(trifluoromethyl)phenyl)ethyl)amino)-6-(pyrrolidin-1-yl)pyrido[3,4-d]pyrimidin-2-yl)propionamide (75b)

To a stirred solution of (R)—N4-(1-(3-nitro-5-(trifluoromethyl)phenyl)ethyl)-6-(pyrrolidin-1-yl)pyrido[3,4-d]pyrimidine-2,4-diamine (220 mg, 0.49 mmol) in DMF (5 mL) at RT were added DIPEA (0.7 mL, 3.92 mmol), HATU (279 mg, 0.735 mmol) and Propionic acid (0.07 mL, 0.98 mmol), then the reaction mixture was stirred at RT for 16 h. Progress of the reaction was monitored by TLC. The reaction mixture was quenched with water (15 mL) and extracted with ethyl acetate (3×20 mL). The combined organic extracts were washed with brine (30 mL), dried over anhydrous sodium sulfate and evaporated under reduced pressure. The residue was purified by flash column chromatography using 10% MeOH in DCM as an eluent to afford the title compound as a pale brown solid. Yield: 60 mg (24%); LCMS: m/z 504.20 [M+H]⁺.

### Step—c: (R)—N-(4-((1-(3-amino-5-(trifluoromethyl)phenyl)ethyl)amino)-6-(pyrrolidin-1-yl)pyrido[3,4-d]pyrimidin-2-yl)propionamide

The title compound was synthesized using the similar procedure followed for (R)—N-(1-(3-amino-5-(trifluoromethyl)phenyl)ethyl)-2-chloro-6-(pyrrolidin-1-yl)pyrido[3,4d]pyrimidin-4-amine (example-1). (Yield: 44%); ¹H-NMR-NMR (400 MHz, DMSO-d₆): δ ppm 9.65 (s, 1H), 8.54 (s, 1H), 8.46 (d, J=8.0 Hz, 1H), 7.07 (s, 1H), 6.87 (s, 1H), 6.84 (s, 1H), 6.71 (s, 1H), 5.58-5.52 (m, 3H), 3.46 (bs, 4H), 2.50 (2H merged with DMSO solvent peak), 2.0 (t, J=6.4 Hz, 4H), 1.57 (d, J=7.2 Hz, 3H), 0.99 (t, J=7.6 Hz, 3H); LCMS: m/z 474.20 [M+H]⁺.

TABLE 8
Examples in table-8 were prepared by using process analogous to the preparation
of example-75

| | | | 1H NMR (400 MHz, DMSO- |
|---|---|---|---|
| | | | d₆): δppm |
| Example # | Structure | IUPAC Name | LC-MS: m/z |
| 76 | 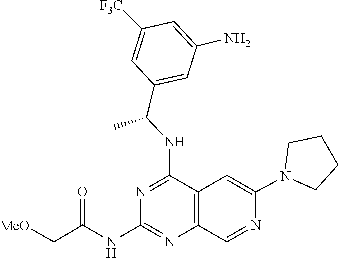 [SMILES: COCC(=O)Nc1nc(N[C@H](C)c2cc(N)cc(C(F)(F)F)c2)c2cc(N3CCCC3)ncc2n1] | (R)-N-(4-((1-(3-amino-5- (trifluoromethyl)phenyl) ethyl)amino)-6- (pyrrolidin-1- yl)pyrido[3,4- d]pyrimidin-2-yl)-2- methoxyacetamide | 9.54 (s, 1H), 8.56 (s, 1H), 8.51 (d, J = 8.0 Hz, 1H), 7.08 (s, 1H), 6.87 (s, 1H), 6.84 (s, 1H), 6.71 (s, 1H), 5.54-5.49 (m, 3H), 4.27-4.12 (m, 2H), 3.46 (bs, 4H), 3.30 (s, 3H), 2.01 (t, J = 6.4 Hz, 4H), 1.58 (d, J = 7.2 Hz, 3H) 490.10 [M + H]⁺ |
| 77 | 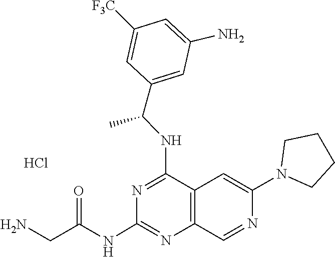 [SMILES: C[C@@H](Nc1nc(NC(=O)CN)nc2cnc(N3CCCC3)cc12)c1cc(N)cc(C(F)(F)F)c1 ; Cl] | (R)-2-amino-N-(4-((1-(3- amino-5- (trifluoromethyl)phenyl) ethyl)amino)-6- (pyrrolidin-1- yl)pyrido[3,4- d]pyrimidin-2- yl)acetamide Hydro chloride | 12.20 (bs, 1H), 10.2 (bs, 1H), 9.05 (s, 1H), 8.43 (bs, 3H), 7.43 (s, 1H), 7.09 (s, 1H), 7.04 (s, 1H), 6.87 (s, 1H), 5.68 (quin, J = 7.2 Hz, 1H), 4.60 (bs, 2H), 4.05 (d, J = 4.4 Hz, 2H), 3.48 (d, J = 3.6 Hz, 4H), 2.01 (t, J = 6.4 Hz, 4H), 1.68 (d, J = 6.8 Hz, 3H) 475.20 [M + H]⁺ |
| 78 | 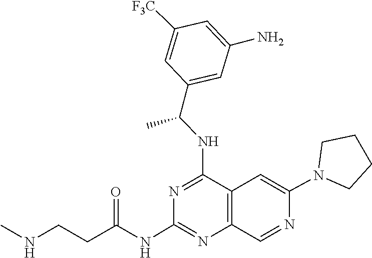 [SMILES: CNCCC(=O)Nc1nc(N[C@H](C)c2cc(N)cc(C(F)(F)F)c2)c2cc(N3CCCC3)ncc2n1] | (R)-N-(4-((1-(3-amino-5- (trifluoromethyl)phenyl) ethyl)amino)-6- (pyrrolidin-1- yl)pyrido[3,4- d]pyrimidin-2-yl)-3- (methylamino) propenamide | 10.03 (bs, 1H), 8.54 (s, 1H), 8.45 (d, J = 8.4 Hz, 1H), 7.06 (s, 1H), 6.88 (s, 1H), 6.86 (s, 1H), 6.71 (s, 1H), 5.54-5.52 (m, 3H), 3.46 (bs, 4H), 2.70 (t, J = 6.4 Hz, 2H), 2.65- 2.55 (m, 2H), 2.28 (s, 3H), 2.0 (bs, 4H), 1.57 (d, J = 6.8 Hz, 3H) (One exchangeable proton not observed in ¹H- NMR) 503.50 [M + H]⁺ |
| 79 | 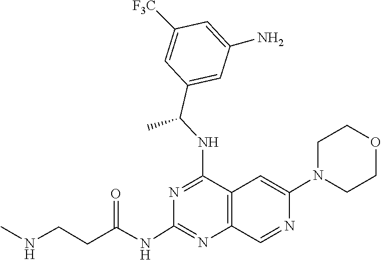 [SMILES: CNCCC(=O)Nc1nc(N[C@H](C)c2cc(N)cc(C(F)(F)F)c2)c2cc(N3CCOCC3)ncc2n1] | (R)-N-(4-((1-(3-amino-5- (trifluoromethyl)phenyl) ethyl)amino)-6- morpholinopyrido[3,4- d]pyrimidin-2-yl)-3- (methylamino) propanamide | 10.19 (s, 1H), 8.61 (s, 1H), 8.59 (d, J = 8.4 Hz, 1H), 7.46 (s, 1H), 6.87 (s, 1H), 6.85 (s, 1H), 6.72 (s, 1H), 5.10-5.02 (m, 3H), 3.77 (t, J = 5.2 Hz, 4H), 3.49 (t, J = 4.4 Hz, 4H), 3.07 (t, J = 6.4 Hz, 2H), 2.92- 2.88 (m, 2H), 2.52 (s, 3H), 1.57 (d, J = 7.2 Hz, 3H) (One exchangeable proton not observed in ¹H-NMR) 519.40 [M + H]⁺ |

### Example—80: Synthesis of (R)—N-(1-(3-amino-5-(trifluoromethyl)phenyl)ethyl)-2-chloro-6-(4-ethylpiperazin-1-yl)pyrido[2,3-d]pyrimidin-4-amine

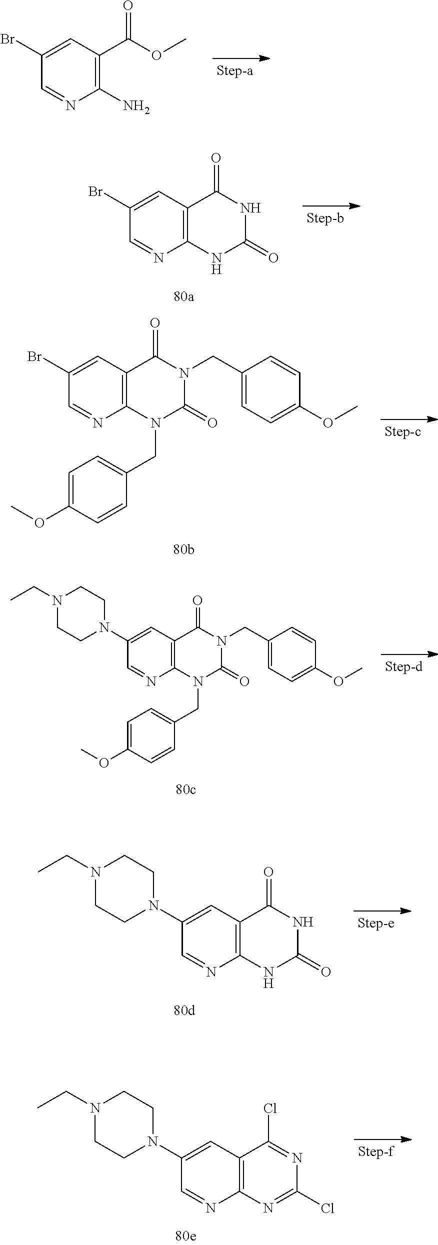

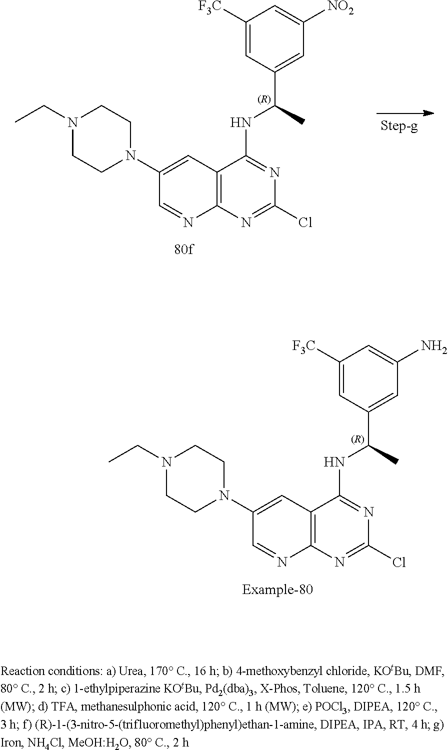

### Step—a: 6-bromopyrido[2,3-d]pyrimidine-2,4(1H,3H)-dione (80a)

A mixture of methyl 2-amino−-bromonicotinate (9.0 g, 38.96 mmol) and urea (670 mg, 3.8 mmol) was stirred at 170 LC for 16 h. The reaction mixture was cooled to room temperature and diluted with water (200 mL) and stirred for 10 m, the precipitated solid was filtered, washed with ethyl acetate (100 mL) and dried under vacuum to afford 6-bromopyrido[j2,3-d]pyrimidine-2,4(1H,3H)-dione as a brown solid. Yield: 12.5 g; LCMS: m/z 241.80 [M+H]⁺.

### Step—b: 6-bromo-1,3-bis(4-methoxybenzyl)pyrido[2,3-d]pyrimidine-2,4(1 H,3H)-dione (80b)

To a solution of 6-bromopyrido[2,3-d]pyrimidine-2,4(1H,3H)-dione (5.0 g, 20.8 mmol) in DMF (60 mL) at 0° C. were added potassium tert-butoxide (5.83 g, 52.08 mmol) and 4-methoxybenzyl chloride (7.0 mL, 52 mmol), then the reaction mixture was stirred at 80° C. for 2 h. The progress of the reaction was monitored by TLC. The reaction mixture was diluted with water (200 mL) and extracted with ethyl acetate (3×100 mL). The combined organic extracts were washed with water (50 mL), brine (50 mL), dried over anhydrous sodium sulfate and evaporated under reduced pressure. The residue obtained was stirred in diethyl ether (50 mL) and the precipitated solid was filtered and dried under vacuum to afford 6-bromo-1,3-bis(4-methoxybenzyl)pyrido[2,3-d]pyrimidine-2,4(1H,3H)-dione as a white solid. Yield: 2.7 g (27%); ¹H-NMR (400 MHz, DMSO-d₆): δ 8.86 (d, J=2.4 Hz, 1H), 8.52 (d, J=2.4 Hz, 1H), 7.31 (t, J=8.4 Hz, 4H), 6.87 (t, J=8.8 Hz, 4H), 5.33 (s, 2H), 5.0 (s, 2H), 3.71 (s, 3H), 3.70 (s, 3H); LCMS: m/z 482.90 [M+H]⁺.

### Step—c: 6-(4-ethylpiperazin-1-yl)-1,3-bis(4-methoxybenzyl)pyrido[2,3-d]pyrimidine-2,4(1H,3H)-dione (80c)

To a solution of 6-bromo-1,3-bis(4-methoxybenzyl)pyrido[2,3-d]pyrimidine-2,4(1H,3H)-dione (2.2 g, 4.58 mmol) in Toluene (20 mL) were added 1-ethylpiperazine (1.16 mL, 9.16 mmol), X-Phos (381 mg, 0.916 mmol) and cesium carbonate (4.4 g, 13.74 mmol) and the mixture was purged with nitrogen gas for 5 min. To the reaction mixture Pd₂(dba)₃ (419 mg. 0.458 mmol) was added and the microwave vial was capped and irradiated in microwave reactor at 120° C. for 1.5 h. The reaction mixture was diluted with EtOAc (50 mL) and filtered through a celite pad, the filtrate was diluted with water (50 mL) and extracted with EtOAc (3×100 mL). The combined organic extracts were washed with brine (50 mL), dried over anhydrous sodium sulfate and evaporated under reduced pressure. The residue was purified by flash column chromatography using 5% MeOH in DCM as an eluent to afford 6-(4-ethylpiperazin-1-yl)-1,3-bis(4-methoxybenzyl)pyrido[2,3-d]pyrimidine-2,4(1H,3H)-dione as a brown solid. Yield: 1.5 g (63%); ¹H-NMR (400 MHz, DMSO-d₆): δ 8.57 (d, J=3.2 Hz, 1H), 7.79 (d, J=2.8 Hz, 1H), 7.29-7.24 (m, 4H), 6.86 (t, J=8.4 Hz, 4H), 5.34 (s, 2H), 5.06 (s, 2H), 3.70 (s, 3H), 3.68 (s, 3H), 3.20 (bs, 4H), 2.50 (bs, 4H), 2.38 (q, J=7.2 Hz, 2H), 1.04 (t, J=7.2 Hz, 3H); LCMS: m/z 516.20 [M+H]⁺.

### Step—d: 6-(4-ethylpiperazin-1-yl)pyrido[2,3-d]pyrimidine-2,4(1H,3H)-dione (80d)

To a solution of 6-(4-ethylpiperazin-1-yl)-1,3-bis(4-methoxybenzyl)pyrido[2,3-d]pyrimidine-2,4(1H,3H)-dione (1.8 g, 3.48 mmol) in trifluoroacetic acid (8 mL mL) was added methanesulphonic acid (8 mL) and the microwave vial was capped and irradiated in microwave reactor at 120° C. for 1 h. The reaction mixture was evaporated under reduced pressure and the residue was basified to pH˜ 6 with saturated sodium bicarbonate solution and extracted with DCM (3×300 mL). The combined organic extracts were washed with brine (50 mL), dried over anhydrous sodium sulfate and evaporated under reduced pressure. The residue was purified by flash column chromatography using 10% MeOH in DCM as an eluent to afford 6-(4-ethylpiperazin-1-yl)pyrido[2,3-d]pyrimidine-2,4(1H,3H)-dione as a brown solid. Yield: 800 mg (46%); ¹H-NMR (400 MHz, DMSO-d₆): δ 11.48 (s, 1H), 11.36 (s, 1H), 8.46 (d, J=2.8 Hz, 1H), 7.63 (s, 1H), 3.17 (bs, 4H), 2.56 (bs, 4H), 2.37 (bs, 2H), 1.05 (t, J=6.0 Hz, 3H); LCMS: m/z 276.0 [M+H]⁺.

### Step—e: 2,4-dichloro-6-(4-ethylpiperazin-1-yl)pyrido[2,3-d]pyrimidine (80e)

To a solution of 6-(4-ethylpiperazin-1-yl)pyrido[2,3-d]pyrimidine-2,4(1H,3H)-dione (400 mg, 1.45 mmol) in phosphorus(V) oxychloride (4 mL) was added diisopropylethylamine (0.52 mL, 2.90 mmol) at RT and the reaction mixture was stirred at 120° C. for 3 h. The progress of the reaction was monitored by TLC. Phosphorus(V)oxychloride was distilled off under reduced pressure. Obtained residue was dissolved in DCM (50 mL) and basified with saturated sodium bicarbonate solution then extracted with DCM (3×100 mL). The combined organic extracts were washed with brine (50 mL), dried over anhydrous sodium sulfate and concentrated under reduced pressure to afford 2,4-dichloro-6-(4-ethylpiperazin-1-yl)pyrido[2,3-d]pyrimidine as a pale red solid. Crude compound was used in the following step without further purification. Yield: 300 mg (66%); LCMS: m/z 312.0 [M+H]⁺.

### Step—f: (R)-2-chloro-6-(4-ethylpiperazin-1-yl)-N-(1-(3-nitro-5-(trifluoromethyl)phenyl)ethyl)pyrido[2,3-d]pyrimidin-4-amine (80f)

To a solution of 2,4-dichloro-6-(4-ethylpiperazin-1-yl)pyrido[2,3-d]pyrimidine (300 mg, 0.964 mmol) in IPA (5 mL) were added DIPEA (0.53 mL, 2.89 mmol) and (R)-1-(3-nitro-5-(trifluoromethyl)phenyl)ethan-1-amine hydrochloride (260 mg, 0.964 mmol). The reaction mixture was stirred at room temperature for 4 h. Reaction progress was monitored by TLC. The reaction mixture was quenched with water (20 mL), extracted with EtOAc (3×40 mL). The combined organic extracts were washed with brine (30 mL), dried over anhydrous sodium sulfate and concentrated under reduced pressure. The residue obtained was purified by flash column chromatography and eluted with 2% mthanol in DCM to afford (R)-2-chloro-6-(4-ethylpiperazin-1-yl)-N-(1-(3-nitro-5-(trifluoromethyl)phenyl) ethyl)pyrido[2,3-d]pyrimidin-4-amine as yellow solid. Yield 120 mg (24%); LCMS: m/z 510.0 [M+H]⁺.

### Step—g: (R)—N-(1-(3-amino-5-(trifluoromethyl)phenyl)ethyl)-2-chloro-6-(4-ethylpiperazin-1-yl)pyrido[2,3-d]pyrimidin-4-amine

To a stirred solution of (R)-2-chloro-6-(4-ethylpiperazin-1-yl)-N-(1-(3-nitro-5-(trifluoromethyl)phenyl)ethyl)pyrido[2,3-d]pyrimidin-4-amine (120 mg, 0.235 mmol) in methanol (2.4 mL) and water (0.6 mL) at ambient temperature were added iron powder (65 mg, 1.175 mmol) and ammonium chloride (125 mg, 2.35 mmol). The reaction mixture was heated at 80° C. for 2 h. The progress of the reaction was monitored by TLC. The reaction mixture was diluted with ethyl acetate (50 mL), filtered through celite bed. The organic layer was washed with water (30 mL), the aqueous layer was extracted with EtOAc (2×30 mL). The combined organic extracts were washed with saturated NaHCO₃ (20 mL) solution, dried over Na₂SO₄ and concentrated under reduced pressure. The residue was purified by flash column chromatography using 6% MeOH in DCM as an eluent to afford (R)—N-(1-(3-amino-5-(trifluoromethyl)phenyl)ethyl)-2-chloro-6-(4-ethylpiperazin-1-yl)pyrido[2,3-d]pyrimidin-4-amine as a yellow solid. Yield: 30 mg (27%). ¹H-NMR (400 MHz, DMSO-d₆): δ 8.90 (d, J=2.8 Hz, 1H), 8.88 (d, J=8.4 Hz, 1H), 8.06 (d, J=2.8 Hz, 1H), 6.85 (s, 1H), 6.81 (s, 1H), 6.73 (s, 1H), 5.60 (bs, 2H), 5.47-5.43 (m, 1H), 3.30 (bs, 4H), 2.56 (bs, 4H), 2.41 (q, J=6.4 Hz, 2H), 1.58 (d, J=7.2 Hz, 3H), 1.07 (t, J=7.2 Hz, 3H); LCMS: m/z 479.90 [M+H]⁺.

### Example—81: (R)—N-(1-(3-amino-5-(trifluoromethyl)phenyl)ethyl)-6-(4-ethylpiperazin-1-yl)-2-methoxypyrido[2,3-d]pyrimidin-4-amine

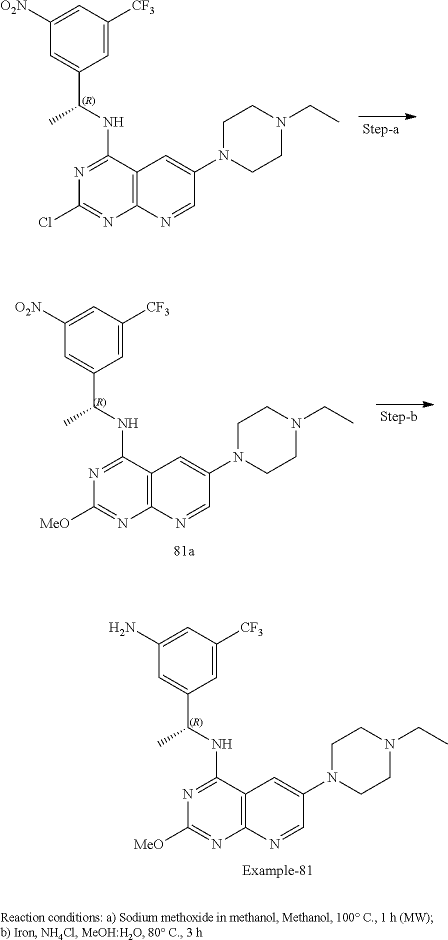

### Step—a: (R)-6-(4-ethylpiperazin-1-yl)-2-methoxy-N-(1-(3-nitro-5-(trifluoromethyl) phenyl)ethyl)pyrido[2,3-d]pyrimidin-4-amine (81a)

To a solution of (R)-2-chloro-6-(4-ethylpiperazin-1-yl)-N-(1-(3-nitro-5-(trifluoromethyl)phenyl)ethyl)pyrido[2,3-d]pyrimidin-4-amine (150 mg, 0.29 mmol) in methanol (5 mL mL) was added 25% sodium methoxide in methanol (2 mL) and the microwave vial was capped and irradiated in microwave reactor at 100° C. for 1 h. Progress of the reaction was monitored by TLC. The reaction mixture was quenched with water (20 mL) and extracted with EtOAc (3×50 mL). The combined organic extracts were washed with brine (20 mL), dried over anhydrous sodium sulfate and evaporated under reduced pressure. The residue was purified by flash column chromatography using 3% MeOH in DCM as an eluent to afford (R)-6-(4-ethylpiperazin-1-yl)-2-methoxy-N-(1-(3-nitro-5-(trifluoromethyl)phenyl)ethyl)pyrido[2,3-d]pyrimidin-4-amine as a yellow solid. Yield: 90 mg (60%). LCMS: m/z 506.10 [M+H]⁺.

### Step—b: (R)—N-(1-(3-amino-5-(trifluoromethyl)phenyl)ethyl)-6-(4-ethylpiperazin-1-yl)-2-methoxypyrido[2,3-d]pyrimidin-4-amine

To a stirred solution of (R)-6-(4-ethylpiperazin-1-yl)-2-methoxy-N-(1-(3-nitro-5-(trifluoromethyl)phenyl)ethyl)pyrido[2,3-d]pyrimidin-4-amine (90 mg, 0.178 mmol) in methanol (2 mL) and water (0.5 mL) at ambient temperature were added iron powder (49 mg, 0.891 mmol) and ammonium chloride (95 mg, 1.78 mmol). The reaction mixture was heated at 80° C. for 3 h. The progress of the reaction was monitored by TLC. The reaction mixture was diluted with ethyl acetate (30 mL), filtered through celite pad. The organic layer was washed with saturated NaHCO₃ (20 mL) solution, the aqueous layer was extracted with EtOAc (2×30 mL). The combined organic extracts were washed with brine (20 mL), dried over Na₂SO₄ and concentrated under reduced pressure. The residue obtained was purified by flash column chromatography using 5% MeOH in DCM as an eluent to afford (R)—N-(1-(3-amino-5-(trifluoromethyl)phenyl)ethyl)-6-(4-ethylpiperazin-1-yl)-2-methoxypyrido[2,3-d]pyrimidin-4-amine as a yellow solid. Yield: 5 mg (6%). ¹H-NMR (400 MHz, DMSO-d₆): δ 8.75 (d, J=2.4 Hz, 1H), 8.53 (d, J=7.2 Hz, 1H), 8.07 (d, J=2.4 Hz, 1H), 6.83 (d, J=10.4 Hz, 2H), 6.70 (s, 1H), 5.56 (bs, 2H), 5.43-5.42 (m, 1H), 3.80 (s, 3H), 3.27 (bs, 4H), 2.56 (bs, 4H), 2.42 (q, J=7.2 Hz, 2H), 1.55 (d, J=7.2 Hz, 3H), 1.07 (t, J=7.2 Hz, 3H); LCMS: m/z 476.0 [M+H]⁺.

TABLE 9
Compounds in table-9 were prepared by using process analogous to the
preparation of example-80

| | | | 1H NMR (400 MHz, |
|---|---|---|---|
| | | | DMSO-d₆):δppm |
| Example # | Structure | IUPAC Name | LC-MS: m/z |
| 82 | 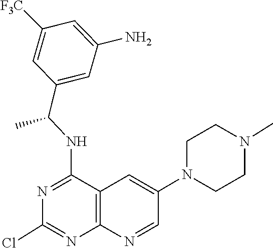 [SMILES: C[C@@H](Nc1nc(Cl)nc2ncc(N3CCN(C)CC3)cc12)c1cc(N)cc(C(F)(F)F)c1] | (R)-N-(1-(3-amino-5- (trifluoromethyl)phenyl) ethyl)-2-chloro-6-(4- methylpiperazin-1- yl)pyrido[2,3- d]pyrimidin-4-amine | 8.90 (d, J = 2.8 Hz, 1H), 8.88 (d, J = 7.2 Hz, 1H), 8.05 (d, J = 2.8 Hz, 1H), 6.85 (s, 1H), 6.81 (s, 1H), 6.72 (s, 1H), 5.59 (bs, 2H), 5.46-5.42 (m, 1H), 3.35 (bs, 4H), 2.51 (bs, 4H), 2.25 (s, 3H), 1.57 (d, J = 7.2 Hz, 3H) 466.10 [M + H]⁺ |
| 83 | 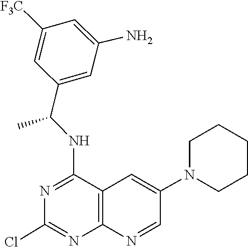 [SMILES: C[C@@H](Nc1nc(Cl)nc2ncc(N3CCCCC3)cc12)c1cc(N)cc(C(F)(F)F)c1] | (R)-N-(1-(3-amino-5- (trifluoromethyl)phenyl) ethyl)-2-chloro-6- (piperidin-1- yl)pyrido[2,3- d]pyrimidin-4-amine | 8.88 (d, J = 2.8 Hz, 1H), 8.85 (s, 1H), 8.03 (d, J = 2.8 Hz, 1H), 6.85 (s, 1H), 6.81 (s, 1H), 6.72 (s, 1H), 5.59 (bs, 2H), 5.44-5.43 (m, 1H), 3.36 (bs, 4H), 1.68 (bs, 4H), 1.60 (d, J = 4.4 Hz, 2H), 1.57 (d, J = 7.2 Hz, 3H) 451.1 [M + H]⁺ |
| 84 | 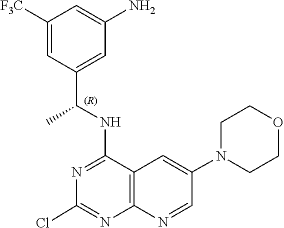 | (R)-N-(1-(3-amino-5- (trifluoromethyl)phenyl) ethyl)-2-chloro-6- morpholinopyrido[2,3- d]pyrimidin-4-amine | 8.90 (d, J = 2.8 Hz, 1H), 8.87 (s, 1H), 8.05 (d, J = 2.8 Hz, 1H), 6.83 (s, 1H), 6.79 (s, 1H), 6.71 (s, 1H), 5.58 (bs, 2H), 5.43-5.42 (m, 1H), 3.80 (t, J = 4.8 Hz, 4H), 3.32 (4H merged with DMSO moisture peak), 1.55 (d, J = 7.2 Hz, 3H) 453.10 [M + H]⁺ |

### Example—85: (R)—N-(1-(3-amino-5-(trifluoromethyl)phenyl)ethyl)-6-(4-ethylpiperazin -1-yl)pyrido[3,4-c]pyridazin-4-amine

### Step—a: (E)-2-(2-chloro-5-nitropyridin-4-yl)-N,N-dimethylethen-1-amine (85a)

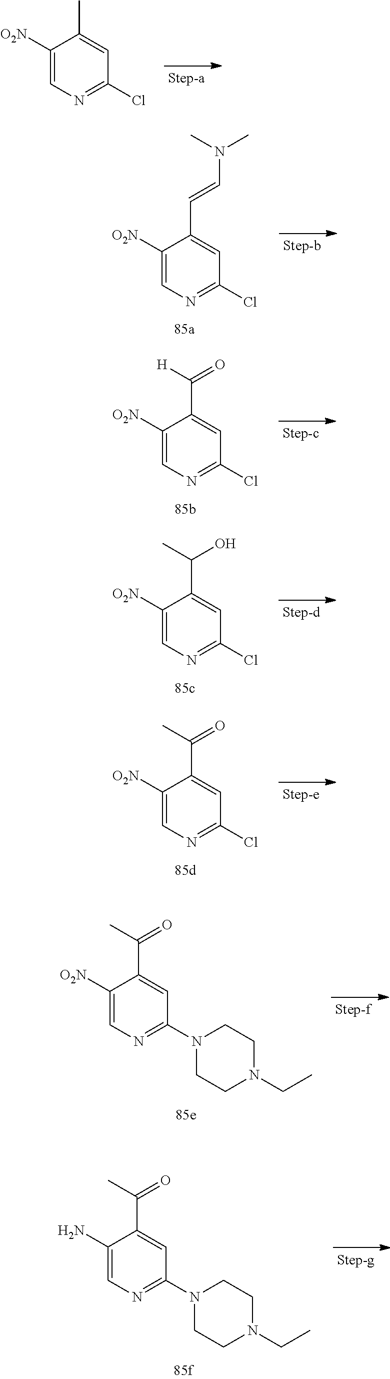

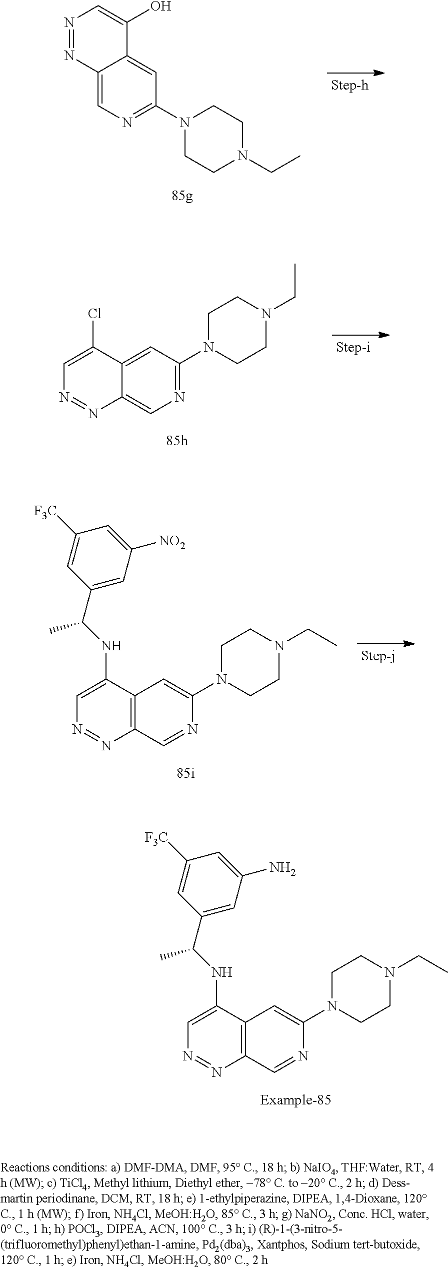

To a solution of 2-chloro-4-methyl-5-nitropyridine (5.0 g, 29.069 mmol) in DMF (25 mL) was added N,N-Dimethylformamide dimethyl acetal (7.61 g, 63.95 mmol) and the reaction mixture was heated at 95° C. for 16 h. Progress of the reaction was monitored by TLC. The reaction mixture was diluted with water (100 mL) and extracted with EtOAc (3×200 mL). The combined organic extracts were washed with brine (50 mL), dried over anhydrous sodium sulfate and evaporated under reduced pressure. The residue was stirred in methanol (30 mL), precipitated solid was filtered and dried under vacuum to afford (E)-2-(2-chloro-5-nitropyridin-4-yl)-N,N-dimethylethen-1-amine. Yield: 3.3 g (51%); ¹H-NMR (400 MHz, CDCl₃): δ 8.79 (s, 1H), 7.33 (d, J=13.2 Hz, 1H), 7.25 (s, 1H), 5.97 (d, J=13.2 Hz, 1H), 3.06 (bs, 6H).

### Step—b: 2-chloro-5-nitroisonicotinaldehyde (85b)

To a solution of (E)-2-(2-chloro-5-nitropyridin-4-yl)-N,N-dimethylethen-1-amine (2 g, 8.81 mmol) in 1:1 mixture of THF and water (40 mL) was added Sodium periodate (5.65 g, 26.43 mmol) and the contents of the reaction was stirred at ambient temperature for 6 h. The progress of the reaction was monitored by TLC. The solid was filtered off and the filtrate was diluted with water (30 mL), extracted with EtOAc (3×100 mL). The combined organic extracts were dried over anhydrous sodium sulfate and evaporated under reduced pressure. The residue was purified by flash column chromatography using 10% EtOAc in hexane as an eluent to afford 2-chloro-5-nitroisonicotinaldehyde. Yield: 1.0 g (61%); ¹H-NMR (400 MHz, CDCl₃): δ 10.51 (s, 1H), 9.25 (s, 1H), 7.70 (s, 1H).

### Step—c: Synthesis of 1-(2-chloro-5-nitropyridin-4-yl)ethan-1-ol (85c)

To a solution of titanium tetrachloride (16.1 mL, 16.13 mmol, 1 M solution in DCM) in diethyl ether (70 mL) at −78° C. was added methyl lithium (1.6 M in THF, 10 mL, 16.129 mmol) and the temperature of the reaction mixture was slowly raised to −25° C. and stirred for 1 h. The reaction mixture was cooled to −50° C. and a solution of 2-chloro-5-nitroisonicotinaldehyde (1.0 g, 5.376 mmol) in diethyl ether (10 mL) was added to the reaction mixture and stirred at same temperature for 1 h. The progress of the reaction was monitored by TLC. The reaction mixture was quenched with saturated ammonium chloride solution and extracted with EtOAc (2×400 mL). The combined organic extracts were dried over anhydrous sodium sulfate, filtered and concentrated under reduced pressure to afford 1-(2-chloro-5-nitropyridin-4-yl)ethan-1-ol which was used in the following step without any further purification. Yield: 1.0 g (92%); ¹H-NMR (400 MHz, CDCl₃): δ 8.99 (s, 1H), 7.88 (s, 1H), 5.59 (q, J=6.4 Hz, 1H), 2.40 (bs, 1H), 1.56 (d, J=6.4 Hz, 3H)

### Step—d: Synthesis of 1-(2-chloro-5-nitropyridin-4-yl)ethan-1-one (85d)

To a solution of 1-(2-chloro-5-nitropyridin-4-yl)ethan-1-ol (1.0 g, 4.95 mmol) in DCM (40 mL) at ambient temperature was added Dess-Martin Periodinane (3.78 g, 8.91 mmol) and the mixture was stirred for 16 h. The progress of the reaction was monitored by TLC. The reaction mixture was diluted with cold water (50 mL) and extracted with DCM (2×100 mL). The combined organic extracts were dried over anhydrous sodium sulfate, filtered and concentrated under reduced pressure to afford 1-(2-chloro-5-nitropyridin-4-yl)ethan-1-one which was used in the following step without any further purification. Yield: 0.9 g (91%)¹H-NMR (400 MHz, CDCl3): δ 9.17 (s, 1H), 7.34 (s, 1H), 2.60 (s, 3H); LCMS: m/z 201.0 [M+H]⁺.

### Step—e: 1-(2-(4-ethylpiperazin-1-yl)-5-nitropyridin-4-yl)ethan-1-one (85e)

To a solution of 1-(2-chloro-5-nitropyridin-4-yl)ethan-1-one (900 mg, 4.5 mmol) in 1,4-Dioxane (10 mL) were added 1-ethylpiperazine (1.02 g, 9 mmol) and DIPEA (2.32 g, 18 mmol), then the microwave vial was capped and irradiated in microwave reactor at 100° C. for 1 h. The reaction mixture was diluted with water (30 mL) and extracted with EtOAc (2×100 mL). The combined organic extracts were dried over anhydrous sodium sulfate and evaporated under reduced pressure to afford 1-(2-(4-ethylpiperazin-1-yl)-5-nitropyridin-4-yl)ethan-1-one: 1.1 g (88%); LCMS: m/z 279.20 [M+H]⁺.

### Step—f: 1-(5-amino-2-(4-ethylpiperazin-1-yl)pyridin-4-yl)ethan-1-one (85f)

To a stirred solution of 1-(2-(4-ethylpiperazin-1-yl)-5-nitropyridin-4-yl)ethan-1-one (2.0 g, 7.194 mmol) in methanol (48 mL) and water (12 mL) at ambient temperature were added iron powder (2.01 g, 35.971 mmol) and ammonium chloride (3.84 g, 71.94 mmol). The reaction mixture was heated at 85° C. for 3 h. The progress of the reaction was monitored by TLC. The reaction mixture was filtered through celite bed and the filtrate was extracted with DCM (300 mL) and water (50 mL). The aqueous layer was extracted with DCM (2×200 mL), the combined organic layer was washed with water (30 mL), the aqueous layer was extracted with EtOAc (2×30 mL). The combined organic extracts were dried over Na₂SO₄ and concentrated under reduced pressure to afford 1-(5-amino-2-(4-ethylpiperazin-1-yl)pyridin-4-yl)ethan-1-one. Yield: 1.8 g (100%); LCMS: m/z 249.10 [M+H]⁺.

### Step—g: 6-(4-ethylpiperazin-1-yl)pyrido[3,4-c]pyridazin-4-ol (85g)

To a solution of afford 1-(5-amino-2-(4-ethylpiperazin-1-yl)pyridin-4-yl)ethan-1-one (1.8 g, 7.258 mmol) in Conc. Hydrochloric acid (18 mL) at 0° C. was added water (90 mL) stirred for 10 min. To the reaction mixture at 0° C. Sodium nitrite (0.6 g, 8.709 mmol) was added and stirred at same temperature for 1 h. The progress of the reaction was monitored by TLC. The reaction mixture was basified to pH˜ 7 with saturated sodium bicarbonate solution and extracted with ethyl acetate. Then the aqueous layer was concentrated to afford 4-chloro-6-(4-ethylpiperazin-1-yl)pyrido[3,4-c]pyridazine which was used in the following step without further purification. Yield: 620 mg (33%); LCMS: m/z 260.10 [M+H]⁺.

### Step—h: 4-chloro-6-(4-ethylpiperazin-1-yl)pyrido[3,4-c]pyridazine (85h)

To a solution of 6-(4-ethylpiperazin-1-yl)pyrido[3,4-c]pyridazin-4-ol (600 mg, 2.316 mmol) in acetonitrile (30 mL) were added phosphorus(V) oxychloride (1.63 mL, 6.949 mmol) and diisopropylethylamine (0.80 mL, 4.633 mmol) at RT and the reaction mixture was stirred at 100° C. for 3 h. The progress of the reaction was monitored by TLC. The reaction mixture was cooled to 0° C. and basified with saturated sodium bicarbonate solution then extracted with EtOAc (2×500 mL). The combined organic extracts were dried over anhydrous sodium sulfate and concentrated under reduced pressure. The residue obtained was purified by flash column chromatography using 3% methanol in DCM as an eluent to afford 4-chloro-6-(4-ethylpiperazin-1-yl)pyrido[3,4-c]pyridazine as a yellow solid. Yield: 270 mg (42%); LCMS: m/z 278.0 [M+H]⁺.

### Step—i: (R)-6-(4-ethylpiperazin-1-yl)-N-(1-(3-nitro-5-(trifluoromethyl)phenyl) ethyl)pyrido[3,4-c]pyridazin-4-amine (85i)

To a solution of 4-chloro-6-(4-ethylpiperazin-1-yl)pyrido[3,4-c]pyridazine (270 mg, 0.974 mmol) in toluene (5 mL) were added (R)-1-(3-nitro-5-(trifluoromethyl)phenyl)ethan-1-amine hydrochloride (342 mg, 1.462 mmol), sodium tert-butoxide (187 mg, 1.949 mmol), then the reaction mixture was purged with nitrogen gas for 5 min. To the reaction mixture xantphos (68 mg, 0.146 mmol) and Tris(dibenzylideneacetone)dipalladium(0) was added and purged with nitrogen gas for 2 min, then the microwave vial was capped and irradiated in microwave reactor at 120° C. for 1 h. The reaction mixture was diluted with water (30 mL) and extracted with EtOAc (2×100 mL). The combined organic extracts were dried over anhydrous sodium sulfate and evaporated under reduced pressure. The residue was purified by flash column chromatography using 5% methanol in DCM as an eluent to afford (R)-6-(4-ethylpiperazin-1-yl)-N-(1-(3-nitro-5-(trifluoromethyl)phenyl) ethyl)pyrido[3,4-c]pyridazin-4-amine. Yield: 130 mg (impure); LCMS: m/z 476.20 [M+H]⁺.

### Step—j: (R)—N-(1-(3-amino-5-(trifluoromethyl)phenyl)ethyl)-6-(4-ethylpiperazin-1-yl)pyrido[3,4-c]pyridazin-4-amine

The title compound was synthesized using the same procedure which was followed for (R)—N-(1-(3-amino-5-(trifluoromethyl)phenyl)ethyl)-2-chloro-6-(4-ethylpiperazin-1-yl)pyrido[2,3-d]pyrimidin-4-amine (Example—80) by using (R)-6-(4-ethylpiperazin-1-yl)-N-(1-(3-nitro-5-(trifluoromethyl)phenyl)ethyl)pyrido[3,4-c]pyridazin-4-amine as starting material (Yield: 8%); ¹H-NMR (400 MHz, DMSO-d₆): δ 9.19 (s, 1H), 8.14 (s, 1H), 8.42 (d, J=8.0 Hz, 1H), 7.32 (s, 1H), 6.88 (s, 1H), 6.80 (s, 1H), 6.71 (s, 1H), 5.50 (bs, 2H), 4.86-4.83 (m, 1H), 3.69 (bs, 4H), 2.50 (bs, 4H), 2.42 (q, J=7.2 Hz, 2H), 1.59 (d, J=6.8 Hz, 3H), 1.09 (t, J=8.0 Hz, 3H); LCMS: m/z 446.20 [M+H]⁺.

### Example—86: (R)—N-(1-(3-amino-5-(trifluoromethyl)phenyl)ethyl)-3-(4-ethylpiperazin-1-yl)-8-methylpyrido[2,3-d]pyridazin-5-amine

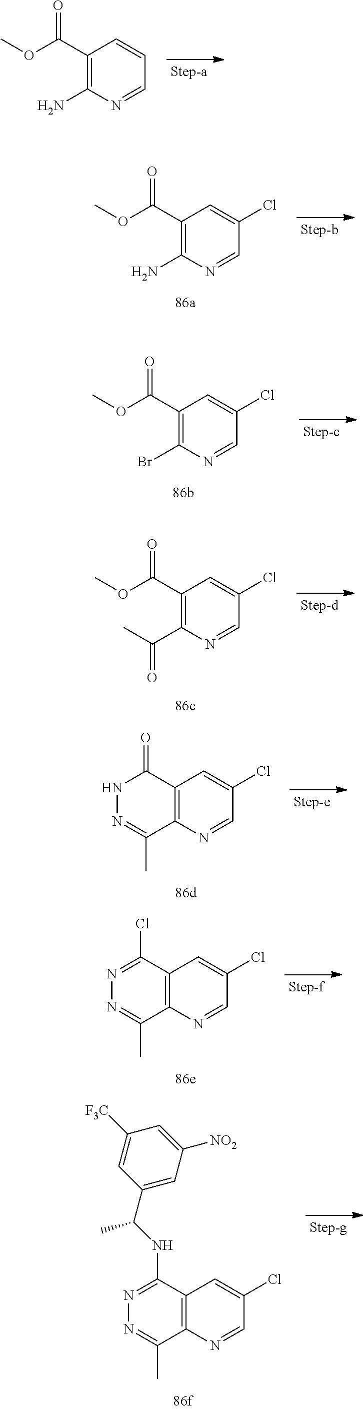

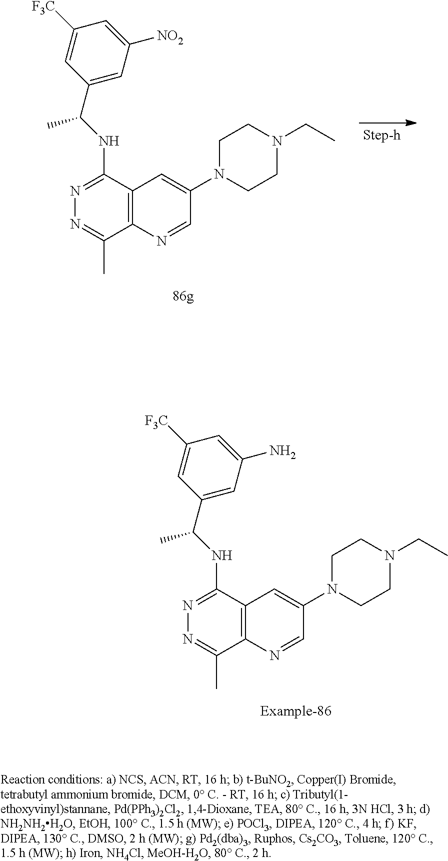

### Step—a: methyl 2-amino-5-chloronicotinate (86a)

To a solution of methyl 2-aminonicotinate (10 g, 65.724 mmol) in Acetonitrile (120 mL) was added N-Chlorosuccinimide (10.48 g, 78.869 mmol) by portions and the contents of the reaction mixture was stirred at 60° C. for 16 h. The progress of the reaction was monitored by TLC. The reaction mixture was diluted with saturated sodium bicarbonate solution and extracted with EtOAc (3×200 mL). The combined organic extracts were washed with water (50 mL), brine (50 mL), dried over anhydrous Na₂SO₄ and concentrated under reduced pressure to afford methyl 2-amino-5-chloronicotinate which was used in the following step without any further purification. Yield: 12.16 g (100%). ¹H-NMR (400 MHz, CDCl₃): δ 8.17 (d, J=2.4 Hz, 1H), 8.10 (d, J=2.4 Hz, 1H), 6.47 (bs, 2H), 3.90 (s, 3H); LCMS: m/z 186.9 [M+H]⁺.

### Step—b: methyl 2-bromo-5-chloronicotinate (86b)

To a solution of methyl 2-amino-5-chloronicotinate (5.3 g, 28.494 mmol) in Acetonitrile (100 mL) at 0° C. were added tert-butyl nitrite (5.8 g, 56.989 mmol), Copper(I) bromide (4.9 g, 34.193 mmol) and tetrabutylammonium bromide (918 mg, 2.849 mmol). The contents of the reaction mixture were stirred at RT for 16 h. The progress of the reaction was monitored by TLC. The reaction mixture was diluted with water (150 mL) and extracted with EtOAc (3×200 mL). The combined organic layer was washed with brine (100 mL), dried over anhydrous Na₂SO₄ and concentrated under reduced pressure. The residue was purified by flash column chromatography using 25% of EtOAc in hexane as an eluent to afford methyl 2-bromo-5-chloronicotinate. Yield: 1.35 g (19%). LCMS: m/z 251.8 [M+H]⁺.

### Step—c: Synthesis of methyl 2-acetyl-5-chloronicotinate (86c)

To a solution of methyl 2-bromo-5-chloronicotinate (1.35 g, 5.39 mmol) in 1,4-Dioxane (15 mL) were added Tributyl(1-ethoxyvinyl)stannane (2.14 g, 5.93 mmol) and triethyl amine (1.91 mL, 13.47 mmol). The reaction mixture was purged with nitrogen gas for 2 min and followed by the addition of Bis(triphenylphosphine)palladium(II) dichloride (75 mg, 0.108 mmol) the seal tube was capped and heated at 80° C. for 16 h. The reaction mixture was cooled to RT, acidified with 5 N HCl (15 mL) and stirred for 10 min. The reaction mixture was diluted with water (30 mL), extracted with EtOAc (2×100 mL). The combined organic extracts were washed with brine (30 mL), dried over anhydrous Na₂SO₄ and concentrated under reduced pressure. The residue was purified by combi flash column chromatography using 20% of EtOAc in hexane as an eluent to afford methyl 2-acetyl-5-chloronicotinate. Yield: 850 mg (70%). ¹H-NMR (400 MHz, CDCl₃): δ 8.65 (d, J=2.0 Hz, 1H), 7.94 (d, J=2.4 Hz, 1H), 3.93 (s, 3H), 2.67 (s, 3H); LCMS: m/z 214.0 [M+H]⁺.

### Step—d: 3-chloro-8-methylpyrido[2,3-d]pyridazin-5(6H)-one (86d)

To a solution of methyl 2-acetyl-5-chloronicotinate (850 mg, 3.98 mmol) in ethanol (15 mL) was added hydrazine monohydrate (1.5 mL) and the microwave vial was capped and irradiated in microwave reactor at 100° C. for 1.5 h. The reaction mixture was diluted with water (40 mL) and extracted with EtOAc (3×50 mL). The combined organic layers were washed with brine (30 mL), dried over anhydrous sodium sulfate and evaporated under reduced pressure. The residue was purified by flash column chromatography using 25% EtOAc in hexane as an eluent to afford 3-chloro-8-methylpyrido[2,3-d]pyridazin-5(6H)-one. Yield: 400 mg (27%); LCMS: m/z 196.0 [M+H]⁺.

### Step—e: 3,5-dichloro-8-methylpyrido[2,3-d]pyridazine (86e)

To a solution of 3-chloro-8-methylpyrido[2,3-d]pyridazin-5(6H)-one (400 mg, 1.011 mmol) in phosphorus(V) oxychloride (8 mL) was added diisopropylethylamine (0.5 mL, 3.03 mmol) at RT and the reaction mixture was stirred at 120° C. for 3 h. The progress of the reaction was monitored by TLC. Phosphorus(V)oxychloride was distilled off under reduced pressure. The residue obtained was basified with saturated sodium bicarbonate solution and extracted with EtOAC (3×40 mL). The combined organic extracts were washed with brine solution (50 mL), dried over anhydrous sodium sulfate and concentrated under reduced pressure. The residue was purified by flash column chromatography using 30% EtOAC in hexane as an eluent to afford 3,5-dichloro-8-methylpyrido[2,3-d]pyridazine. Yield: 260 mg (60%); LCMS: m/z 214.0 [M+H]⁺.

### Step—f: Synthesis of (R)-3-chloro-8-methyl-N-(1-(3-nitro-5-(trifluoromethyl) phenyl)ethyl)pyrido[2,3-d]pyridazin-5-amine (86f)

To a solution of 3,5-dichloro-8-methylpyrido[2,3-d]pyridazine (300 mg, 1.41 mmol) in DMSO (5 mL) were added (R)-1-(3-nitro-5-(trifluoromethyl)phenyl)ethan-1-amine hydrochloride (494 mg, 2.11 mmol), Potassium fluoride (245 mg, 4.22 mmol) and DIPEA (0.5 mL, 2.82 mmol) then the microwave vial was capped and irradiated in microwave reactor at 130° C. for 2 h. The reaction mixture was diluted with water (30 mL) and extracted with EtOAc (2×50 mL). The combined organic extracts were washed with brine (30 mL), dried over anhydrous sodium sulfate and evaporated under reduced pressure. The residue was purified by flash column chromatography using 25% EtOAc in hexane as an eluent to afford (R)-3-chloro-8-methyl-N-(1-(3-nitro-5-(trifluoromethyl)phenyl) ethyl)pyrido[2,3-d]pyridazin-5-amine. Yield: 250 mg (43%); LCMS: m/z 412.1 [M+H]⁺.

### Step—g: Synthesis of (R)-3-(4-ethylpiperazin-1-yl)-8-methyl-N-(1-(3-nitro-5-(trifluoromethyl)phenyl)ethyl)pyrido[2,3-d]pyridazin-5-amine (86g)

To a solution of (R)-3-chloro-8-methyl-N-(1-(3-nitro-5-(trifluoromethyl)phenyl) ethyl)pyrido[2,3-d]pyridazin-5-amine (70 mg, 0.17 mmol) in Toluene (2 mL) were added 1-ethylpiperazine (39 mg, 0.34 mmol) and cesium carbonate (166 mg, 0.51 mmol), the reaction mixture was purged with nitrogen gas for 3 min. To the mixture RuPhos (15.8 mg, 0.034 mmol) and Tris(dibenzylideneacetone)dipalladium(0) (15.6 mg, 0.017 mmol) was added and the microwave vial was capped and irradiated in microwave reactor at 120° C. for 1.5 h. The reaction mixture was diluted with water (20 mL) and extracted with EtOAc (3×20 mL). The combined organic extracts were washed with brine (20 mL), dried over anhydrous sodium sulfate and evaporated under reduced pressure. The residue was purified by flash column chromatography using 10% MeOH in DCM as an eluent to afford (R)-3-(4-ethylpiperazin-1-yl)-8-methyl-N-(1-(3-nitro-5-(trifluoromethyl)phenyl)ethyl)pyrido[2,3-d]pyridazin-5-amine. Yield: 35 mg (42%); LCMS: m/z 490.2 [M+H]⁺.

### Step—h: Synthesis of (R)—N-(1-(3-amino-5-(trifluoromethyl)phenyl)ethyl)-3-(4-ethylpiperazin-1-yl)-8-methylpyrido[2,3-d]pyridazin-5-amine

The title compound was synthesized using the same procedure which was followed for (R)—N-(1-(3-amino-5-(trifluoromethyl)phenyl)ethyl)-2-chloro-6-(4-ethylpiperazin-1-yl)pyrido[2,3-d]pyrimidin-4-amine (Example—80) by using (R)-3-(4-ethylpiperazin-1-yl)-8-methyl-N-(1-(3-nitro-5-(trifluoromethyl)phenyl)ethyl)pyrido[2,3-d]pyridazin-5-amine as starting material (Yield: 36%); ¹H-NMR (400 MHz, CDCl₃): δ 9.06 (d, J=2.4 Hz, 1H), 8.01 (s, 1H), 7.65 (bs, 1H), 6.84 (d, J=5.6 Hz, 2H), 6.67 (s, 1H), 5.51 (bs, 2H), 5.36-5.32 (m, 1H), 3.61 (bs, 4H), 3.33 (2H, merged with DMSO moisture peak), 2.80 (bs, 4H), 2.62 (s, 3H), 1.57 (d, J=6.8 Hz, 3H), 1.14 (t, J=6.8 Hz, 3H); LCMS: m/z 460.2 [M+H]⁺.

TABLE 10
Compound in table-10 was prepared by using process analogous to the
preparation of example-86

| | | | 1H NMR (400 MHz, |
|---|---|---|---|
| | | | DMSO-d₆): δppm |
| Example # | Structure | IUPAC Name | LC-MS: m/z |
| 87 | 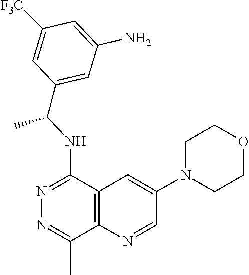 [SMILES: Cc1nnc(N[C@H](C)c2cc(N)cc(C(F)(F)F)c2)c2cc(N3CCOCC3)cnc12] | (R)-N-(1-(3-amino-5- (trifluoromethyl)phenyl) ethyl)-8-methyl-3- morpholinopyrido[2,3- d]pyridazin-5-amine | 9.02 (d, J = 2.4 Hz, 1H), 7.95 (d, J = 2.4 Hz, 1H), 7.42 (d, J = 7.2 Hz, 1H), 6.83 (s, 2H), 6.66 (s, 1H), 5.50 (s, 2H), 5.37 (quin, J = 7.2 Hz, 1H), 3.84 (t, J = 4.8 Hz, 4H), 3.46 (t, J = 4.8 Hz, 4H), 2.60 (s, 3H), 1.54 (d, J = 7.2 Hz, 3H) 433.30 [M + H]⁺ |

### Example—88: (R)—N-(1-(3-amino-5-(trifluoromethyl)phenyl)ethyl)-7-chloro-3-(4-ethylpiperazin-1-yl)-1,6-naphthyridin-5-amine

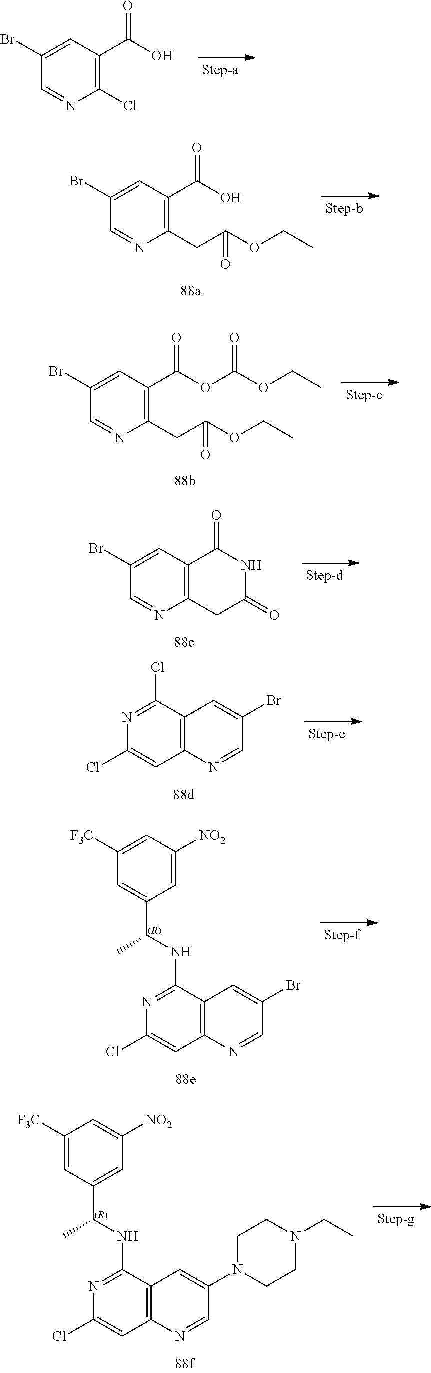

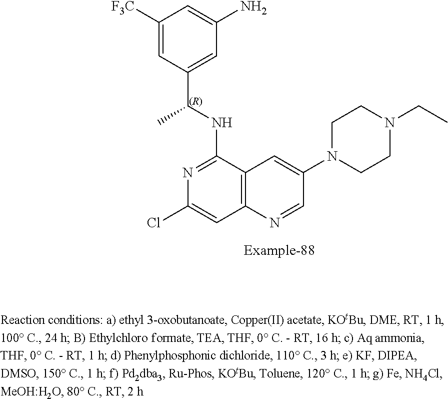

### Step—a: 5-bromo-2-(2-ethoxy-2-oxoethyl)nicotinic acid (88a)

To a solution of ethyl 3-oxobutanoate (8.1 mL, 63.44 mmol) in DME (300 mL) at RT was added potassium tert-butoxide (7.12 g, 63.44 mmol) and stirred for 1 h. To the reaction mixture 5-bromo-2-chloronicotinic acid (10 g, 42.29 mmol) and copper(II) acetate (11.52 g, 63.44 mmol) was added and stirred for 24 h at 100° C. The progress of the reaction was monitored by TLC. The reaction mixture was diluted with water (100 mL) and extracted with EtOAc (3×100 mL). The combined organic layers were washed with brine (150 mL), dried over anhydrous sodium sulfate and evaporated under reduced pressure. The residue was purified by flash column chromatography using 70% EtOAc in Hexane as an eluent to afford bromo-2-(2-ethoxy-2-oxoethyl)nicotinic acid. Yield: 5.8 g; Crude compound was used in the next step without any confirmation.

### Step—b: 5-bromo-2-(2-ethoxy-2-oxoethyl)nicotinic (ethyl carbonic) anhydride (88b)

To a solution of 5-bromo-2-(2-ethoxy-2-oxoethyl)nicotinic acid (1.8 g, 6.30 mmol) in THF (30 mL) was added TEA (3.5 ml, 25.2 mmol) at 0° C. and stirred for 10 min, then Ethyl chloroformate (1.2 ml, 12.5 mmol) was added and stirred for 16 h at RT. The reaction mixture was diluted with water (20 mL) and extracted with EtOAc (3×30 mL). The combined organic layers were washed with brine (50 mL), dried over anhydrous sodium sulfate and evaporated under reduced pressure. The residue was purified by flash column chromatography using 20% EtOAc in hexane as an eluent to afford 5-bromo-2-(2-ethoxy-2-oxoethyl)nicotinic (ethyl carbonic) anhydride as a yellow color solid. Yield: 560 mg; Compound was used in the next step without any confirmation.

### Step—c: 3-bromo-1,6-naphthyridine-5,7(6H,8H)-dione (88c)

To a solution of 5-bromo-2-(2-ethoxy-2-oxoethyl)nicotinic (ethyl carbonic) anhydride (0.71 g, 1.97 mmol) in THF (10 mL) was added aq. Ammonia solution (2.1 mL) at 0° C. and stirred for 1h. The precipitated solid was filtered off and dried under vacuum to afford 3-bromo-1,6-naphthyridine-5,7(6H,8H)-dione as a yellow solid. Yield: 440 mg (97%); LCMS: m/z 241.20 [M+H]⁺.

### Step—d: 3-bromo-5,7-dichloro-1,6-naphthyridine (88d)

A mixture of 3-bromo-1,6-naphthyridine-5,7(6H,8H)-dione (1.1 g, 4.5 mmol) and phenylphosphonic dichloride (11 ml) was stirred at 110° for 3 h. Then reaction mixture was quenched with water (20 mL) and extracted with EtOAc (3×30 mL). The combined organic layers were washed with Sat NaHCO₃ (30 mL) and brine (50 mL), dried over anhydrous sodium sulfate and evaporated under reduced pressure. The residue was purified by flash column chromatography using 2% MeOH in DCM as an eluent to afford the title compound as a yellow solid. Yield: 690 mg (55%); LCMS: m/z 276.80 [M+H]⁺.

### Step—e: (R)-3-bromo-7-chloro-N-(1-(3-nitro-5-(trifluoromethyl)phenyl)ethyl)-1,6-naphthyridin-5-amine (88e)

To a solution of 3-bromo-5,7-dichloro-1,6-naphthyridine (780 mg, 2.8 mmol) in DMSO (10 mL) were added (R)-1-(3-nitro-5-(trifluoromethyl)phenyl)ethan-1-amine hydrochloride (916 mg, 3.39 mmol), KF (162 mg, 28 mmol) and DIPEA (1.45 mL, 8.4 mmol) and Microwave vial was capped irradiated at 150° C. for 1 h in microwave reactor. The reaction mixture was diluted with water (20 mL) and extracted with EtOAc (3×20 mL). The combined organic extracts were washed with brine (20 mL), dried over anhydrous sodium sulfate and evaporated under reduced pressure. The residue was purified by flash column chromatography using 10% EtOAc in hexane as an eluent to afford ((R)-3-bromo-7-chloro-N-(1-(3-nitro-5-(trifluoromethyl)phenyl)ethyl)-1,6-naphthyridin-5-amine as a yellow solid. Yield: 1.0 g (77%); ¹H-NMR (400 MHz, DMSO-d₆): δ 9.16 (d, J=1.2 Hz, 1H), 8.99 (d, J=2.0 Hz, 1H), 8.61 (s, 1H), 8.58 (d, J=6.8 Hz, 1H), 8.35 (s, 1H), 8.34 (s, 1H), 7.01 (s, 1H), 5.52-5.47 (m, 1H), 1.66 (d, J=6.8 Hz, 3H); LCMS: m/z 475.40 [M+H]⁺.

### Step—f: (R)-7-chloro-3-(4-ethylpiperazin-1-yl)-N-(1-(3-nitro-5-(trifluoromethyl) phenyl)ethyl)-1,6-naphthyridin-5-amine (88f)

To a solution of ((R)-3-bromo-7-chloro-N-(1-(3-nitro-5-trifluoromethyl)phenyl)ethyl)-1,6-naphthyridin-5-amine (50 mg, 0.105 mmol) in Toluene (2 mL) were added 1-ethylpiperazine (0.027 mL, 0.211 mmol), RuPhos (5 mg, 0.0105 mmol), Tris(dibenzylideneacetone)dipalladium(0) (8 mg, 0.0084 mmol) and potassium tert-butoxide (24 mg, 0.211 mmol), then the reaction mixture was purged with nitrogen gas for 5 min. The microwave vial was capped and irradiated at 120° C. for 1 h in microwave reactor. The reaction mixture was diluted with water (20 mL) and extracted with EtOAc (3×20 mL). The combined organic extracts were washed with brine (20 mL), dried over anhydrous sodium sulfate and evaporated under reduced pressure. The residue was purified by flash column chromatography using 5% methanol in DCM as an eluent to afford (R)-7-chloro-3-(4-ethylpiperazin-1-yl)-N-(1-(3-nitro-5-(trifluoromethyl)phenyl)ethyl)-1,6-naphthyridin-5-amine as a brown solid. Yield:15 mg (30%); ¹H-NMR (400 MHz, DMSO-d₆): δ8.90 (s, 1H), 8.58 (s, 1H), 8.35 (s, 1H), 8.32 (s, 1H) 8.24 (d, J=6.8 Hz, 1H), 7.96 (s, 1H), 6.91 (s, 1H), 5.53-5.50 (m, 1H), 2.60 (bs, 4H), 2.40 (bs, 4H), 2.40 (2H merged with DMSO solvent peak), 1.67 (d, J=6.8 Hz, 3H), 1.09 (t, J=7.2 Hz, 3H); LCMS: m/z 509.10 [M+H]⁺.

### Step—g: (R)—N-(1-(3-amino-5-(trifluoromethyl)phenyl)ethyl)-7-chloro-3-(4-ethylpiperazin-1-yl)-1,6-naphthyridin-5-amine

The title compound was synthesized using the same procedure which was followed for (R)—N-(1-(3-amino-5-(trifluoromethyl)phenyl)ethyl)-2-chloro-6-(4-ethylpiperazin-1-yl)pyrido[2,3-d]pyrimidin-4-amine (Example—80) by using (R)-7-chloro-3-(4-ethylpiperazin-1-yl)-N-(1-(3-nitro-5-(trifluoromethyl)phenyl)ethyl)-1,6-naphthyridin-5-amine as starting material. Yield: 12 mg (13%); ¹H-NMR (400 MHz, DMSO-d₆): δ 8.87 (s, 1H), 8.05 (d, J 8.0 Hz, 1H), 8.0 (s, 1H), 6.87 (s, 1H), 6.86 (s, 1H), 6.83 (s, 1H), 6.69 (s, 1H), 5.50 (bs, 2H), 5.38-5.34 (i, 1H), 2.57 (bs, 6H), 2.40 (bs, 4H), 1.55 (d, J 6.8 Hz, 3H), 1.07 (t, J(=7.2 Hz, 3H); LCMS: m/z 479.20 [M+H]J.

TABLE 11
Compound in table-11 was prepared by using process analogous to the
preparation of example-88

| | | | 1H NMR (400 MHz, |
|---|---|---|---|
| | | | DMSO-d₆): δppm |
| Example # | Structure | IUPAC Name | LC-MS: m/z |
| 89 | 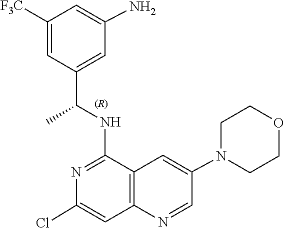 | (R)-N-(1-(3-amino-5- (trifluoromethyl) phenyl)ethyl)-7- chloro-3- morpholino-1,6- naphthyridin-5-amine | 8.89 (d, J = 2.40 Hz, 1H), 8.07 (d, J = 7.6 Hz, 1H), 8.0 (s, 1H), 6.88 (s, 1H), 6.86 (s, 1H), 6.83 (s, 1H), 6.69 (s, 1H), 5.50 (bs, 2H), 5.38-5.34 (m, 1H), 3.38 (t, J = 4.4 Hz, 4H), 3.48 (4H merged with DMSO solvent peak), 1.55 (d, J = 6.8 Hz, 3H); LCMS: m/z 452.20 [M + H]⁺. |

### List of Abbreviations Used in the Present Invention are Given Below

ACN Acetonitrile
AcOH Acetic acid
Aq Aqueous
bs broad singlet
BSA Bovine Serum Albumin
° C. degree Celsius
CS₂CO₃ Cesium carbonate
d doublet
DCM Dichloromethane
dd doublet of doublets
DIPEAN, N-Diisopropylethylamine
DME Dimethoxy ethane
DMF Dimethylformamide
DMF-DMA N,N-Dimethylformamide dimethyl acetal
DMSODimethyl sulfoxide
DTT Dithiothreitol
EDA-GTP-DY-647P1 2′/3′—O-(2-Aminoethyl-carbamoyl)-Guanosine-5′-triphosphate, labeled with DY-647P1,Triethylammoniumsalt
EtOAc Ethyl acetate
EtOH Ethanol
FRET Fluorescence resonance energy transfer
g gram
GDP Guanosine diphosphate
GEF Guanine nucleotide exchange factor
GTP Guanosine triphosphate
h hour
HATU (1-[Bis(dimethylamino)methylene]-1H-1,2,3-triazolo[4,5-b]pyridinium 3-oxide hexafluorophosphate
HEPES (4-(2-hydroxyethyl)-1-piperazineethanesulfonic acid)
HPLC High-performance liquid chromatography
HTRF Homogeneous Time Resolved Fluorescence
IPA Isopropanol
K₂CO₃ Potassium carbonate
KRAS Kirsten Rat Sarcoma
KO′Bu Potassium tert-butoxide
LC Liquid chromatography
LiOH·H₂O Lithium hydroxide monohydrate
m multiplet
MeOH Methanol
MeONa Sodium methoxide
mg milligram
MHz Megahertz
min minutes
mL milliliter
mM millimolar
mmol Millimoles
MS Mass spectrometry
NaBH₄ Sodium borohydride
NaNO₂ Sodium nitrite
NaIO4 Sodium periodate
NH₄Cl Ammonium chloride
NiCl₂·6H₂O Nickel(II) chloride hexahydrate
nM nanomolar
NMR Nuclear magnetic resonance spectroscopy
O/N Over night
Pd—C Palladium on carbon
Pd(PPh₃)₄ Tetrakis(triphenylphosphine)palladium(O)
POCl₃ Phosphorus oxychloride
prep Preparative
RT Room temperature
RuPhos 2-Dicyclohexylphosphino-2′,6′-diisopropoxybiphenyl
SOS1 Son of sevenless 1
t triplet
TEA Triethylamine
THF Tetrahydrofuran
TFA Trifluoroacetic acid
TiCl₄ Titanium tetrachloride
TLC Thin-layer chromatography

### Pharmacological Activity:

### Biochemical Assay: Measurement of SOS1-Mediated KRAS Guanine Nucleotide Exchange (GEF) Assay

This assay quantifies the HTRF-based SOS1 mediated guanine nucleotide exchange assay detecting GTP binding to KRAS (G12C), GST-Tag, GDP-bound protein. This is also referred as “On Assay”.The assay buffer composition is as follows: 10 mM HEPES pH-7, 150 mM NaCl, 5 mM MgCl2, 1 mM DTT, 0.05% BSA, 0.0025% IGEPAL CA-630 and MilliQ water. 0.5 μl of compounds diluted in assay buffer were dispensed into the respective wells of 384 black well test plate. 4.75 μl of KRAS working solution containing 100 nM KRAS (G12C), GST-Tag, GDP-bound protein GDP-loaded (Reaction biology) and 2 nM Anti GST-Tb cryptate (FRET donor, Cisbio) was added in assay buffer. The components were incubated for 10 min at 25° C.

4.75 μl of hSOS1 working solution containing 20 nM hSOS1 enzyme and 200 nM EDA-GTP-DY-647P (FRET acceptor, synthesized by Jena Bioscience (Germany), supplied as a 1 mM solution) was further added in assay buffer. 4.75 μl of inhibitor control having only 200 nM EDA-GTP-DY-647P1 component without hSOS1 working solution was added in the negative control well. The plate was short spun for few seconds and incubated for 10 min. HTRF was measured using Perkin Elmer-Nivo 5F multimode plate reader.

HTRF settings: EXCITATION FILTER—320/75 nm, EMISSION FILTER (1) —615/8 nm, EMISSION FILTER (2)—665/8 nm

The compounds when tested for their potential to inhibit guanine nucleotide exchange (GEF) in the above-mentioned assay exhibited inhibition. The IC50 (μM) values of inhibition of this guanine nucleotide exchange (GEF) for some of the exemplary compounds are given in the below table:

| | GEF assay |
|---|---|
| Example # | % inhibition @ 1 μM |
| 1 | + |
| 2 | + |
| 3 | + |
| 4 | + |
| 5 | ++ |
| 6 | ++ |
| 7 | + |
| 8 | + |
| 9 | + |
| 10 | + |
| 11 | + |
| 12 | + |
| 13 | + |
| 14 | + |
| 15 | + |
| 16 | + |
| 17 | + |
| 18 | + |
| 19 | + |
| 20 | + |
| 21 | + |
| 22 | + |
| 23 | + |
| 24 | + |
| 25 | + |
| 26 | + |
| 27 | + |
| 28 | + |
| 29 | + |
| 30 | + |
| 31 | + |
| 32 | + |
| 33 | + |
| 34 | + |
| 35 | + |
| 36 | + |
| 37 | + |
| 38 | + |
| 39 | + |
| 40 | + |
| 41 | + |
| 42 | + |
| 43 | + |
| 44 | + |
| 45 | + |
| 46 | + |
| 47 | ++ |
| 48 | + |
| 49 | + |
| 50 | + |
| 51 | + |
| 52 | + |
| 53 | + |
| 54 | + |
| 55 | + |
| 56 | + |
| 57 | + |
| 58 | + |
| 59 | + |
| 60 | + |
| 61 | + |
| 62 | + |
| 63 | + |
| 64 | + |
| 65 | + |
| 66 | + |
| 67 | + |
| 68 | + |
| 69 | + |
| 70 | + |
| 71 | + |
| 72 | + |
| 73 | + |
| 74 | + |
| 75 | ++ |
| 76 | ++ |
| 77 | + |
| 78 | + |
| 79 | + |
| 80 | + |
| 81 | + |
| 82 | + |
| 83 | + |
| 84 | + |
| 85 | + |
| 86 | + |
| 87 | + |
| 88 | + |
| 89 | + |

Table key: ≥50% +; & <50% ++

1. Novel compounds of formula (I): 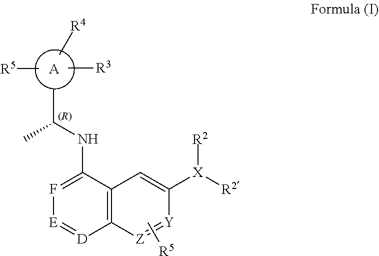
or a pharmaceutically acceptable salt or a pharmaceutically acceptable regioisomer thereof; wherein,
A is selected from 6-10 membered aryl, 5-10 membered heteroaryl and 3-10 membered heterocyclyl;
D, E, F is either CR¹ or N, with the option that either one or two of D, E, F are N and the rest being CR¹;
R¹ is selected from the group consisting of hydrogen, halogen, alkyl, alkenyl, alkynyl, —C(O)alkyl, cycloalkyl, heterocyclyl, aryl, heteroaryl, OR^{1a}, NR^{1a}R^{1b} wherein any of the group is optionally substituted by one or more, identical or different substituents, with the exclusion that R¹ is not alkyl when E is CR¹;
R^{1a} is selected from the group consisting of hydrogen, alkyl, —C(O)alkyl, —S(O)˜alkyl, —S(O)˜NH₂, alkenyl, alkynyl, cycloalkyl, heterocyclyl, aryl and heteroaryl, wherein any of the group is optionally substituted by one or more, identical or different substituents;
R^{1b} is selected from the group consisting of hydrogen, alkyl, —C(O)alkyl, cycloalkyl, heterocyclyl, aryl and heteroaryl, wherein any of the group is optionally substituted by one or more, identical or different substituents;
or R^{1a} and R^{1b} when present on a nitrogen taken together with the nitrogen atom to which they are attached form an optionally substituted 4-6 membered heterocyclyl containing 0-2 additional heteroatoms independently selected from O and N which is optionally substituted by one or more, identical or different substituents;
R^{1c} is selected from the group consisting of hydrogen, alkyl, —C(O)alkyl, alkenyl, alkynyl, cycloalkyl, heterocyclyl, aryl and heteroaryl, wherein any of the group is optionally substituted by one or more, identical or different substituents;
R^{1d} is selected from the group consisting of hydrogen, halogen, nitro, cyano, hydroxy, hydroxyl alkyl, amino, alkyl, alkoxy, alkenyl, alkynyl, wherein any of the group is optionally substituted by one or more, identical or different substituents;
R^{1e} is selected from the group consisting of hydrogen, halogen, nitro, cyano, hydroxy, hydroxyl alkyl, amino, alkyl, alkoxy, alkenyl, alkynyl, keto wherein any of the group is optionally substituted by one or more, identical or different substituents;
X is N or C(R^{1a}) or OR^{1c}, wherein R^{1a} and R^{1c} are as defined above;
Y is N or C(R^{1d}), wherein R^{1d} is as defined above;
Z is N or —C(O)— or C(R^{1c}), wherein R^{1c} is as defined above;
R² and R^{2′} are may be present or absent and when present are same or different and each individually is selected from the group consisting of hydrogen, halogen, alkyl, alkenyl, alkynyl, —C(O)alkyl, —C(═O), cycloalkyl, heterocyclyl, aryl, heteroaryl, OR^{1a} NR^{1a}R^{1b} wherein any of the group is optionally substituted by one or more, identical or different substituents; or R² and R^{2′} taken together with the atom to which they are attached form an optionally substituted 3-6 membered cycloalkyl, an optionally substituted 4-6 membered heterocyclyl containing 0-2 additional heteroatoms independently selected from O and N which is optionally substituted by one or more identical or different substituents;
R³ is selected from the group consisting of hydrogen, halogen, amino, nitro, alkyl, alkenyl, alkynyl, cycloalkyl, heterocyclyl, heteroaryl, wherein any of the group is optionally substituted by one or more, identical or different substituents;
R⁴ and R⁵ are same or different and each individually is selected from the group consisting of hydrogen, halogen, amino, nitro, alkyl or taken together when present on adjacent carbon atoms form an optionally substituted 4-6 membered aryl ring system containing optionally 1-2 heteroatoms independently selected from O and N.

2. The compound of formula (I) according to claim 1, or a pharmaceutically acceptable salt or a stereoisomer thereof, in which the group
is specifically represented by

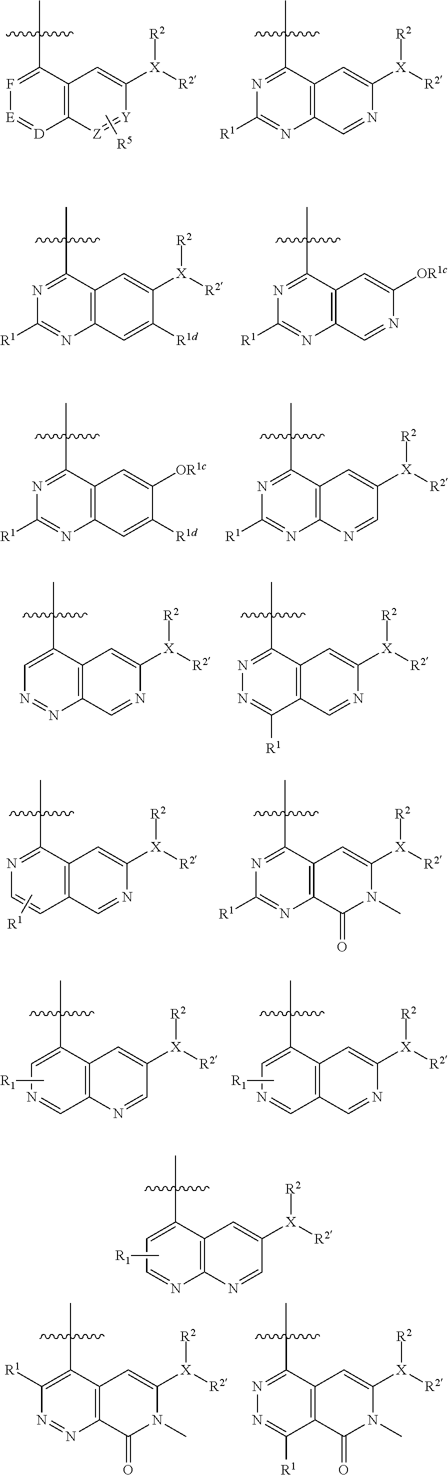

3. The compound of formula (I) according to claim 1, or a pharmaceutically acceptable salt or a stereoisomer thereof, in which the group
is specifically represented by

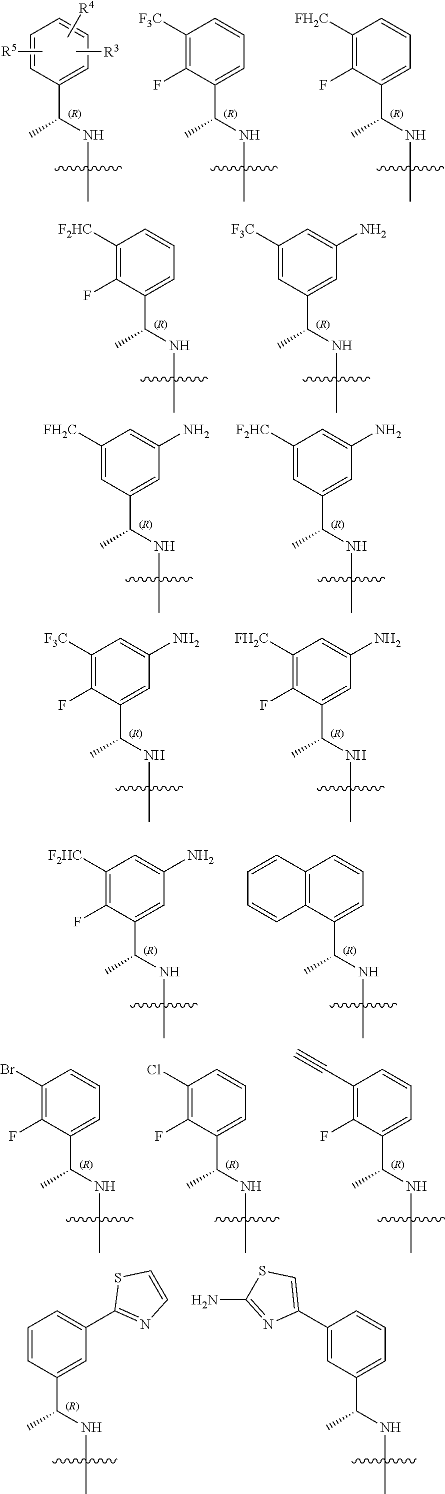

4. The compound of formula (I) according to claim 1, are to novel compounds of formula (Ia) 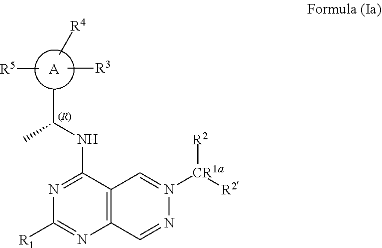
or a pharmaceutically acceptable salt or a pharmaceutically acceptable regioisomer thereof;
wherein A, the groups R¹, R², R^{2′}, R³, R⁴, R⁵ are as defined in claim 1.

5. The compound of formula (I) according to claim 1, are to novel compounds of formula (Ib) 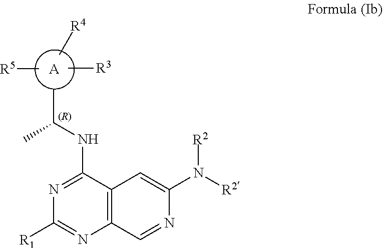
or a pharmaceutically acceptable salt or a pharmaceutically acceptable regioisomer thereof;
wherein A, the groups R¹, R², R^{2′}, R³, R⁴, R⁵ are as defined in claim 1.

6. The compound of formula (I) according to claim 1, are to novel compounds of formula (Ic) 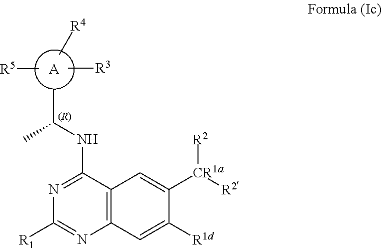
or a pharmaceutically acceptable salt or a pharmaceutically acceptable regioisomer thereof;
wherein A, the groups R¹, R², R^{2′}, R³, R⁴, R⁵ are as defined in claim 1.

7. The compound of formula (I) according to claim 1, are to novel compounds of formula (Id) 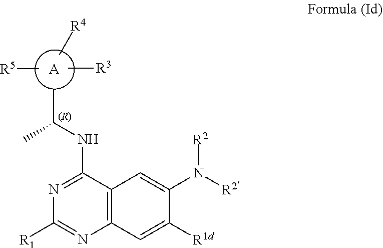
or a pharmaceutically acceptable salt or a pharmaceutically acceptable regioisomer thereof;
wherein A, the groups R¹, R², R^{2′}, R³, R⁴, R⁵ are as defined in claim 1.

8. (canceled)

9. The compound of formula (I) according to claim 1, are to novel compounds of formula (If) 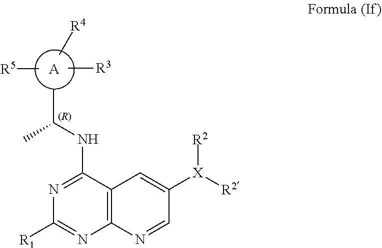
or a pharmaceutically acceptable salt or a pharmaceutically acceptable regioisomer thereof;
A, the groups R¹, R², R^{2′}, R³, R⁴, R⁵ are as defined in claim 1.

10. (canceled)

11. The compound of formula (I) according to claim 1, are to novel compounds of formula (Ih) 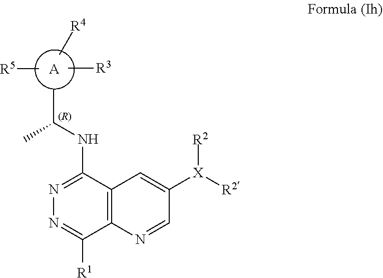
or a pharmaceutically acceptable salt or a pharmaceutically acceptable regioisomer thereof;
A, the groups R¹, R², R^{2′}, R³, R⁴, R⁵ are as defined in claim 1.

12. (canceled)

13. (canceled)

14. The compound of formula (I) according to claim 1, are to novel compounds of formula (Ik) 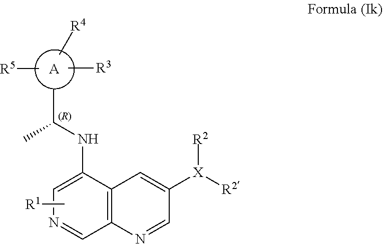
or a pharmaceutically acceptable salt or a pharmaceutically acceptable regioisomer thereof;
wherein A, the groups R¹, R², R^{2′}, R³, R⁴, R⁵ are as defined in claim 1.

15. The compound of formula (I) according to claim 1, are to novel compounds of formula (II) 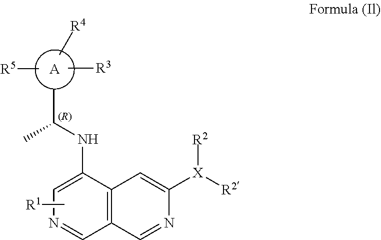
or a pharmaceutically acceptable salt or a pharmaceutically acceptable regioisomer thereof;
wherein A, the groups R¹, R², R^{2′}, R³, R⁴, R⁵ are as defined in claim 1.

16. The compound of formula (I) according to claim 1, are to novel compounds of formula (Im) 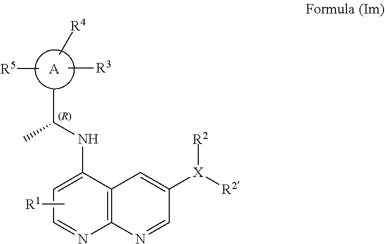
or a pharmaceutically acceptable salt or a pharmaceutically acceptable regioisomer thereof;
wherein A, the groups R¹, R², R^{2′},R³, R⁴, R⁵ are as defined in claim 1.

17. (canceled)

18. (canceled)

19. A compound is selected from the group consisting of:
(R)—N-(1-(3-amino-5-(trifluoromethyl)phenyl)ethyl)-2-chloro-6-(pyrrolidin-1-yl)pyrido[3,4-d]pyrimidin-4-amine
N4-(R)-(1-(3-amino-5-(trifluoromethyl)phenyl)ethyl)-N2,N2-dimethyl-6-(pyrrolidin-1-yl)pyrido[3,4-d]pyrimidine-2,4-diamine
(R)—N-(1-(3-amino-5-(trifluoromethyl)phenyl)ethyl)-2-methoxy-6-(pyrrolidin-1-yl)pyrido[3,4-d]pyrimidin-4-amine
(R)—N-(1-(3-amino-5-(trifluoromethyl)phenyl)ethyl)-2-(2-methoxyethoxy)-6-(pyrrolidin-1-yl)pyrido[3,4-d]pyrimidin-4-amine
(R)—N-(1-(3-amino-5-(trifluoromethyl)phenyl)ethyl)-2-morpholino-6-(pyrrolidin-1-yl)pyrido[3,4-d]pyrimidin-4-amine
(R)—N-(1-(3-amino-5-(trifluoromethyl)phenyl)ethyl)-2-(4-methylpiperazin-1-yl)-6-(pyrrolidin-1-yl)pyrido[3,4-d]pyrimidin-4-amine
(R)—N4-(1-(3-amino-5-(trifluoromethyl)phenyl)ethyl)-N2-methyl-6-(pyrrolidin-1-yl)pyrido[3,4-d]pyrimidine-2,4-diamine
(R)—N4-(1-(3-amino-5-(trifluoromethyl)phenyl)ethyl)-N2,N2-dimethyl-6-morpholinopyrido[3,4-d]pyrimidine-2,4-diamine
(R)—N4-(1-(3-amino-5-(trifluoromethyl)phenyl)ethyl)-N2-(2-(dimethylamino)ethyl)-6-(pyrrolidin-1-yl)pyrido[3,4-d]pyrimidine-2,4-diamine
(R)—N4-(1-(3-amino-5-(trifluoromethyl)phenyl)ethyl)-N2-(2-(dimethylamino)ethyl)-N2-methyl-6-(pyrrolidin-1-yl)pyrido[3,4-d]pyrimidine-2,4-diamine
(R)-2-((4-((1-(3-amino-5-(trifluoromethyl)phenyl)ethyl)amino)-6-(pyrrolidin-1-yl)pyrido[3,4-d]pyrimidin-2-yl)oxy)ethan-1-ol
(R)-2-((4-((1-(3-amino-5-(trifluoromethyl)phenyl)ethyl)amino)-6-(pyrrolidin-1-yl)pyrido[3,4-d]pyrimidin-2-yl)amino)ethan-1-ol
(R)-2-((4-((1-(3-amino-5-(trifluoromethyl)phenyl)ethyl)amino)-6-(pyrrolidin-1-yl)pyrido[3,4-d]pyrimidin-2-yl)(methyl)amino)ethan-1-ol
(R)—N4-(1-(3-amino-5-(trifluoromethyl)phenyl)ethyl)-N2-(2-methoxyethyl)-6-(pyrrolidin-1-yl)pyrido[3,4-d]pyrimidine-2,4-diamine
(R)—N4-(1-(3-amino-5-(trifluoromethyl)phenyl)ethyl)-N2-(2-methoxyethyl)-N2-methyl-6-(pyrrolidin-1-yl)pyrido[3,4-d]pyrimidine-2,4-diamine
(R)—N-(1-(3-amino-5-(trifluoromethyl)phenyl)ethyl)-2-(azetidin-1-yl)-6-(pyrrolidin-1-yl)pyrido[3,4-d]pyrimidin-4-amine
(R)—N-(1-(3-amino-5-(trifluoromethyl)phenyl)ethyl)-2,6-di(pyrrolidin-1-yl)pyrido[3,4-d]pyrimidin-4-amine
(R)—N4-(1-(3-amino-5-(trifluoromethyl)phenyl)ethyl)-N2-methyl-6-morpholinopyrido[3,4-d]pyrimidine-2,4-diamine
(R)—N-(1-(3-amino-5-(trifluoromethyl)phenyl)ethyl)-2-chloro-6-morpholinopyrido[3,4-d]pyrimidin-4-amine
(R)—N-(1-(3-amino-5-(trifluoromethyl)phenyl)ethyl)-2-chloro-6-(4-methoxypiperidin-1-yl)pyrido[3,4-d]pyrimidin-4-amine
(R)—N-(1-(3-amino-5-(trifluoromethyl)phenyl)ethyl)-2-chloro-6-(4,4-difluoropiperidin-1-yl)pyrido[3,4-d]pyrimidin-4-amine
(R)—N-(1-(3-amino-5-(trifluoromethyl)phenyl)ethyl)-2-chloro-6-(4-fluoropiperidin-1-yl)pyrido[3,4-d]pyrimidin-4-amine
(R)—N-(1-(3-amino-5-(trifluoromethyl)phenyl)ethyl)-2-chloro-6-(4-methylpiperazin-1-yl)pyrido[3,4-d]pyrimidin-4-amine
(R)—N-(1-(3-amino-5-(trifluoromethyl)phenyl)ethyl)-2-chloro-6-(4-ethylpiperazin-1-yl)pyrido[3,4-d]pyrimidin-4-amine
(R)—N-(1-(3-amino-5-(trifluoromethyl)phenyl)ethyl)-2-chloro-6-(4-isopropylpiperazin-1-yl)pyrido[3,4-d]pyrimidin-4-amine
(R)—N-(1-(3-amino-5-(trifluoromethyl)phenyl)ethyl)-2-chloro-6-(4-cyclopropylpiperazin-1-yl)pyrido[3,4-d]pyrimidin-4-amine
(R)—N-(1-(3-amino-5-(trifluoromethyl)phenyl)ethyl)-2-chloro-6-(4-methylpiperidin-1-yl)pyrido[3,4-d]pyrimidin-4-amine
(R)—N-(1-(3-amino-5-(trifluoromethyl)phenyl)ethyl)-2-chloro-6-(4-cyclobutylpiperazin-1-yl)pyrido[3,4-d]pyrimidin-4-amine
(R)-1-(4-((1-(3-amino-5-(trifluoromethyl)phenyl)ethyl)amino)-2-chloropyrido[3,4-d]pyrimidin-6-yl)piperidin-4-ol
(R)—N4-(1-(3-amino-5-(trifluoromethyl)phenyl)ethyl)-6-(4-methoxypiperidin-1-yl)-N2-methylpyrido[3,4-d]pyrimidine-2,4-diamine
(R)—N4-(1-(3-amino-5-(trifluoromethyl)phenyl)ethyl)-N2-methyl-6-(4-methylpiperazin-1-yl)pyrido[3,4-d]pyrimidine-2,4-diamine
(R)—N4-(1-(3-amino-5-(trifluoromethyl)phenyl)ethyl)-6-(4,4-difluoropiperidin-1-yl)-N2-methylpyrido[3,4-d]pyrimidine-2,4-diamine
(R)—N4-(1-(3-amino-5-(trifluoromethyl)phenyl)ethyl)-6-(4-fluoropiperidin-1-yl)-N2-methylpyrido[3,4-d]pyrimidine-2,4-diamine
(R)-1-(4-((1-(3-amino-5-(trifluoromethyl)phenyl)ethyl)amino)-6-(pyrrolidin-1-yl)pyrido[3,4-d]pyrimidin-2-yl)pyrrolidin-2-one
(R)—N4-(1-(3-amino-5-(trifluoromethyl)phenyl)ethyl)-N2-(2-(methylamino)ethyl)-6-morpholinopyrido[3,4-d]pyrimidine-2,4-diamine
(R)—N4-(1-(3-amino-5-(trifluoromethyl)phenyl)ethyl)-N2-(2-methoxyethyl)-6-morpholinopyrido[3,4-d]pyrimidine-2,4-diamine
(R)—N-(1-(3-amino-5-(trifluoromethyl)phenyl)ethyl)-2-(azetidin-1-yl)-6-morpholinopyrido[3,4-d]pyrimidin-4-amine
(R)—N4-(1-(3-amino-5-(trifluoromethyl)phenyl)ethyl)-N2-isopropyl-6-morpholinopyrido[3,4-d]pyrimidine-2,4-diamine
(R)—N4-(1-(3-amino-5-(trifluoromethyl)phenyl)ethyl)-N2-(2-(methylamino)ethyl)-6-(pyrrolidin-1-yl)pyrido[3,4-d]pyrimidine-2,4-diamine
(R)—N-(1-(3-amino-5-(trifluoromethyl)phenyl)ethyl)-2-(azetidin-1-yl)-6-(4-methoxypiperidin-1-yl)pyrido[3,4-d]pyrimidin-4-amine
(R)—N4-(1-(3-amino-5-(trifluoromethyl)phenyl)ethyl)-6-(4-methoxypiperidin-1-yl)-N2,N2-dimethylpyrido[3,4-d]pyrimidine-2,4-diamine
(R)—N4-(1-(3-amino-5-(trifluoromethyl)phenyl)ethyl)-N2-isopropyl-6-(4-methoxypiperidin-1-yl)pyrido[3,4-d]pyrimidine-2,4-diamine
(R)—N4-(1-(3-amino-5-(trifluoromethyl)phenyl)ethyl)-6-(4-fluoropiperidin-1-yl)-N2,N2-dimethylpyrido[3,4-d]pyrimidine-2,4-diamine
(R)—N-(1-(3-amino-5-(trifluoromethyl)phenyl)ethyl)-2-(azetidin-1-yl)-6-(4-fluoropiperidin-1-yl)pyrido[3,4-d]pyrimidin-4-amine
(R)—N4-(1-(3-amino-5-(trifluoromethyl)phenyl)ethyl)-N2,N2-dimethyl-6-(4-methylpiperazin-1-yl)pyrido[3,4-d]pyrimidine-2,4-diamine
(R)—N-(1-(3-amino-5-(trifluoromethyl)phenyl)ethyl)-2-(azetidin-1-yl)-6-(4-methylpiperazin-1-yl)pyrido[3,4-d]pyrimidin-4-amine
(R)—N-(1-(3-amino-5-(trifluoromethyl)phenyl)ethyl)-2-(azetidin-1-yl)-6-(4-methylpiperidin-1-yl)pyrido[3,4-d]pyrimidin-4-amine
(R)—N-(1-(3-amino-5-(trifluoromethyl)phenyl)ethyl)-2-(azetidin-1-yl)-6-(4-ethylpiperazin-1-yl)pyrido[3,4-d]pyrimidin-4-amine
(R)—N4-(1-(3-amino-5-(trifluoromethyl)phenyl)ethyl)-6-(4-ethylpiperazin-1-yl)-N2,N2-dimethylpyrido[3,4-d]pyrimidine-2,4-diamine
(R)—N4-(1-(3-amino-5-(trifluoromethyl)phenyl)ethyl)-6-(4-isopropylpiperazin-1-yl)-N2,N2-dimethylpyrido[3,4-d]pyrimidine-2,4-diamine
(R)—N-(1-(3-amino-5-(trifluoromethyl)phenyl)ethyl)-2-(azetidin-1-yl)-6-(4,4-difluoropiperidin-1-yl)pyrido[3,4-d]pyrimidin-4-amine
(R)—N-(1-(3-amino-5-(trifluoromethyl)phenyl)ethyl)-2-(azetidin-1-yl)-6-(4-cyclobutylpiperazin-1-yl)pyrido[3,4-d]pyrimidin-4-amine
(R)—N4-(1-(3-amino-5-(trifluoromethyl)phenyl)ethyl)-2-(azetidin-1-yl)-N6,N6-dimethylpyrido[3,4-d]pyrimidine-4,6-diamine
(R)—N4-(1-(3-amino-5-(trifluoromethyl)phenyl)ethyl)-2-(azetidin-1-yl)-N6-isopropylpyrido[3,4-d]pyrimidine-4,6-diamine
(R)—N4-(1-(3-amino-5-(trifluoromethyl)phenyl)ethyl)-N6-isopropyl-2-(pyrrolidin-1-yl)pyrido[3,4-d]pyrimidine-4,6-diamine
(R)—N4-(1-(3-amino-5-(trifluoromethyl)phenyl)ethyl)-2-(azetidin-1-yl)-N6-methylpyrido[3,4-d]pyrimidine-4,6-diamine
(R)—N-(1-(3-amino-5-(trifluoromethyl)phenyl)ethyl)-2-methoxy-6-morpholinopyrido[3,4-d]pyrimidin-4-amine
(R)—N-(1-(3-amino-5-(trifluoromethyl)phenyl)ethyl)-2-methoxy-6-(4-methoxypiperidin-1-yl)pyrido[3,4-d]pyrimidin-4-amine
(R)-2-chloro-N-(1-(3-(difluoromethyl)-2-fluorophenyl)ethyl)-6-morpholinopyrido[3,4-d]pyrimidin-4-amine
(R)-2-chloro-N-(1-(3-(difluoromethyl)-2-fluorophenyl)ethyl)-6-(4-methoxypiperidin-1-yl)pyrido[3,4-d]pyrimidin-4-amine
(R)-2-chloro-N-(1-(3-(difluoromethyl)-2-fluorophenyl)ethyl)-6-(4-methylpiperazin-1-yl)pyrido[3,4-d]pyrimidin-4-amine
(R)-2-chloro-N-(1-(3-(difluoromethyl)-2-fluorophenyl)ethyl)-6-(4-ethylpiperazin-1-yl)pyrido[3,4-d]pyrimidin-4-amine
(R)—N-(1-(3-amino-5-(trifluoromethyl)phenyl)ethyl)-2-chloro-6-(tetrahydro-2H-pyran-4-yl)pyrido[3,4-d]pyrimidin-4-amine
(R)-2-chloro-N-(1-(3-(difluoromethyl)-2-fluorophenyl)ethyl)-6-(tetrahydro-2H-pyran-4-yl)pyrido[3,4-d]pyrimidin-4-amine
(R)—N4-(1-(3-amino-5-(trifluoromethyl)phenyl)ethyl)-N2,N2-dimethyl-6-(tetrahydro-2H-pyran-4-yl)pyrido[3,4-d]pyrimidine-2,4-diamine
(R)—N4-(1-(3-amino-5-(trifluoromethyl)phenyl)ethyl)-N2-methyl-6-(tetrahydro-2H-pyran-4-yl)pyrido[3,4-d]pyrimidine-2,4-diamine
(R)—N-(1-(3-amino-5-(trifluoromethyl)phenyl)ethyl)-2-(azetidin-1-yl)-6-(tetrahydro-2H-pyran-4-yl)pyrido[3,4-d]pyrimidin-4-amine
(R)—N-(1-(3-amino-5-(trifluoromethyl)phenyl)ethyl)-2-(azetidin-1-yl)-6-(1-methylpiperidin-4-yl)pyrido[3,4-d]pyrimidin-4-amine
(R)—N4-(1-(3-amino-5-(trifluoromethyl)phenyl)ethyl)-N2,N2-dimethyl-6-(1-methylpiperidin-4-yl)pyrido[3,4-d]pyrimidine-2,4-diamine
(R)—N-(1-(3-amino-5-(trifluoromethyl)phenyl)ethyl)-2-chloro-6-(4-ethylpiperazin-1-yl)-7-fluoroquinazolin-4-amine
(R)—N-(1-(3-amino-5-(trifluoromethyl)phenyl)ethyl)-2-chloro-6-(4-ethylpiperazin-1-yl)-7-methylquinazolin-4-amine
(R)—N-(1-(3-amino-5-(trifluoromethyl)phenyl)ethyl)-2-chloro-6-(4-ethylpiperazin-1-yl)-7-methoxyquinazolin-4-amine
(R)—N-(1-(3-amino-5-(trifluoromethyl)phenyl)ethyl)-2-chloro-6-(4-ethylpiperazin-1-yl)quinazolin-4-amine
(R)—N4-(1-(3-amino-5-(trifluoromethyl)phenyl)ethyl)-N2-methyl-6-morpholinoquinazoline-2,4-diamine
(R)—N-(4-((1-(3-amino-5-(trifluoromethyl)phenyl)ethyl)amino)-6-(pyrrolidin-1-yl)pyrido[3,4-d]pyrimidin-2-yl)propionamide
(R)—N-(4-((1-(3-amino-5-(trifluoromethyl)phenyl)ethyl)amino)-6-(pyrrolidin-1-yl)pyrido[3,4-d]pyrimidin-2-yl)-2-methoxyacetamide
(R)-2-amino-N-(4-((1-(3-amino-5-(trifluoromethyl)phenyl)ethyl)amino)-6-(pyrrolidin-1-yl)pyrido[3,4-d]pyrimidin-2-yl)acetamide hydro chloride
(R)—N-(4-((1-(3-amino-5-(trifluoromethyl)phenyl)ethyl)amino)-6-(pyrrolidin-1-yl)pyrido[3,4-d]pyrimidin-2-yl)-3-(methylamino)propenamide
(R)—N-(4-((1-(3-amino-5-(trifluoromethyl)phenyl)ethyl)amino)-6-morpholinopyrido[3,4-d]pyrimidin-2-yl)-3-(methylamino)propanamide
(R)—N-(1-(3-amino-5-(trifluoromethyl)phenyl)ethyl)-2-chloro-6-(4-ethylpiperazin-1-yl)pyrido[2,3-d]pyrimidin-4-amine
(R)—N-(1-(3-amino-5-(trifluoromethyl)phenyl)ethyl)-6-(4-ethylpiperazin-1-yl)-2-methoxypyrido[2,3-d]pyrimidin-4-amine
(R)—N-(1-(3-amino-5-(trifluoromethyl)phenyl)ethyl)-2-chloro-6-(4-methylpiperazin-1-yl)pyrido[2,3-d]pyrimidin-4-amine
(R)—N-(1-(3-amino-5-(trifluoromethyl)phenyl)ethyl)-2-chloro-6-(piperidin-1-yl)pyrido[2,3-d]pyrimidin-4-amine
(R)—N-(1-(3-amino-5-(trifluoromethyl)phenyl)ethyl)-2-chloro-6-morpholinopyrido[2,3-d]pyrimidin-4-amine
(R)—N-(1-(3-amino-5-(trifluoromethyl)phenyl)ethyl)-6-(4-ethylpiperazin-1-yl)pyrido[3,4-c]pyridazin-4-amine
(R)—N-(1-(3-amino-5-(trifluoromethyl)phenyl)ethyl)-3-(4-ethylpiperazin-1-yl)-8-methylpyrido[2,3-d]pyridazin-5-amine
(R)—N-(1-(3-amino-5-(trifluoromethyl)phenyl)ethyl)-8-methyl-3-morpholinopyrido[2,3-d]pyridazin-5-amine
(R)—N-(1-(3-amino-5-(trifluoromethyl)phenyl)ethyl)-7-chloro-3-(4-ethylpiperazin-1-yl)-1,6-naphthyridin-5-amine
(R)—N-(1-(3-amino-5-(trifluoromethyl)phenyl)ethyl)-7-chloro-3-morpholino-1,6-naphthyridin-5-amine
N4-((R)-1-(3-amino-5-(trifluoromethyl)phenyl)ethyl)-N2-methylsulfonyl(pyrrolidin-1-yl)pyrido[3,4-d]pyrimidine-2,4-diamine
N4-((R)-1-(3-amino-5-(trifluoromethyl)phenyl)ethyl)-N2-aminosulfonyl(pyrrolidin-1-yl)pyrido[3,4-d]pyrimidine-2,4-diamine
N4-((R)-1-(3-amino-5-(trifluoromethyl)phenyl)ethyl)-N6-methyl-2-(pyrrolidin-1-yl)pyrido[3,4-d]pyrimidine-4,6-diamine
N4-((R)-1-(3-amino-5-(trifluoromethyl)phenyl)ethyl)-6-cyclopropyl-N2-methylpyrido[3,4-d]pyrimidine-2,4-diamine
N4-((R)-1-(3-amino-5-(trifluoromethyl)phenyl)ethyl)-6-cyclobutyl-N2-methylpyrido[3,4-d]pyrimidine-2,4-diamine
N-(4-((R)-1-(3-amino-5-(trifluoromethyl)phenyl)ethylamino)-6-(pyrrolidin-1-yl)pyrido[3,4-d]pyrimidin-2-yl)-2-aminoacetamide
N-(4-((R)-1-(3-amino-5-(trifluoromethyl)phenyl)ethylamino)-6-(pyrrolidin-1-yl)pyrido[3,4-d]pyrimidin-2-yl)-2-(methylamino) 2-aminoacetamide
N-(4-((R)-1-(3-amino-5-(trifluoromethyl)phenyl)ethylamino)-6-(pyrrolidin-1-yl)pyrido[3,4-d]pyrimidin-2-yl)-3-(methylamino)propanamide
4-(4-((R)-1-(3-amino-5-(trifluoromethyl)phenyl)ethylamino)-2-(methylamino)pyrido[3,4-d]pyrimidin-6-yl)morpholin-3-one
1-(4-((R)-1-(3-amino-5-(trifluoromethyl)phenyl)ethylamino)-2-(methylamino)pyrido[3,4-d]pyrimidin-6-yl)pyrrolidin-2-one
4-(4-((R)-1-(3-amino-5-(trifluoromethyl)phenyl)ethylamino)-2-(methylamino)pyrido[3,4-d]pyrimidin-6-yl)morpholin-2-one
1-(4-((R)-1-(3-amino-5-(trifluoromethyl)phenyl)ethylamino)-2-(methylamino)pyrido[3,4-d]pyrimidin-6-yl)piperidin-4-ol
N4-((R)-1-(4-(trifluoromethyl)thiophen-2-yl)ethyl)-N2-methyl-6-morpholinopyrido[3,4-d]pyrimidine-2,4-diamine
N4-((R)-1-(3-fluoro-4-(difluoromethyl)thiophen-2-yl)ethyl)-N2-methyl-6-morpholinopyrido[3,4-d]pyrimidine-2,4-diamine
1-(4-((R)-1-(3-amino-5-(trifluoromethyl)phenyl)ethylamino)-6-(methylamino)pyrido[3,4-d]pyrimidin-2-yl)pyrrolidin-2-one
N—((R)-1-(3-amino-5-(trifluoromethyl)phenyl)ethyl)-6-methoxy-2-(pyrrolidin-1-yl)pyrido[3,4-d]pyrimidin-4-amine
(R)-1-(4-(4-((1-(3-amino-5-(trifluoromethyl)phenyl)ethyl)amino)-2-chloropyrido[2,3-d]pyrimidin-6-yl)piperazin-1-yl)ethan-1-one
(R)—N-(1-(3-amino-5-(trifluoromethyl)phenyl)ethyl)-2-chloro-6-(4-ethylpiperazin-1-yl)-7-methylpyrido[2,3-d]pyrimidin-4-amine
(R)-2-(4-(4-((1-(3-amino-5-(trifluoromethyl)phenyl)ethyl)amino)-2-chloropyrido[2,3-d]pyrimidin-6-yl)piperazin-1-yl)ethan-1-ol
(R)—N-(1-(3-amino-5-(trifluoromethyl)phenyl)ethyl)-2-chloro-6-(4-(2-methoxyethyl)piperazin-1-yl)pyrido[2,3-d]pyrimidin-4-amine
(R)—N-(1-(3-amino-5-(trifluoromethyl)phenyl)ethyl)-2-chloro-6-(tetrahydro-2H-pyran-4-yl)pyrido[2,3-d]pyrimidin-4-amine
(R)—N-(1-(3-amino-5-(trifluoromethyl)phenyl)ethyl)-3-(4-ethylpiperazin-1-yl)-8-fluoropyrido[2,3-d]pyridazin-5-amine
(R)-4-((1-(3-amino-5-(trifluoromethyl)phenyl)ethyl)amino)-2-chloro-6-(4-ethylpiperazin-1-yl)-7-methylpyrido[3,4-d]pyrimidin-8(7H)-one
(R)-4-((1-(3-amino-5-(trifluoromethyl)phenyl)ethyl)amino)-6-(4-ethylpiperazin-1-yl)-2,7-dimethylpyrido[3,4-d]pyrimidin-8(7H)-one
(R)—N-(1-(3-amino-5-(trifluoromethyl)phenyl)ethyl)-3-(4-ethylpiperazin-1-yl)-1,7-naphthyridin-5-amine
(R)—N-(1-(3-amino-5-(trifluoromethyl)phenyl)ethyl)-6-(4-ethylpiperazin-1-yl)-2,7-naphthyridin-4-amine
(R)—N-(1-(3-amino-5-(trifluoromethyl)phenyl)ethyl)-2-chloro-6-morpholino-1,8-naphthyridin-4-amine
(R)—N-(1-(3-amino-5-(trifluoromethyl)phenyl)ethyl)-2-methyl-6-morpholino-1,8-naphthyridin-4-amine
(R)—N-(1-(3-amino-5-(trifluoromethyl)phenyl)ethyl)-3-(4-ethylpiperazin-1-yl)-8-methyl-1,7-naphthyridin-5-amine
(R)—N-(1-(3-amino-5-(trifluoromethyl)phenyl)ethyl)-3-(4-ethylpiperazin-1-yl)-8-methyl-1,6-naphthyridin-5-amine
(R)-1-((1-(3-amino-5-(trifluoromethyl)phenyl)ethyl)amino)-7-(4-ethylpiperazin-1-yl)-6-methylpyrido[3,4-d]pyridazin-5(6H)-one
(R)-1-((1-(3-amino-5-(trifluoromethyl)phenyl)ethyl)amino)-7-(4-ethylpiperazin-1-yl)-4,6-dimethylpyrido[3,4-d]pyridazin-5(6H)-one
(R)-1-((1-(3-amino-5-(trifluoromethyl)phenyl)ethyl)amino)-7-(4-ethylpiperazin-1-yl)-4-fluoro-6-methylpyrido[3,4-d]pyridazin-5(6H)-one
(R)—N-(1-(3-amino-5-(trifluoromethyl)phenyl)ethyl)-2-chloro-6-(1-ethylpiperidin-4-yl)-7-methyl-6,7-dihydropyrimido[4,5-d]pyridazin-4-amine or a pharmaceutically acceptable salt thereof or a pharmaceutically acceptable regioisomer thereof.

20. The pharmaceutical composition, comprising at least one compound according to claim 1, or a pharmaceutically acceptable salt or a stereoisomer thereof and a pharmaceutically acceptable carrier or excipient.

21. The compound according to claim 1, or a pharmaceutically acceptable salt or a stereoisomer thereof, for use as a medicament.

22. The method of inhibiting SOS1:KRAS protein-protein interaction comprising administering a therapeutically effective amount of a compound according to claim 1.

23. The method as claimed in claim 22 for the treatment diseases or disorder associated with SOS1KRAS protein-protein interaction.

24. The pharmaceutical composition, comprising at least one compound according to claim 19, or a pharmaceutically acceptable salt or a stereoisomer thereof and a pharmaceutically acceptable carrier or excipient.

25. The compound according to claim 19, or a pharmaceutically acceptable salt or a stereoisomer thereof, for use as a medicament.

26. The method of inhibiting SOS1:KRAS protein-protein interaction comprising administering a therapeutically effective amount of a compound according to claim 19.

27. The method as claimed in claim 26 for the treatment diseases or disorder associated with SOS1KRAS protein-protein interaction.
